(19) United States
(12) Patent Application Publication
(10) Pub. No.: US 2026/0248899 A1
(43) Pub. Date: Aug. 27, 2026

(54) PEPTIDE LINKERS FOR MODULATING ANTIGEN IMMUNE RESPONSE IN CANCER VACCINES

(71) Applicant: NORTHWESTERN UNIVERSITY, Evanston, IL (US)

(72) Inventors: Chad A. Mirkin, Wilmette, IL (US); Tanner D. Fink, Evanston, IL (US)

(21) Appl. No.: 19/545,780

(22) Filed: Feb. 20, 2026

Related U.S. Application Data
(60) Provisional application No. 63/761,733, filed on Feb. 21, 2025.

(51) Int. Cl. A61K 39/00; A61K 39/385; A61K 47/62; A61K 47/69; A61P 35/00

(52) U.S. Cl. CPC A61K 39/001192; A61K 39/001111; A61K 39/385; A61K 47/62; A61K 47/6929; A61P 35/00; A61K 2039/55555; A61K 2039/55561; A61K 2039/572; A61K 2039/6025; A61K 2039/627

(57) ABSTRACT

The disclosure generally relates to Cathepsin S-sensitive linkers, short amino acid sequences attached to antigens that form a structure termed a hybrid peptide. Further disclosed are spherical nucleic acids (SNAs), nanostructures with a core surrounded by a shell of oligonucleotides, wherein in some embodiments the hybrid peptide is attached to one or more oligonucleotides in the shell of oligonucleotides. In some aspects, the disclosure provides a SNA comprising: (a) a nanoparticle core; (b) a shell of oligonucleotides comprising one or more oligonucleotides attached to the nanoparticle core; and (c) a hybrid peptide attached to one or more oligonucleotides in the shell of oligonucleotides, wherein the hybrid peptide comprises a Cathepsin S-sensitive linker and an antigen.

### CROSS-REFERENCE TO RELATED APPLICATIONS

This application claims the priority benefit under 35 U.S.C. § 119(e) of U.S. Provisional Patent Application No. 63/761,733, filed Feb. 21, 2025, which is incorporated herein by reference in its entirety.

### INCORPORATION BY REFERENCE OF MATERIAL SUBMITTED ELECTRONICALLY

The Sequence Listing, which is a part of the present disclosure, is submitted concurrently with the specification as a text file. The name of the text file containing the Sequence Listing is “2024-124_SeqListing.xml”, which was created on Feb. 20, 2026 and is 16,735 bytes in size. The subject matter of the Sequence Listing is incorporated herein in its entirety by reference.

### BACKGROUND

To successfully elicit robust immune responses, it is necessary to consider the state in which vaccine components are presented to an immune cell and how they are processed intracellularly.

### SUMMARY

To mount a robust and durable immune response, the antigen in a vaccine must be processed efficiently by antigen-presenting immune cells. The present disclosure provides an approach for optimizing antigen processing by immune cells and the efficacy of immune system induction by using the modular design of spherical nucleic acid (SNA) nanostructures, and a hybrid peptide that releases antigens in their native state at specific locations within the cell. The SNAs and hybrid peptides disclosed herein take advantage of the cleavage selectivity and specificity of Cathepsin S, a cysteine protease known to process endocytosed antigens for cross-presentation on MHC class I complexes. In some aspects, the disclosure provides a hybrid peptide comprising an antigen and a Cathepsin S-sensitive linker, wherein the Cathepsin S-sensitive linker attaches the antigen to the SNA and releases the antigen in its native state in the cellular compartments containing Cathepsin S, which include the endosomes. Without wishing to be bound to any particular theory, one advantage of the disclosure is that the Cathepsin-S sensitive linker attached to the antigen can predictably release the antigen in its native state upon exposure to Cathepsin S, leading to preferential loading into MHC class I and a subsequent CD8⁺ T cell response mounted against the antigen.

The controlled intracellular delivery of therapeutic molecules in a desirable and methodological manner to elicit a robust therapeutic response is a challenging but attractive goal, particularly for immunotherapies. Current intracellular delivery platforms often rely on degradation or small-molecule sensitive chemically labile linkers for the intracellular delivery of therapeutic cargo. However, these approaches often provide little control over the spatial localization of payload delivery, and additionally, their intracellular release kinetics are difficult to predict and systemically control.

Disclosed herein are peptide-based protease sensitive linkers which can efficiently deliver peptide antigens in their native and most immunogenic state into endosomes, where the antigen can then be processed to result in a robust immune response. This is achieved through the proteolysis of the linker by proteolytic endosomal enzymes, for example Cathepsin S, which is a protease known to play key roles in antigen degradation and is specifically enriched in antigen presenting cells. These non-biologically derived linkers were designed based on the cleavage selectivity and specificity of Cathepsin S, and can be synthesized onto the N-terminus of peptide-based antigens directly during solid-state peptide synthesis. Upon proteolysis by Cathepsin S, the linkers are cleaved at a specific site which releases the antigen in its unmodified and immunogenic state.

The linkers disclosed herein are applicable to antigen sequences with different primary sequences, hydrophobicity, and charge, demonstrating their versatility. Importantly, site specific modifications in the linker sequence provide temporal control over cleavage kinetics and resulting release profiles. The linker-antigen hybrid peptides can be chemically conjugated to nanoparticle systems, including the spherical nucleic acid (SNA) nanoarchitecture, in some embodiments utilizing covalent, non-cleavable chemistries while still maintaining their protease-sensitive nature. These protease-sensitive SNA nanoconstructs result in heightened immune activation both in vitro and in vivo compared to compositionally identical SNA counterparts containing small-molecule chemically labile linkers, thus demonstrating their superiority over conventional approaches.

Applications for the technology disclosed herein include, but are not limited to, anti-cancer vaccination, where the cancer includes, but is not limited to, breast cancer, colon cancer, liver cancer, pancreatic cancer, melanoma, ovarian cancer, and prostate cancer.

Advantages of the technology disclosed herein include, but are not limited to:
Site-specific cleavage by Cathepsin S and release of native antigen
Greater potency of the SNAs comprising the hybrid peptide compared to compositionally identical but chemically unique SNA designs (e.g., SDEC linker)
Robust induction of an immune response and subsequent tumor suppression by SNAs comprising the hybrid peptide (compared to SDEC and BMPS linkers)

Accordingly, in some aspects the disclosure provides a spherical nucleic acid (SNA) comprising: a) a nanoparticle core; b) a shell of oligonucleotides comprising one or more oligonucleotides attached to the nanoparticle core; and c) a hybrid peptide attached to one or more oligonucleotides in the shell of oligonucleotides, wherein the hybrid peptide comprises a Cathepsin S-sensitive linker and an antigen. In some embodiments, the Cathepsin S-sensitive linker is cleavable and traceless. In further embodiments, the hybrid peptide is cleaved between a P1 and a P1′ position. In some embodiments, the antigen comprises 8-11 amino acids. In further embodiments, the Cathepsin S-sensitive linker comprises a four amino acid sequence. In some embodiments, the Cathepsin S-sensitive linker comprises Gln-X-Met-Glu, wherein X is an amino acid. In some embodiments, the Cathepsin S-sensitive linker X is an amino acid selected from the group comprising Pro, Ser, and lie. In some embodiments, the Cathepsin S-sensitive linker further comprises a Gly amino acid residue at the N-terminus of the Cathepsin S-sensitive linker. In various embodiments, the Cathepsin S-sensitive linker has a cleavage probability value of at least 0.3, at least 0.4, at least 0.5, or at least 0.6. In some embodiments, the Cathepsin S-sensitive linker is attached to the N-terminus of the antigen. In some embodiments, the C-terminus of the Cathepsin S-sensitive linker is attached to the antigen. In various embodiments, one or more oligonucleotides in the shell of oligonucleotides comprises a double-stranded oligonucleotide. In some embodiments, each oligonucleotide in the shell of oligonucleotides is a double stranded oligonucleotide. In various embodiments, the shell of oligonucleotides comprises DNA, RNA, or a combination thereof. In various embodiments, the shell of oligonucleotides comprises a single-stranded DNA, a double-stranded DNA, a single-stranded RNA, a double-stranded RNA, or a combination thereof. In some embodiments, the hybrid peptide is attached to the double-stranded oligonucleotide. In further embodiments, the shell of oligonucleotides comprises an immunostimulatory oligonucleotide. In some embodiments, each oligonucleotide in the shell of oligonucleotides is an immunostimulatory oligonucleotide. In further embodiments, the immunostimulatory oligonucleotide is a CpG-motif containing oligonucleotide. In some embodiments, the immunostimulatory oligonucleotide is a toll-like receptor (TLR) agonist. In some embodiments, one strand of the double-stranded oligonucleotide in the shell of oligonucleotides comprises the nucleotide sequence of SEQ ID NO: 1, SEQ ID NO: 2, and/or SEQ ID NO: 3. In some embodiments, one strand of the double-stranded oligonucleotide in the shell of oligonucleotides comprises the nucleotide sequence of SEQ ID NO: 4, SEQ ID NO: 5, and/or SEQ ID NO: 6. In some embodiments, the hybrid peptide is attached to the 3′ end of an oligonucleotide in the shell of oligonucleotides. In some embodiments, the hybrid peptide is attached to the oligonucleotide via an additional linker. In various embodiments, the additional linker is a non-cleavable linker, a cleavable but not traceless linker, a cleavable and traceless linker, a disulfide linker, or a peptide-based linker. In some embodiments, the non-cleavable linker is N-β-maleimidopropyl-oxysuccinimide ester (BMPS). In various embodiments, the nanoparticle core is a liposomal core, a lipid nanoparticle core, a polymer core, a protein core, or an inorganic core. In some embodiments, one or more oligonucleotides in the shell of oligonucleotides is attached to the nanoparticle core via a lipid anchor group. In some embodiments, each oligonucleotide in the shell of oligonucleotides is attached to the nanoparticle core via a lipid anchor group. In various embodiments, the lipid anchor group comprises tocopherol, palmitoyl, dipalmitoyl, stearyl, distearyl, cholesterol, or dodecane anchor (C12)₉. In some embodiments, the antigen is a peptide. In various embodiments, the antigen is a tumor-associated antigen, a tumor specific antigen, a neoantigen, or a combination thereof. In various embodiments, the antigen is or comprises ovalbumin, OVA1, MSLN, p53, Ras, a melanoma related antigen, a HPV related antigen, a prostate cancer related antigen, an ovarian cancer related antigen, a breast cancer related antigen, a hepatocellular carcinoma related antigen, a colon cancer related antigen, a lung cancer related antigen, an osteocarcinoma related antigen, human papillomavirus (HPV) E6/E7 nuclear protein, or a combination thereof. In various embodiments, the antigen is SIINFEKL, KVPRNQDWL, TAPDNLGYA, or LCPGNKYEM.

In some aspects, the disclosure provides a hybrid peptide comprising a Cathepsin S-sensitive linker and an antigen, wherein the Cathepsin S-sensitive linker comprises Gln-X-Met-Glu, and wherein X is an amino acid. In some embodiments, the Cathepsin S-sensitive linker is cleavable and traceless. In some embodiments, the hybrid peptide is cleaved between a P1 and a P1′ position. In further embodiments, the antigen comprises 8-11 amino acids. In some embodiments, the Cathepsin S-sensitive linker comprises a four amino acid sequence. In some embodiments, the Cathepsin S-sensitive linker X is a hydrophobic amino acid. In some embodiments, the Cathepsin S-sensitive linker X is an amino acid selected from the group comprising Pro, Ser, and lie. In some embodiments, the Cathepsin S-sensitive linker further comprises a Gly amino acid residue at the N-terminus of the linker. In various embodiments, the Cathepsin S-sensitive linker has a cleavage probability value of at least 0.3, at least 0.4, at least 0.5, or at least 0.6. In some embodiments, the Cathepsin S-sensitive linker is attached to the N-terminus of the antigen. In some embodiments, the C-terminus of the Cathepsin S-sensitive linker is attached to the antigen.

The disclosure also provides, in various aspects, a composition comprising a plurality of the SNAs of the disclosure. In further aspects, the disclosure provides a composition comprising a plurality of the hybrid peptides of the disclosure. In some embodiments, at least two of the plurality comprise a different antigen relative to each other.

In further aspects, the disclosure provides a pharmaceutical formulation comprising an SNA, hybrid peptide, or composition of the disclosure and a pharmaceutically acceptable carrier, diluent, stabilizer, preservative, or adjuvant.

In still further aspects, the disclosure provides an antigenic composition comprising an SNA, hybrid peptide, composition, or pharmaceutical formulation of the disclosure, wherein the antigenic composition is capable of generating an immune response in a mammalian subject. In some embodiments, the immune response is a CD8⁺ T cell-mediated response.

In some aspects, the disclosure provides a method of producing an immune response in a subject, comprising administering to the subject an effective amount of the antigenic composition of the disclosure, thereby producing an immune response in the subject. In some embodiments, the immune response is a CD8⁺ T cell-mediated response. In some embodiments, the immune response is a CD44⁺/CD62L⁻ CD8⁺ effector memory T cell-mediated response. In some embodiments, the immune response is a KLRG1⁺/CD127⁻ short-lived effector T cell-mediated response.

### BRIEF DESCRIPTION OF THE DRAWINGS

FIG. 1 shows novel hybrid linker-antigen peptide design. FIG. 1A provides a schematic representation of peptide linker design utilizing model antigen SIINFEKL from ovalbumin system. Through meticulous arrangement of amino acids on the N-terminus of peptide antigens, linkers are designed to be cleaved between the C-terminus of the linker, and N-terminus of peptide antigen to release antigen in its native and most immunogenic state post proteolysis by Cathepsin S. FIG. 1B provides screening analysis of human Cathepsin S “cleavage probability”, Cathepsin S selectivity over Cathepsin B and Cathepsin L, and gravy score for the 160,000 possible peptide linkers for the P4-P1 linker sequence utilizing PICS database. FIG. 1C shows the three hybrid linker-antigen peptides chosen after initial screening to investigate cleavage kinetics post Cathepsin S proteolysis with their respective predicted properties. The alteration of amino acid identity in the P3 position to control overall peptide hydrophobicity was chosen to regulate cleavage kinetics. FIG. 1D provides chimera models of QPME-SIIN, QSME-SIIN, and QIME-SIIN docking to binding site of human Cathepsin S (1NPZ) for binding clusters which allow the cleavage site between E-S to be within 5 Å of active surface cysteine. Hydrophobicity of amino acid in P3 position results in different predicted binding affinities and conformations to the active site of Cathepsin S to control cleavage rates.

FIG. 2 shows the cleavage of hybrid linker-antigen peptides by human Cathepsin S and resulting kinetic fits. FIG. 2A provides representative TIC profile of GQPMESIINFEKL sample after incubation with Cathepsin S. A single product peak is observed, suggesting that proteolysis results in the formation of a single cleavage product.

FIG. 2B shows representative mass spectrum corresponding to product peak in TIC suggesting that single cleavage product after proteolysis is SIINFEKL antigen peptide without any additional modification, suggesting cleavage occurs between E-S of hybrid linker-antigen peptide. FIG. 2C provides Michaelis-Menten cleavage kinetics of Cathepsin S with three hybrid linker-antigen peptide substrates suggesting that peptide hydrophobicity can be used to regulate temporal release of antigen from hybrid linker-antigen peptides as the most hydrophilic peptide (GQPMESIINFEKL) is able to be cleaved the most efficiently, followed by the peptide with intermediate hydrophobicity (GQSMESIINFEKL), and the most hydrophobic (GQIMESIINFEKL). Data is presented as mean±STDEV, n=3.

FIG. 3 provides cleavage site analysis and degradation kinetics of hybrid peptide linkers cleaved by human and murine Cathepsin S. FIG. 3A shows a representative TIC profile of GQPMESIINFEKL sample after incubation with either mouse Cathepsin S (CaSM) or Human Cathepsin S (CaSH), showing the production of a single product (SIINFKEL) from proteolysis. FIG. 3B provides Michaelis-Menten kinetics of GQPMESIINFEKL degradation with mouse Cathepsin S (CaSM) and human Cathepsin S (CaSH) along with resulting fit parameters. High homology (>75%) and similarities in Michaelis-Menten kinetics suggest the hybrid linker-antigen peptides are applicable across mouse models and relevant to human systems.

FIG. 4 demonstrates the applicability of GQPME linker to other literature established MHC class I epitopes. TIC and mass spectra corresponding to degradation product peak for (FIG. 4A) GQPMEKVPRNQDWL (FIG. 4B) GQPMETAPDNLGYA, and (FIG. 4C) GQPMELCPGNKYEM incubated with Cathepsin S for 24 hrs, showing proteolysis results in the generation of the antigen peptide in its native state. FIG. 4D provides a table showing sequences, gravy scores, hydrophobicity, theoretical isoelectric point (IEP), and molecular weight of established MHC class I epitopes which can be released from the hybrid linker-antigen peptides, showing peptide linkers are applicable to antigen epitopes with different primary sequences, hydrophobicity, and charge.

FIG. 5 shows SNA design containing hybrid linker-antigen peptides and their characterization. FIG. 5A provides an illustration of DNA-peptide conjugates containing hybrid linker-OVA₂₅₇₋₂₆₄ peptides where peptides are conjugated to complementary DNA via a non-cleavable BMPS moiety. FIG. 5B provides an illustration of DNA-peptide conjugate containing OVA₂₅₇₋₂₆₄ peptide where peptide is conjugated to complementary DNA via traceless redox-responsive SDEC linker. FIG. 5C provides an illustration of DNA-peptide conjugate containing OVA₂₅₇₋₂₆₄ peptide where peptide is conjugated to complementary DNA via non-cleavable BMPS moiety. FIG. 5D provides an illustration of hybridized liposomal SNA nanoconstruct where oligonucleotide shell is comprised of immunostimulatory (CpG 1826) oligonucleotides hybridized with antigen conjugated complementary strand. The SNA architecture is formed from intercalation of cholesterol anchor on CpG strand into liposomal bilayer. FIG. 5E shows an agarose gel of fully synthesized SNAs containing non-cleavable (BMPS), redox-responsive (SDEC), and Cathepsin S cleavable (GQP, GQS, and GQI) linkers. All SNA groups independent of linker identity exhibit similar changes in electrophoretic mobility to free CpG DNA, indicative of SNA formation. FIG. 5F provides a characterization of bare liposomes and SNAs by number weighted DLS, n=4. All SNAs exhibit similar changes in apparent hydrodynamic diameter. FIG. 5G shows ζ-potential measurements of bare liposomes and SNAs showing change in surface properties due to arrangement of oligonucleotides on liposome surface from SNA formation, n=2. Data is represented as average±STDEV.

FIG. 6 shows the in vitro processing of SNA nanoconstructs formulated with compositionally identical but chemically unique designs. FIG. 6A provides the percent and MFI of bone-marrow-derived CD11c⁺ DC kinetics of CD80 expression over 48 hrs after pulsing with SNAs. FIG. 6B provides the percent and MFI of bone-marrow-derived CD11c⁺ DC kinetics of CD86 expression over 48 hrs of DCs pulsed with SNAs. Similarities between CD80 and CD86 over 48 hr time course suggests that SNAs with Cathepsin S peptide linkers do not inhibit TLR9 activation and downstream expression of costimulatory markers. FIG. 6C provides the percent and MFI of bone-marrow-derived CD11c⁺ DC kinetics of OVA₂₅₇₋₂₆₄ expression on MHC class I over 48 hrs of DCs pulsed with SNAs. Differences in kinetic profiles of SDEC-OVA₂₅₇₋₂₆₄/BMPS− OVA₂₅₇₋₂₆₄ SNAs and Cathepsin S hybrid linker-OVA₂₅₇₋₂₆₄ SNAs suggest differences in intracellular processing by utilizing different antigen presentation pathways. Data is presented as mean±STDEV, n=3.

FIG. 7 demonstrates an in vitro immune response to SNA treatment with OVA₂₅₇₋₂₆₄ antigen of OT-1 mouse splenocytes. FIG. 7A provides representative dose-responsive curves of T-cell proliferation generated from treating splenocytes using a range of SNA treatments. Duplicates were performed at each concentration. FIG. 7B provides calculated EC₅₀ values for each SNA treatment group from 3 independent T-cell proliferation assays. SNAs formulated with the GQI-OVA₂₅₇₋₂₆₄ hybrid peptide result in a 10-fold enhancement in EC₅₀, suggesting a more immunogenic construct over SDEC-OVA₂₅₇₋₂₆₄ SNA. Differences in EC₅₀ between the hybrid linker-OVA₂₅₇₋₂₆₄ peptides suggest the importance of cleavage kinetics in resulting immune response, and that immunogenicity is negatively correlated with cleavage rate. FIG. 7C provides exemplary CD8⁺ T cell proliferation histograms for GQI-OVA₂₅₇₋₂₆₄ and SDEC-OVA₂₅₇₋₂₆₄ SNAs treated at 100 pM by CpG DNA and peptide. FIG. 7D exhibits the relationship between −log(EC₅₀) and proteolytic cleavage of hybrid linker-OVA₂₅₇₋₂₆₄ peptides using Vₘₐₓ from Michaelis Menten kinetic fits showing negative correlation between potency and maximum proteolysis rate. Data is presented as mean±STDEV, n=3.

FIG. 8 shows that SNAs containing hybrid linker-antigen peptides elicit potent antigen-specific CD8+ T cell immunity to similar levels as redox-responsive SNAs over a long vaccination schedule. FIG. 8A provides the injection timeline for immunizations. FIG. 8B shows interferon gamma (INF-γ) spot forming cells (SFCs) after stimulation of splenocytes with SIINFEKL peptide antigen, n=4. FIG. 8C provides representative counted ELISpot images for SNA treatment groups. The treatment groups are, from top to bottom, BMPS-OVA₂₅₇₋₂₆₄, SDEC-OVA₂₅₇₋₂₆₄, GQP-OVA₂₅₇₋₂₆₄, GQI-OVA₂₅₇₋₂₆₄, and Naïve. FIG. 8D provides an assessment of polyfunctional CD8⁺ T cells expressing intracellular INF-γ and CD107a degranulation marker from splenocytes, n=3. FIG. 8E provides an assessment of effector memory phenotypes (CD44⁺/CD62L⁻) of CD8⁺ T cells from splenocytes, n=3. FIG. 8F exhibits the ability of isolated CD8⁺ T cells to induce killing of ovalbumin-expressing target cancer cells (E.G7-OVA) measured though cells double-positive for apoptotic (annexin V) and necrotic (7-AAD) markers, n=4. Significance between groups was determined by one-way ANOVA with Dunnett's multiple comparison test. *P<0.05, **P<0.01,****P<0.0001. Data presented as average±STDEV.

FIG. 9 demonstrates that hybrid linker-antigen peptide-based SNA cancer vaccines show enhanced antitumor effect in vivo compared to redox-responsive SNA design. FIG. 9A shows the dosing schedule for treatment of E.G7-OVA lymphoma tumors. FIG. 9B demonstrates initial E.G7-OVA tumor growth with different treatments including saline (PBS), SDEC-OVA₂₅₇₋₂₆₄ SNA, GQP-OVA₂₅₇₋₂₆₄ SNA, and GQI-OVA₂₅₇₋₂₆₄ SNA. FIG. 9C shows quantitative comparison of tumor volumes among treatment groups at day 21 post-tumor inoculation. FIG. 9D provides Kaplan-Meier survival curves for each treatment condition out to 42 days post tumor inoculation. Significance between groups was determined by one-way ANOVA with Dunnett's multiple comparison test. *P<0.05, ***P<0.001, ****P<0.0001. Data represented as average±SEM with n=9-10.

FIG. 10 depicts functional assessment of CD8⁺ OT-1 T cells following co-culture with bone marrow-derived CD11c⁺ dendritic cells (BMDCs) pre-treated with SNA formulations. FIG. 10A shows proliferation of CD8⁺ T cells following stimulation with SNAs comprising non-cleavable (BMPS), redox-responsive (SDEC), or Cathepsin S-cleavable hybrid linker-antigen peptides (n=3). SNAs comprising cleavable chemistries exhibit increased T cell proliferation relative to non-cleavable BMPS controls. FIG. 10B shows surface expression of CD25 (IL-2 receptor a chain) on activated CD8⁺ T cells following stimulation with the indicated SNA constructs (n=3). SNAs comprising hybrid linker-antigen peptides, specifically GQI-OVA₂₅₇₋₂₆₄, exhibited increased CD25 expression relative to comparator formulations. FIG. 10C shows expression of KLRG1 on CD8⁺ T cells following activation with the indicated SNA constructs (n=3). SNAs comprising hybrid linker-antigen peptides, specifically GQS-OVA₂₅₇₋₂₆₄ and GQI-OVA₂₅₇₋₂₆₄, increased the frequency of KLRG1⁺ effector phenotypes relative to comparator formulations. Statistical significance between groups was determined using one-way ANOVA with Dunnett's multiple comparison test. *P<0.05; **P<0.01; ****P<0.0001. Data are presented as mean±STDEV.

FIG. 11 depicts immune responses evaluating CD8⁺ T cell phenotypic analysis following short-duration immunization with SNA formulations comprising hybrid linker-antigen peptides compared to SNAs comprising redox-responsive linkers. FIG. 11A shows the immunization schedule. FIG. 11B show the frequency of cytotoxic CD8⁺ T cells within splenocytes across treatment groups (n=7-8). FIG. 11C shows the proportion of antigen-specific (Pentamer⁺) T cells within the CD8⁺ population (n=7-8). FIG. 11D shows the proportion of short-lived effector (KLRG1⁺/CD127⁻) phenotypes within the CD8⁺ T cell population in splenocytes (n=7-8). FIG. 11E shows the proportion of antigen-specific (Pentamer⁺) T cells within the short-lived effector (KLRG1⁺/CD127⁻) CD8⁺ T cell subset (n=7-8). FIG. 11F shows the proportion of effector-memory (CD44⁺/CD62L⁻/KLRG1⁻) phenotypes within the CD8⁺ T cell population in splenocytes (n=7-8). FIG. 11G shows the proportion of antigen-specific (Pentamer⁺) T cells within the effector-memory (CD44⁺/CD62L⁻/KLRG1⁻) CD8⁺ T cell subset (n=7-8). FIG. 11H shows cytotoxic activity of isolated CD8⁺ T cells against ovalbumin-expressing target cells (E.G7-OVA) at effector to target ratio of 12.5:1, measured by early apoptosis (Annexin V⁺/7-AAD⁻) and late apoptosis (Annexin V⁺/7-AAD⁺) (n=4). Statistical significance was determined by one-way Anova with Dunnett's multiple comparison test. **P<0.01, ***P<0.001,****P<0.0001. Data presented as mean±SEM.

FIG. 12 depicts in vivo antitumor activity following administration of SNA cancer vaccines comprising hybrid linker-antigen peptides compared to SNAs comprising redox-responsive linkers in the MC-38 tumor model. FIG. 12A shows the dosing schedule for treatment of MC-38 colon carcinoma tumors following tumor inoculation. FIG. 12B shows tumor growth curves for MC-38 tumors in mice treated with saline (PBS), CSS-Adpgk I SNA, or GQI-Adpgk I SNA. FIG. 12C shows quantitative comparison of tumor volumes among treatment groups at day 19 post-tumor inoculation. FIG. 12D shows Kaplan-Meier survival curves for each treatment group through day 33 post-tumor inoculation. Statistical significance for tumor growth comparisons was determined by one-way ANOVA with Dunnett's multiple comparison test. *P<0.05, ***P<0.001,****P<0.0001. Data are presented as mean±SEM, with n=7-8 per group.

FIG. 13 depicts in vivo antitumor activity following administration of SNA cancer vaccines comprising hybrid linker-antigen peptides compared to SNAs comprising redox-responsive linkers in the B16-F10 melanoma model as monotherapy. FIG. 13A shows the dosing schedule for treatment of B16-F10 tumors following tumor inoculation. FIG. 13B shows tumor growth curves for B16-F10 tumors in mice treated with saline (PBS), M27 SNA, or GQI-M27 SNA. FIG. 13C shows quantitative comparison of tumor volumes among treatment groups at day 17 post-tumor inoculation. FIG. 13D shows Kaplan-Meier survival curves for each treatment group through day 33 post-tumor inoculation. Statistical significance for tumor growth comparisons was determined by one-way ANOVA with Dunnett's multiple comparison test. *P<0.05, ***P<0.001,****P<0.0001. Data are presented as mean±SEM, with n=11-12 per group.

### DETAILED DESCRIPTION

The instant disclosure provides hybrid peptides, spherical nucleic acids (SNAs) comprising the hybrid peptides, and compositions comprising the hybrid peptides or the SNAs, and methods for using the aforementioned components to induce an immune response in a subject.

Spherical nucleic acids (SNAs), structures that comprise a spherical particle core densely functionalized with radially oriented DNA, are modular entities that can be designed with adjuvant DNA and paired with antigens to directly impact immune stimulation and vaccine function, respectively. Indeed, immunostimulatory SNAs incorporating cancer-relevant antigens strategically positioned on or within the SNA structure have shown promise as cancer vaccines.

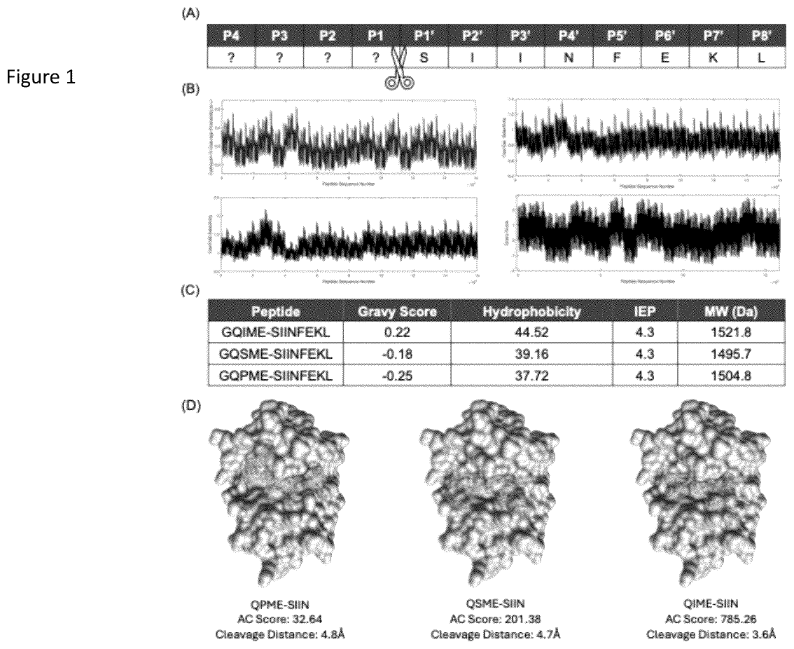

Current peptide-based linker chemistries are not optimized to facilitate the release of peptide antigens in their native, immunogenetic state for targeting the vacuolar cross-presentation pathway. For example, the previously established PMG-LP peptide, a peptide designed to be highly selective to cleavage by Cathepsin S,⁶ is designed to have a singular cleavage site between G-L. Thus, post-cleavage, an antigen peptide will be left with an additional LP motif on its N-terminus, which must be cleaved by aminopeptidases prior to successful docking to MHC class I complexes. An ideal peptide linker sequence would, upon proteolysis by Cathepsin S, allow for the release of antigen peptides in their native state and thus facilitate direct binding to MHC class I within endosomal structures. Disclosed herein is the finding that meticulous arrangement of amino acids on the N-terminus of known peptide antigens (FIG. 1A) revealed a design for a peptide sequence that facilitates cleavage of the peptide bond between the C-terminus of the linker sequence, and the N-terminus of a peptide antigen; thereby releasing the antigen peptide in its native state. It is contemplated that this design may be applied to peptide antigens independent of antigen sequence.

As used in this specification and the appended claims, the singular forms “a,” “an,” and “the” include plural reference unless the context clearly dictates otherwise. The articles “a” and “an” refer to one or more than one (for example, to at least one) of the grammatical object of the article.

All language such as “from,” “to,” “up to,” “at least,” “greater than,” “less than,” and the like include the number recited and refer to ranges which can subsequently be broken down into sub-ranges.

As used herein, an “immunostimulatory oligonucleotide” is an oligonucleotide that can stimulate (e.g., induce or enhance) an immune response. Typical examples of immunostimulatory oligonucleotides are CpG-motif containing oligonucleotides, single-stranded RNA oligonucleotides, double-stranded RNA oligonucleotides, and double-stranded DNA oligonucleotides. A “CpG-motif” is a cytosine-guanine dinucleotide sequence. In any of the aspects or embodiments of the disclosure, the immunostimulatory oligonucleotide is a toll-like receptor (TLR) agonist (e.g., a toll-like receptor 9 (TLR9) agonist). In various embodiments, the TLR agonist is a toll-like receptor 1 (TLR-1) agonist, toll-like receptor 2 (TLR-2) agonist, toll-like receptor 3 (TLR-3) agonist, toll-like receptor 4 (TLR-4) agonist, toll-like receptor 5 (TLR-5) agonist, toll-like receptor 6 (TLR-6) agonist, toll-like receptor 7 (TLR-7) agonist, toll-like receptor 8 (TLR-8) agonist, toll-like receptor 9 (TLR-9) agonist, toll-like receptor 10 (TLR-10) agonist, toll-like receptor 11 (TLR-11) agonist, toll-like receptor 12 (TLR-12) agonist, toll-like receptor 13 (TLR-13) agonist, or a combination thereof.

A “linker” as used herein is a structure that joins an antigen, or hybrid peptide to an oligonucleotide, as described herein. In some embodiments, the shell of oligonucleotides comprises a hybrid peptide and an antigen, wherein the hybrid peptide and the antigen are attached to separate oligonucleotides. In any of the aspects or embodiments of the disclosure, a linker is a non-cleavable linker (e.g., N-β-maleimidopropyl-oxysuccinimide ester (BMPS)), a traceless linker, a disulfide linker, or a peptide-based linker. A “Cathepsin-S Sensitive Linker” as used herein is an amino acid sequence attached to an antigen. In some embodiments, the Cathepsin-S sensitive linker containing antigen is linked to an oligonucleotide by a non-cleavable linker. A “lipid anchor” as used herein is a structure that joins an oligonucleotide to a nanoparticle core, wherein the nanoparticle core is a liposomal core or a lipid nanoparticle core.

A “subject” is a vertebrate organism. The subject can be a non-human mammal (e.g., a mouse, a rat, or a non-human primate), or the subject can be a human subject.

An “antigenic composition” is a composition suitable for administration to a human or animal subject that is capable of eliciting a specific immune response, e.g., against an antigen. Thus, an antigenic composition includes one or more antigens (for example, a tumor associated antigen, a tumor specific antigen, a neoantigen) or antigenic epitopes. In some embodiments, antigenic compositions are administered to elicit an immune response that protects the subject against symptoms or conditions induced by an antigen. In certain embodiments, the antigenic composition induces or boosts an immune response against cancer. In some embodiments, symptoms or disease caused by an antigen of the disclosure is prevented, reduced, or ameliorated by inhibiting expansion of cells associated with, e.g., a tumor. In some embodiments, symptoms or disease caused by an antigen of the disclosure is prevented, reduced, or ameliorated by inhibiting tumor growth.

An “antigen” is a molecule or molecular structure within a protein (e.g., a peptide derived from a protein). The presence of antigens in the body could be recognized by antigen presenting cells (APCs) and normally triggers an immune response.

An “immune response” is a response of a cell of the immune system, such as a B cell or T cell, to a stimulus, such as an antigen of the disclosure (e.g., formulated as an antigenic composition or a vaccine). An immune response can be a B cell response, which results in the production of antigen-specific antibodies. An immune response can also be a T cell response, such as a CD4⁺ T cell response or a CD8⁺ T cell response. As described herein, an immune response may be an enhanced CD4⁺ and/or CD8⁺ T cell response (relative to the CD4⁺ and/or CD8⁺ T cell response in the absence of exposure to an SNA of the disclosure) depending on the SNA that is utilized (e.g., administered). An immune response can be measured, for example, by an enzyme linked immunosorbent assay (ELISA), by T cell proliferation, or by measurement of the proportion of T cell subsets such as memory or activated cells.

As used herein, the term “about,” when used to modify a particular value or range, generally means within 20 percent, e.g., within 10 percent, 5 percent, 4 percent, 3 percent, 2 percent, or 1 percent of the stated value or range.

Unless otherwise stated, all ranges contemplated herein include both endpoints and all numbers between the endpoints. The use of “about” or “approximately” in connection with a range applies to both ends of the range. Thus, “about 20 to 30” is intended to cover “about 20 to about 30”, inclusive of at least the specified endpoints.

The terms “administering”, “administer”, “administration”, and the like, as used herein, refer to any mode of transferring, delivering, introducing, or transporting, for example, a SNA, a composition comprising a SNA, a pharmaceutical formulation, an antigenic composition, or a combination thereof to a subject in need of treatment with such an agent. Such modes include, but are not limited to, intravenous, intraarterial, intraperitoneal, intranasal, intrathecal, and subcutaneous administration.

The terms “polynucleotide” and “oligonucleotide” are interchangeable as used herein.

### MHC Class 1

Major histocompatibility complex (MHC) class I is a heterodimeric complex that presents peptides derived from full length proteins, referred to herein as antigens, to the adaptive immune system to prime an antigen-specific immune response. MHC class I proteins bind and present antigens generated via the degradation of intracellular proteins, and non-native peptides are recognized by circulating CD8⁺ cytotoxic T cells (Asano et al., Immunity, 34: 85-95, 2011).

Antigens are generally generated via the degradation of intracellular proteins in the cytosol for presentation on MHC class I, or via the degradation of extracellular proteins introduced into a cell via endocytosis or phagocytosis for cross-presentation on MHC class I. The classical pathway of extracellular antigen processing and ultimate presentation in MHC class I on the surface of an antigen presenting cell (APC), known as the endosome-to-cytosol cross-presentation pathway, begins with endocytosis or phagocytosis of an extracellular protein into an endosome, and preliminary processing of this protein by an endosomal protease (Mantegazza et al., Traffic, 14(2): 135-52, 2013). The products of that preliminary processing are exported to the cytosol, where these products are further processed by the proteasome into antigen precursors (Rodriguez et al., Nat Cell Biol, 1: 362-68, 1999). The antigen precursors are translocated to the endoplasmic reticulum (ER) for processing, and the processed antigens are loaded into an MHC class I, and the MHC class I-antigen complex is exported from the ER via the classical secretory pathway and is expressed on the plasma membrane of the cell (Kreer et al., Front Immunol, 2: 87, 2011). In addition, the non-classical pathway, known as the vacuolar cross-presentation pathway, begins with endocytosis or phagocytosis of an extracellular protein into an endosome, where it is directly processed into an antigen capable of binding to MHC class I (Shen et al., Immunity, 21: 155-165, 2004). The antigen is directly loaded into an MHC class I, and the MHC class I-antigen complex is translocated to the plasma membrane of the cell for presentation (Joffre et al., Nat Rev Immunol, 12: 557-569, 2012).

To fit the groove of most MHC class I molecules, antigenic peptides are generally understood to have a length of 8-10 residues (Jones, Curr Opin Immunol, 9: 75-79, 1997), although certain class I molecules can present peptides up to 11 residues (Rammensee et al., Immunogenetics, 41: 179-228, 1995). MHC class I molecules typically present only a small fraction of potential peptides, and even fewer peptides trigger a potent T cell response (Hearn et al., J Immunol, 183(9): 5526-36, 2009); antigen processing and ultimate epitope abundance at the cell surface are major factors underlying whether an antigen induces a T cell response (Restifo et al., J Immunol, 154: 4414-22, 1995).

### Hybrid Peptides, Antigens, and Cathepsin S-Sensitive Linkers

In some embodiments, the disclosure provides a hybrid peptide. In various aspects, the hybrid peptide comprises a Cathepsin S-sensitive linker attached to an antigen, wherein the Cathepsin S-sensitive linker attached to the antigen comprises a four amino acid sequence. In some aspects, the four amino acid sequence is Gln-X-Met-Glu, wherein X is an amino acid residue. In various aspects, the amino acid that comprises residue X is a hydrophobic amino acid. In various embodiments, the amino acid that comprises residue X can be selected from the group consisting of Pro, Ser, and lie. In further aspects, the Cathepsin S-sensitive linker further comprises a Gly amino acid residue at its N-terminus. In some embodiments, the Cathepsin-S sensitive linker is attached to the N-terminus of the antigen.

In various aspects, the disclosure provides a spherical nucleic acid (SNA) comprising or consisting of (a) a nanoparticle core; (b) a shell of oligonucleotides comprising one or more oligonucleotides attached to the nanoparticle core; and (c) a hybrid peptide attached to one or more oligonucleotides in the shell of oligonucleotides, wherein the hybrid peptide comprises a Cathepsin S-sensitive linker attached to an antigen. In further aspects, the disclosure provides a SNA comprising or consisting of (a) a nanoparticle core; (b) a shell of oligonucleotides comprising one or more oligonucleotides attached to the nanoparticle core; and (c) a hybrid peptide attached to one or more oligonucleotides in the shell of oligonucleotides, wherein the hybrid peptide comprises a Cathepsin S-sensitive linker attached to an antigen, configured as follows: nanoparticle core----oligonucleotide----additional linker-----Cathepsin S-sensitive linker----antigen. In various aspects, the disclosure provides a hybrid peptide, wherein the hybrid peptide comprises a Cathepsin S-sensitive linker attached to an antigen. In various aspects, the Cathepsin S-sensitive linker is cleavable and traceless. Traceless linkers (such as the Cathepsin S-sensitive linker) can be used to maintain the unique properties of SNA architecture—for example and without limitation, efficient cellular uptake and TLR activation—without sacrificing the biological efficacy of the delivered antigen. This property stems from the ability of the traceless linker to release the antigen in its native state. In some embodiments, traceless linkers can be used to conjugate antigens to SNAs.

In some embodiments, the structure of the hybrid peptide is represented by the schematic presented in FIG. 1A, wherein the amino acid residues of the antigen comprise or consist of residues P1′ to P9′, P1′ to P10′, or P1′ to P11′, and the amino acid residues of the Cathepsin S-sensitive linker comprise P4 to P1 or P5 to P1. In some embodiments, the hybrid peptide is cleaved between the P1 and the P1′ position. In some embodiments, the substrate-binding pocket of Cathepsin S is S4-S1/S1′-S4′. In various embodiments, “P” corresponds to the substrate amino acids, and “S” refers to the enzyme substrate binding pocket.

In still further aspects, the disclosure provides spherical nucleic acids (SNAs) comprising or consisting of (a) a nanoparticle core; (b) a shell of oligonucleotides comprising one or more oligonucleotides attached to the nanoparticle core; and (c) a hybrid peptide comprising a Cathepsin S-sensitive linker and an antigen. In some embodiments, a SNA of the disclosure comprises a hybrid peptide that comprises an antigen and a Cathepsin S-sensitive linker. In various embodiments, the antigen comprises 8-11 amino acids. In various embodiments, the antigen is a peptide. In some embodiments, the antigen is a cancer-related antigen. In further embodiments, the antigen is a tumor associated antigen, a tumor specific antigen, a neoantigen, or a combination thereof. In various embodiments, the cancer is breast cancer, peritoneum cancer, cervical cancer, colon cancer, rectal cancer, esophageal cancer, eye cancer, liver cancer, pancreatic cancer, larynx cancer, lung cancer, skin cancer, ovarian cancer, prostate cancer, stomach cancer, testicular cancer, thyroid cancer, brain cancer, or a combination thereof. In various embodiments, the antigen is or comprises ovalbumin, OVA1, MSLN, P53, Ras, mutant IDH1 (IDH1R132H), a melanoma related antigen, a HPV related antigen, a prostate cancer related antigen, a glioblastoma antigen, a grade IV astrocytoma antigen, an ovarian cancer related antigen, a breast cancer related antigen, a hepatocellular carcinoma related antigen, a bowel cancer related antigen, or human papillomavirus (HPV) E7 nuclear protein. In some embodiments, the antigen is or comprises OVA-associated antigen SIINFEKL (SEQ ID NO: 7), human melanoma antigen gp100 KVPRNQDWL (SEQ ID NO: 8), murine H2Db antigen TAPDNLGYA (SEQ ID NO: 9), and the M27 melanoma neoantigen LCPGNKYEM (SEQ ID NO: 10).

In some embodiments, the hybrid peptide comprising an antigen is attached to an oligonucleotide in the shell of oligonucleotides. In some embodiments, the hybrid peptide is attached to each oligonucleotide in the shell of oligonucleotides. In some embodiments, the hybrid peptide comprising an antigen is attached to a double stranded oligonucleotide in the shell of oligonucleotides. In some embodiments, the hybrid peptide is attached to the 3′ end of an oligonucleotide in the shell of oligonucleotides. In some embodiments, the hybrid peptide comprising an antigen is attached to an oligonucleotide via an additional linker. In various aspects, the additional linker is a non-cleavable linker, a cleavable but non-traceless linker, a cleavable and traceless linker, a disulfide linker, or a peptide-based linker. In some aspects, the non-cleavable linker is N-β-maleimidopropyl-oxysuccinimide ester (BMPS). In some aspects, the cleavable traceless linker is SDEC. In some embodiments, a cleavable linker is sensitive to (and is cleaved in response to) a reducing agent (e.g., glutathione (GSH), dithiothreitol (DTT)) or a reducing environment (e.g., inside a cell). In various embodiments, a cleavable linker is sensitive to (and is cleaved in response to) various chemical stimuli such as, for example, acidity (e.g., low pH), an enzyme (e.g., peptidase), light (e.g., NIR laser), and/or hydrolysis. In some embodiments, a traceless linker releases an antigen in its native state. In various embodiments a linker is cleavable and non-traceless, and upon cleavage will leave a chemical pendant or one or more amino acids attached to the antigen. In some embodiments, the hybrid peptide is attached to the surface of the nanoparticle core, the hybrid peptide is attached to an oligonucleotide in the shell of oligonucleotides, or both. (see, e.g., U.S. Patent Application Publication No. 2020/0384104, incorporated herein by reference in its entirety).

In various aspects, the disclosure provides spherical nucleic acids (SNAs) comprising or consisting of (a) a nanoparticle core (b) a shell of oligonucleotides comprising one or more oligonucleotides attached to the nanoparticle core; (c) a hybrid peptide comprising a Cathepsin S-sensitive linker and an antigen; and (d) another antigen, wherein the antigens are different antigens relative to each other. In some embodiments, “different antigens” are antigens that share about or less than about 80% sequence identity. In various embodiments, “different antigens” are antigens that share about or less than about 75%, 60%, 65%, 50%, 45%, 40%, 35%, 30%, 25%, 20%, 15%, 10%, or 5% sequence identity. In some embodiments, either antigen comprises any of the peptides, cancer-related antigens, tumor associate antigens, neo-antigens, and compositions thereof that the antigen comprises. In some embodiments, the SNAs comprise two or more antigens. In some aspects the two or more antigens are different antigens relative to each other. In some embodiments, the two or more antigens are the same antigen.

In some aspects and embodiments, one or more oligonucleotides in the shell of oligonucleotides that is attached to the nanoparticle core of a SNA comprise a spacer. “Spacer” as used herein means a moiety that serves to increase distance between the nanoparticle core and the oligonucleotide, or to increase distance between individual oligonucleotides when attached to the nanoparticle core in multiple copies, or to improve the synthesis of the SNA. Thus, spacers are contemplated being located between an oligonucleotide and the nanoparticle core. In some aspects, the spacer when present is an organic moiety. In some aspects, the spacer is a polymer, including but not limited to a water-soluble polymer, a nucleic acid, a polypeptide, an oligosaccharide, a carbohydrate, a lipid, an ethylglycol, or a combination thereof. In any of the aspects or embodiments of the disclosure, the spacer is an oligo(ethylene glycol)-based spacer. In various embodiments, an oligonucleotide comprises 1, 2, 3, 4, 5, or more spacer (e.g., Spacer-18 (hexaethyleneglycol)) moieties. In further embodiments, the spacer is an alkane-based spacer (e.g., C12). In some embodiments, the spacer is an oligonucleotide spacer (e.g., T5). An oligonucleotide spacer may have any sequence that does not interfere with the ability of the oligonucleotide to perform an intended function (e.g., inhibit gene expression). In certain aspects, the bases of the oligonucleotide spacer are all adenylic acids, all thymidylic acids, all cytidylic acids, all guanylic acids, all uridylic acids, or all some other modified base. In various embodiments, the length of the spacer is or is equivalent to at least about 2 nucleotides, at least about 3 nucleotides, at least about 4 nucleotides, at least about 5 nucleotides, 5-10 nucleotides, 10 nucleotides, 20 nucleotides, 10-30 nucleotides, or even greater than 30 nucleotides. In various aspect, the spacer is a polyethylene glycol (PEG) spacer.

In some aspects, the spacer when present is an organic moiety. In some aspects, the spacer is a polymer, including but not limited to a water-soluble polymer, a nucleic acid, a polypeptide, an oligosaccharide, a carbohydrate, a lipid, an ethylglycol, or a combination thereof. In any of the aspects or embodiments of the disclosure, the spacer is an oligo(ethylene glycol)-based spacer. In various embodiments, an oligonucleotide comprises 1, 2, 3, 4, 5, or more spacer (e.g., Spacer-18 (hexaethyleneglycol)) moieties. In further embodiments, the spacer is an alkane-based spacer (e.g., C12). In some embodiments, the spacer is an oligonucleotide spacer (e.g., T5). An oligonucleotide spacer may have any sequence that does not interfere with the ability of the oligonucleotides to become bound to the liposomal core or to a target. In certain aspects, the bases of the oligonucleotide spacer are all adenylic acids, all thymidylic acids, all cytidylic acids, all guanylic acids, all uridylic acids, or all some other modified base. In various embodiments, the length of the spacer is or is equivalent to at least about 2 nucleotides, at least about 3 nucleotides, at least about 4 nucleotides, at least about 5 nucleotides, 5-10 nucleotides, 10 nucleotides, 10-30 nucleotides, or even greater than 30 nucleotides.

### Cleavage Probability Value

Cysteine cathepsins, including Cathepsin S, play crucial roles in various physiological and pathological processes, particularly in cellular homeostasis and immune cell modulation. They share commonalities in their substrate specificity due to their highly conserved catalytic triad containing Cys, His, and Asn. To clarify Cathepsin-specific substrate recognition, substrate libraries, including the Proteomic Identification of Protease Cleavage Sites (PICS) database, have been generated for simultaneous profiling of prime and non-prime specificity of Cathepsins (Biniossek et al., J Proteome Res, 10(12): 5363-73, 2011). The aforementioned Cathepsin S PICS data was used herein to screen the likelihood of proteolysis for every possible Cathepsin S-sensitive linker spanning the P4-P1 position. Specifically, a “cleavage probability value” (Cleav_{PROB}) was calculated by summation of the PICS normalized positional occurrence for each amino acid (Pₐₐ) throughout the peptide sequence from P4-P4′. This summed value was normalized to the difference between the summed maximum (P_{MAX}) and summed minimum (P_{Min}) achievable values from the P4-P4′ positions within the database. The specific equation to calculate “cleavage probability” is provided below:

$${Cleav}_{P R 0 B} = \frac{{\sum }_{P 4}^{P {4}^{′}} {P}_{a a}}{( {P}_{M A X} - {P}_{MIN} )}$$

$$P = Normalized Positional Occurrence$$

In various embodiments, the disclosure provides an SNA comprising a hybrid peptide and/or a hybrid peptide, wherein the cleavage probability value of the hybrid peptide is at least 0.3, 0.4, 0.5, or 0.6. In various embodiments, the cleavage probability value of the hybrid peptide is at least 0.395.

### Spherical Nucleic Acids

As described herein, spherical nucleic acids (SNAs) are a unique class of nanomaterials comprising a spherical or substantially spherical nanoparticle core functionalized with a highly oriented oligonucleotide shell that is attached to the external surface of the nanoparticle core. SNAs are highly modular and chemically well-defined architectures produced by chemical synthesis and programmed assembly, allowing for their rapid modification to incorporate novel antigens. This rational vaccinology approach is in stark contrast to the classical approach of vaccination and enables the generation of superior protective immune responses, as well as methods to rapidly produce vaccines at scale. More specifically, in some aspects the disclosure provides a spherical nucleic acid (SNA) comprising or consisting of (a) a nanoparticle core; (b) a shell of oligonucleotides comprising one or more oligonucleotides attached to the nanoparticle core; and (c) a hybrid peptide attached to one or more oligonucleotides in the shell of oligonucleotides. In various embodiments, the hybrid peptide is a Cathepsin S-sensitive linker attached to an antigen. In further aspects, the disclosure provides a spherical nucleic acid (SNA) comprising or consisting of (a) a nanoparticle core; (b) a shell of oligonucleotides comprising one or more oligonucleotides attached to the nanoparticle core; and (c) a hybrid peptide attached to one or more oligonucleotides in the shell of oligonucleotides, wherein the Cathepsin-S sensitive linker is a four amino acid sequences. In still further aspects, the disclosure provides a spherical nucleic acid (SNA) comprising or consisting of (a) a nanoparticle core; (b) a shell of oligonucleotides comprising one or more oligonucleotides attached to the nanoparticle core; and (c) a hybrid peptide attached to one or more oligonucleotides in the shell of oligonucleotides, wherein the Cathepsin S-sensitive linker attached to the antigen comprises Gln-X-Met-Glu. In various aspects, X is an amino acid. In various aspects, the amino acid that comprises residue X is a hydrophobic amino acid. In various embodiments, the amino acid that comprises residue X can be selected from the group consisting of Pro, Ser, and lie.

In further aspects, the disclosure provides a spherical nucleic acid (SNA) comprising or consisting of (a) a nanoparticle core; (b) a shell of oligonucleotides comprising one or more oligonucleotides attached to the nanoparticle core, wherein one or more oligonucleotides in the shell of oligonucleotides comprises a double-stranded oligonucleotide; and (c) a hybrid peptide attached to one or more oligonucleotides in the shell of oligonucleotides. In various aspects, one strand of the double-stranded oligonucleotide in the shell of oligonucleotides comprises the nucleotide sequence of SEQ ID NO: 1, SEQ ID NO: 2, or SEQ ID NO: 3. In various aspects, one strand of the double-stranded oligonucleotide in the shell of oligonucleotides comprises the nucleotide sequence of SEQ ID NO: 4, SEQ ID NO: 5, or SEQ ID NO: 6. In still further aspects, the disclosure provides a spherical nucleic acid (SNA) comprising or consisting of (a) a nanoparticle core; (b) a shell of oligonucleotides comprising one or more oligonucleotides attached to the nanoparticle core, wherein the shell of oligonucleotides comprises DNA, RNA, or a combination thereof; and (c) a hybrid peptide attached to one or more oligonucleotides in the shell of oligonucleotides. In various aspects, the disclosure provides a spherical nucleic acid (SNA) comprising or consisting of (a) a nanoparticle core; (b) a shell of oligonucleotides comprising one or more oligonucleotides attached to the nanoparticle core, wherein the shell of oligonucleotides comprises a single-stranded DNA, a double-stranded DNA, a single-stranded RNA, a double-stranded RNA, or a combination thereof; and (c) a hybrid peptide attached to one or more oligonucleotides in the shell of oligonucleotides. In still other aspects, the disclosure provides a spherical nucleic acid (SNA) comprising or consisting of (a) a nanoparticle core; (b) a shell of oligonucleotides comprising one or more oligonucleotides attached to the nanoparticle core, wherein the shell of oligonucleotides comprises an immunostimulatory oligonucleotide; and (c) a hybrid peptide attached to one or more oligonucleotides in the shell of oligonucleotides. In various aspects, each oligonucleotide in the shell of oligonucleotides is an immunostimulatory oligonucleotide. In various aspects, the immunostimulatory oligonucleotide is a CpG-motif containing oligonucleotide. In various aspects, the immunostimulatory oligonucleotide is a TLR agonist. In various aspects, the disclosure provides a spherical nucleic acid (SNA) comprising or consisting of (a) a nanoparticle core; (b) a shell of oligonucleotides comprising one or more oligonucleotides attached to a nanoparticle core, wherein one or more oligonucleotides in the shell of oligonucleotides comprises SEQ ID NO: 4, and; (c) a hybrid peptide attached to the 3′ end of one or more oligonucleotides comprising SEQ ID NO: 4. In various aspects, the disclosure provides a spherical nucleic acid (SNA) comprising or consisting of (a) a nanoparticle core; (b) a shell of oligonucleotides comprising one or more oligonucleotides attached to a nanoparticle core, wherein each oligonucleotide in the shell of oligonucleotides comprises SEQ ID NO: 4, and; (c) a hybrid peptide attached to the 3′ end of each of the one or more oligonucleotides comprising SEQ ID NO: 4.

The nanoparticle core is, in various embodiments, a lipid-based core, a polymer core, a protein core, or an inorganic core. In some embodiments, the lipid-based core is a liposomal core. In some embodiments, the lipid-based core is a lipid nanoparticle (LNP) core. Thus, the disclosure provides liposomal SNAs (LSNAs), lipid nanoparticle SNAs (LNP-SNAs), polymer SNAs, protein core SNAs (ProSNAs), and inorganic core SNAs.

The SNA architecture provides many chemical handles for the rapid synthesis and evaluation of modular and programmable vaccines. In turn, this enables one to tune several important vaccine design parameters, including spatial arrangement, presentation, and stoichiometric ratio of adjuvant and antigen (Bachmann et al., Nature Reviews Immunology, 10 (11), 787-796, 2010). Moreover, the inclusion of a plurality of antigens from the same or different peptides and proteins can be rapidly evaluated in the SNA format, allowing for the rapid development of new vaccines to counter the threat of antigenic shift and evolution in cancer.

The composition of the shell of oligonucleotides is also highly modular, and can variously comprise DNA oligonucleotides, RNA oligonucleotides, modified forms thereof, or a combination thereof. The DNA oligonucleotides may be single-stranded, double-stranded, or a combination thereof. The RNA oligonucleotides may be single-stranded, double-stranded, or a combination thereof. The spherical architecture of the polynucleotide shell confers unique advantages over traditional nucleic acid delivery methods, including entry into nearly all cells independent of transfection agents and resistance to nuclease degradation. Furthermore, SNAs can penetrate biological barriers, including the blood-brain (see, e.g., U.S. Patent Application Publication No. 2015/0031745, incorporated by reference herein in its entirety) and blood-tumor barriers as well as the epidermis (see, e.g., U.S. Patent Application Publication No. 2010/0233270, incorporated by reference herein in its entirety).

In some aspects, a SNA of the disclosure comprises a nanoparticle core, a shell of oligonucleotides comprising DNA oligonucleotides and RNA oligonucleotides, or a combination thereof. In some embodiments, the shell of oligonucleotides comprises or consists of one or more immunostimulatory oligonucleotides. In further embodiments, the one or more immunostimulatory oligonucleotides is a toll-like receptor (TLR) agonist. In various embodiments, the toll-like receptor (TLR) agonist is a human toll-like receptor (TLR) agonist. In some embodiments, the TLR agonist is toll-like receptor 3 (TLR3) agonist, toll-like receptor 4 (TLR4) agonist, toll-like receptor 7 (TLR7) agonist, toll-like receptor 8 (TLR8) agonist, toll-like receptor 9 (TLR9) agonist, or a combination thereof. In still further embodiments the TLR agonist is a toll-like receptor 9 (TLR9) agonist. In some embodiments, the TLR9 agonist is CpG 1826 (murine TLR9 agonist)(5′-TCC ATG ACG TTC CTG ACG TT-3′: SEQ ID NO: 1), CpG 1018 (murine and human TLR9 agonist)(5′-TGA CTG TGA ACG TTC GAG ATG A-3′: SEQ ID NO: 2), CpG 2006 (human TLR agonist) (5′-TCG TCG TTT ⁻ embodiments, the TLR9 agonist is complementary CpG 1826 (SEQ ID NO: 4), complementary CpG 1018 (SEQ ID NO: 5), complementary CpG 2006 (SEQ ID NO: 6).

In general, nanoparticles can range in size from about 10 nm to about 150 nm in diameter, about 10 nm to about 140 nm in diameter, about 10 nm to about 130 nm in diameter, about 10 nm to about 120 nm in diameter, about 10 nm to about 110 nm in diameter, about 10 nm to about 100 nm in diameter, about 10 nm to about 90 nm in diameter, about 10 nm to about 80 nm in diameter, about 10 nm to about 70 nm in diameter, about 10 nm to about 60 nm in diameter, about 10 nm to about 50 nm in diameter, about 10 nm to about 40 nm in diameter, about 10 nm to about 30 nm in diameter, or about 10 nm to about 20 nm in diameter. In other aspects, the disclosure provides a plurality of nanoparticles, each nanoparticle having a substantially spherical geometry comprising a shell of oligonucleotides attached thereto, wherein one or more of the oligonucleotide in the shell is an immunostimulatory oligonucleotide (e.g., a Toll-Like Receptor (TLR) agonist), and wherein a hybrid peptide is attached to one or more oligonucleotides in the shell of oligonucleotides on the surface of the nanoparticle core. In these aspects, the size of the plurality of nanoparticles is from about 10 nm to about 150 nm (mean diameter), about 10 nm to about 140 nm in mean diameter, about 10 nm to about 130 nm in mean diameter, about 10 nm to about 120 nm in mean diameter, about 10 nm to about 110 nm in mean diameter, about 10 nm to about 100 nm in mean diameter, about 10 nm to about 90 nm in mean diameter, about 10 nm to about 80 nm in mean diameter, about 10 nm to about 70 nm in mean diameter, about 10 nm to about 60 nm in mean diameter, about 10 nm to about 50 nm in mean diameter, about 10 nm to about 40 nm in mean diameter, about 10 nm to about 30 nm in mean diameter, or about 10 nm to about 20 nm in mean diameter. In some embodiments, the diameter (or mean diameter for a plurality of nanoparticles) of the nanoparticles is from about 10 nm to about 150 nm, from about 30 to about 100 nm, or from about 40 to about 80 nm. In some embodiments, the size of the nanoparticles used in a method varies as required by their particular use or application. The variation of size is advantageously used to optimize, for example, the amount of surface area to which oligonucleotides can be attached. In further embodiments, a plurality of SNAs (e.g., LSNAs, LNP-SNAs, polymer SNAs, ProSNAs, or inorganic SNAs) is produced and the SNAs in the plurality have a mean diameter of less than or equal to about 150 nanometers (e.g., about 10 nanometers to about 150 nanometers), or less than or equal to about 100 nanometers (e.g., about 10 nanometers to about 100 nanometers, or less than or equal to about 80 nanometers (e.g., about 10 nanometers to about 80 nanometers). In further embodiments, the nanoparticles in the plurality created by a method of the disclosure have a diameter or mean diameter of less than or equal to about 20 nanometers, or less than or equal to about 25 nanometers, or less than or equal to about 30 nanometers, or less than or equal to about 35 nanometers, or less than or equal to about 40 nanometers, or less than or equal to about 45 nanometers, or less than or equal to about 50 nanometers, or less than or equal to about 55 nanometers, or less than or equal to about 60 nanometers, or less than or equal to about 65 nanometers, or less than or equal to about 70 nanometers, or less than or equal to about 75 nanometers, or less than or equal to about 80 nanometers, or less than or equal to about 85 nanometers, or less than or equal to about 90 nanometers, or less than or equal to about 95 nanometers, or less than or equal to about 100 nanometers, or less than or equal to about 100 nanometers, or less than or equal to about 120 nanometers, or less than or equal to about 130 nanometers, or less than or equal to about 140 nanometers, or less than or equal to about 150 nanometers. It will be understood that the foregoing diameters of nanoparticles can apply to the diameter of the nanoparticle itself or to the diameter of the SNA (i.e., nanoparticle and oligonucleotides associated therewith).

### Liposomal Spherical Nucleic Acids (LSNAs)

In some aspects, the disclosure provides SNAs comprising a lipid-based core. In some embodiments, the lipid-based core is a liposomal core. Liposomal cores of the disclosure have at least a substantially spherical geometry, an internal side and an external side, and comprise a lipid bilayer. A liposomal spherical nucleic acid (LSNA) comprises a liposomal core, and a shell of oligonucleotides attached to the external side of the liposomal core. The lipid bilayer comprises, in various embodiments, a plurality of lipid groups. The plurality of lipid groups comprises, in various embodiments, a lipid from the phosphatidylcholine, phosphatidylglycerol, and/or phosphatidylethanolamine families of lipids. While not meant to be limiting, in various embodiments the lipid is 1,2-dioleoyl-sn-glycero-3-phosphocholine (DOPC), 1,2-dimyristoyl-sn-phosphatidylcholine (DMPC), 1-palmitoyl-2-oleoyl-sn-phosphatidylcholine (POPC), 1,2-distearoyl-sn-glycero-3-phosph′-(1′-rac-glycerol) (DSPG), 1,2-dioleoyl-sn-glycero-3-phosph′-(1′-rac-glycerol) (DOPG), 1,2-distearoyl-sn-glycero-3-phosphocholine (DSPC), 1,2-dipalmitoyl-sn-glycero-3-phosphocholine (DPPC), 1,2-di-(9Z-octadecenoyl)-sn-glycero-3-phosphoethanolamine (DOPE), 1,2-dihexadecanoyl-sn-glycero-3-phosphoethanolamine (DPPE), monophosphoryl Lipid A (MPLA), or a combination thereof.

Liposomes are spherical, self-closed structures in a varying size range comprising one or several hydrophobic lipid bilayers with a hydrophilic core. The diameter of these lipid based carriers range from 0.15-1 micrometers, which is significantly higher than an effective therapeutic range of 20-100 nanometers. Liposomes termed small unilamellar vesicles (SUVs), can be synthesized in the 20-50 nanometer size range, but encounter challenges such as instability and aggregation leading to inter-particle fusion. This inter-particle fusion limits the use of SUVs in therapeutics. Liposomal spherical nucleic acids (LSNAs) comprise a liposomal core, a shell of oligonucleotides attached to the external surface of the liposomal core, and a hybrid peptide attached to one or more oligonucleotides in the shell of oligonucleotides. Cathepsin S-sensitive linkers and antigens contemplated for use according to the disclosure are further described below.

Liposomal particles, for example as disclosed in International Patent Application No. PCT/US2014/068429 (incorporated by reference herein in its entirety) are therefore provided by the disclosure. Liposomal particles of the disclosure have at least a substantially spherical geometry, an internal side and an external side, and comprise a plurality of lipid groups. In various embodiments, the plurality of lipid groups comprises a lipid selected from the group consisting of the phosphatidylcholine, phosphatidylglycerol, and phosphatidylethanolamine families of lipids. Lipids contemplated by the disclosure include, without limitation, 1,2-dioleoyl-sn-glycero-3-phosphocholine (DOPC), 1,2-dimyristoyl-sn-phosphatidylcholine (DMPC), 1-palmitoyl-2-oleoyl-sn-phosphatidylcholine (POPC), 1,2-distearoyl-sn-glycero-3-phosph′-(1′-rac-glycerol) (DSPG), 1,2-dioleoyl-sn-glycero-3-phosph′-(1′-rac-glycerol) (DOPG), 1,2-distearoyl-sn-glycero-3-phosphocholine (DSPC), 1,2-dipalmitoyl-sn-glycero-3-phosphocholine (DPPC), 1,2-di-(9Z-octadecenoyl)-sn-glycero-3-phosphoethanolamine (DOPE), 1,2-dihexadecanoyl-sn-glycero-3-phosphoethanolamine (DPPE), 1,2-dioleoyl-sn-glycero-3-phosph′-(1′-rac-glycerol) (DOPG), 1,2-distearoyl-sn-glycero-3-phosphoethanolamine (DSPE), cardiolipin, lipid A, monophosphoryl Lipid A (MPLA), or a combination thereof.

In various embodiments, at least one oligonucleotide in the shell of oligonucleotides is attached to the exterior of the liposomal core through a lipid anchor group. In further embodiments, the lipid anchor group is attached to the 5′ end or the 3′ end of the at least one oligonucleotide. In still further embodiments, at least one oligonucleotide in the shell of oligonucleotides is attached to the exterior of a liposomal core via a covalent attachment of the oligonucleotide to a lipid-polyethylene glycol (lipid-PEG) conjugate. In various embodiments, 10%, 20%, 30%, 40%, 50%, 60%, 70%, 80%, 90%, or 100% of the oligonucleotides in the shell of oligonucleotides are covalently attached to the exterior of the liposomal core through the lipid-PEG conjugate. In various embodiments, at least one of the oligonucleotides in the shell of oligonucleotides is an oligonucleotide-lipid conjugate containing a lipid anchor group, wherein said lipid anchor group is adsorbed into the lipid bilayer, if the nanoparticle core is a liposomal core. In some embodiments, one or more of the oligonucleotides in the shell of oligonucleotides is an oligonucleotide-lipid conjugate containing a lipid anchor group, wherein said lipid anchor group is adsorbed into the lipid bilayer. In various embodiments, 10%, 20%, 30%, 40%, 50%, 60%, 70%, 80%, 90% or 100% of the oligonucleotides in the shell of oligonucleotides is attached (e.g., adsorbed) to the exterior of the liposomal core through a lipid anchor group. The lipid anchor group comprises, in various embodiments, tocopherol, palmitoyl, dipalmitoyl, stearyl, distearyl, cholesterol, or dodecane anchor (C12)9.

With respect to the surface density of oligonucleotides on the surface of a LSNA of the disclosure, it is contemplated that a LSNA as described herein comprises from about 1 to about 400 oligonucleotides on its surface. In various embodiments, a LSNA comprises from about 10 to about 100, or from 10 to about 90, or from about 10 to about 80, or from about 10 to about 70, or from about 10 to about 60, or from about 10 to about 50, or from about 10 to about 40, or from about 10 to about 30, or from about 10 to about 20, or from about 50 to about 100, or from about 60 to about 100, or from about 70 to about 100, or from about 80 to about 100, or from about 90 to about 100, or from about 75 to about 200, or from about 75 to about 150, or from about 100 to about 200, or from about 150 to about 200 oligonucleotides on its surface. In further embodiments, a LSNA comprises or consists of about, at least about, or less than about 5, 10, 20, 30, 40, 50, 60, 70, 75, 80, 90, 100, 150, 160, 170, 180, 190, 200, 250, 300, 350, or 400 oligonucleotides on its surface. In some embodiments, a LSNA comprises or consists of 70 oligonucleotides on its surface. In some embodiments, a LSNA comprises or consists of 75 oligonucleotides on its surface. Additional surface densities for SNAs are described herein below.

Methods of making a LSNA are generally known (see, e.g., Wang, S.; Qin, L.; Yamankurt, G.; Skakuj, K.; Huang, Z.; Chen, P.-C.; Dominguez, D.; Lee, A.; Zhang, B.; Mirkin, C. A. Rational Vaccinology with Spherical Nucleic Acids. Proc. Natl. Acad. Sci. 2019, 116 (21), 10473-10481, incorporated by reference herein in its entirety).

### Lipid Nanoparticle Spherical Nucleic Acids (LNP-SNAs)

In some aspects, the disclosure provides SNAs comprising a lipid-based core. In some embodiments, the lipid-based core is a lipid nanoparticle (LNP) core. Lipid nanoparticle spherical nucleic acids (LNP-SNAs) are comprised of a lipid nanoparticle core decorated with a shell of oligonucleotides. The lipid nanoparticle core comprises a hybrid peptide, an ionizable lipid, a phospholipid, a sterol, and a lipid-polyethylene glycol (lipid-PEG) conjugate. Hybrid peptides contemplated for use according to the disclosure are further described herein below. The shell of oligonucleotides is attached to the lipid nanoparticle core via a lipid anchor, and in many of the aspects or embodiments of the disclosure comprises one or more immunostimulatory oligonucleotides. The spherical architecture of the oligonucleotide shell confers unique advantages over traditional nucleic acid delivery methods, including entry into nearly all cells independent of transfection agents, resistance to nuclease degradation, sequence-based function, targeting, and diagnostics.

In some embodiments, the ionizable lipid is dilinoleylmethyl-4-dimethylaminobutyrate (DLin-MC3-DMA), 2,2-Dilinoleyl-4-dimethylaminoethyl-[1,3]-dioxolane (DLin-KC2-DMA), C12-200, 1,2-dioleoyl-3-dimethylammonium-propane (DODAP), similar lipid/lipidoid structures, or a combination thereof. In some embodiments, the phospholipid is 1,2-distearoyl-sn-glycero-3-phosphocholine (DSPC), 1,2-Dihexadecanoyl phosphatidylcholine (DPPC), 1,2-dioleoyl-sn-glycero-3-phosphocholine (DOPC), 1,2-dioleoyl-sn-glycero-3-phosphoethanolamine (DOPE), monophosphoryl Lipid A (MPLA), or a combination thereof. In further embodiments, the sterol is 3β-Hydroxycholest-5-ene (Cholesterol), 9,10-Secocholesta-5,7,10(19)-trien-3β-ol (Vitamin D3), 9,10-Secoergosta-5,7,10(19),22-tetraen-3β-ol (Vitamin D2), Calcipotriol, 24-Ethyl-5,22-cholestadien-3β-ol (Stigmasterol), 22,23-Dihydrostigmasterol (β-Sitosterol), 3,28-Dihydroxy-lupeol (Betulin), Lupeol, Ursolic acid, Oleanolic acid, 24α-Methylcholesterol (Campesterol), 24-Ethylcholesta-5,24(28)E-dien-3β-ol (Fucosterol), 24-Methylcholesta-5,22-dien-3β-ol (Brassicasterol), 24-Methylcholesta-5,7,22-trien-3β-ol (Ergosterol), 9,11-Dehydroergosterol, Daucosterol, or any of the foregoing sterols modified with one or more amino acids. In some embodiments, the lipid-polyethylene glycol (lipid-PEG) conjugate comprises 2000 Dalton (Da) polyethylene glycol. In further embodiments, the lipid-polyethylene glycol (lipid-PEG) conjugate is lipid-PEG-maleimide. In still further embodiments, the lipid-PEG-maleimide is 1,2-dipalmitoryl-sn-glycero-3-phosphoethanolamine (DPPE) conjugated to 2000 Da polyethylene glycol maleimide, 1,2-dimyristoyl-sn-glycero-3-phosphoethanolamine (DMPE) conjugated to 2000 Da polyethylene glycol maleimide, or a combination thereof.

In various embodiments, at least one oligonucleotide in the shell of oligonucleotides is attached to the exterior of the lipid nanoparticle core through a lipid anchor group. In further embodiments, the lipid anchor group is attached to the 5′ end or the 3′ end of the at least one oligonucleotide. In still further embodiments, at least one oligonucleotide in the shell of oligonucleotides is attached to the exterior of a lipid nanoparticle core via a covalent attachment of the oligonucleotide to a lipid-polyethylene glycol (lipid-PEG) conjugate. In various embodiments, 10%, 20%, 30%, 40%, 50%, 60%, 70%, 80%, 90%, or 100% of the oligonucleotides in the shell of oligonucleotides are covalently attached to the exterior of the lipid nanoparticle core through the lipid-PEG conjugate. In various embodiments, at least one of the oligonucleotides in the shell of oligonucleotides is an oligonucleotide-lipid conjugate containing a lipid anchor group. In some embodiments, one or more of the oligonucleotides in the shell of oligonucleotides is an oligonucleotide-lipid conjugate containing a lipid anchor group. The lipid anchor group comprises, in various embodiments, tocopherol, palmitoyl, dipalmitoyl, stearyl, distearyl, cholesterol, or dodecane anchor (C12)9.

### Polymer Core Spherical Nucleic Acids

In some embodiments, the nanoparticle core is a polymer-based particle (e.g., a poly (lactic-co-glycolic acid (PLGA) particle). Nanoparticle polymers include polystyrene, silicone rubber, polycarbonate, polyurethanes, polypropylenes, polymethylmethacrylate, polyvinyl chloride, polyesters, polyethers, and polyethylene. A polymer core spherical nucleic acid comprises a polymer core, and a shell of oligonucleotides attached to the external side of the polymer core.

Biodegradable, biopolymer (e.g., polypeptides such as BSA, polysaccharides, etc.), other biological materials (e.g., carbohydrates), and/or polymeric compounds are also contemplated for use in producing nanoparticles. In some embodiments, the polymer is polylactide, a polylactide-polyglycolide copolymer, a polycaprolactone, a polyacrylate, alginate, albumin, silica, polypyrrole, polythiophene, polyaniline, polyethylenimine, poly(methyl methacrylate), chitosan, or a related structure. In some embodiments, the polymer is poly(lactic-co-glycolic acid) (PLGA).

### Protein Core Spherical Nucleic Acids (ProSNAs)

In any of the aspects or embodiments of the disclosure, the nanoparticle core is a protein core. A protein core spherical nucleic acid (ProSNA) comprises a protein core, and a shell of oligonucleotides attached to the external side of the protein core. As used herein, protein is used interchangeably with “polypeptide” and refers to one or more polymers of amino acid residues. In various embodiments of the disclosure, a protein core comprises or consists of a single protein (i.e., a single polymer of amino acids), a multimeric protein, a peptide (e.g., a polymer of amino acids that between about 2 and 50 amino acids in length), or a synthetic fusion protein of two or more proteins. Synthetic fusion proteins include, without limitation, an expressed fusion protein (expressed from a single gene) and post-expression fusions where proteins are conjugated together chemically. Protein/oligonucleotide core-shell nanoparticles are also generally described in U.S. Patent Application Publication No. 2017/0232109, which is incorporated by reference herein in its entirety.

In further embodiments, the protein core is an enzyme, a therapeutic protein, a structural protein, a defensive protein, a storage protein, a transport protein, a hormone, a receptor protein, a motor protein, an immunogenic protein, or a fluorescent protein. In some embodiments, the protein core is a structural protein, a storage protein, or a transport protein. In some embodiments, an oligonucleotide in the shell of oligonucleotides is attached to the protein core are attached to a lysine or cysteine residue of the protein core. In some embodiments, the one or more double stranded oligonucleotides in the shell of oligonucleotides are attached to a lysine or cysteine residue of the protein core.

Methods of attaching oligonucleotides to a protein core are described, e.g., in U.S. Patent Application Publication No. 2017/0232109 and Brodin et al., J Am Chem Soc. 137(47): 14838-41 (2015), each of which is incorporated by reference herein in its entirety. In general, an oligonucleotide can be modified at a terminus with an alkyne group, e.g., a DBCO-type group for reaction with the azide of the protein surface:

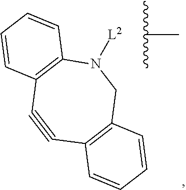

where L is a linker to a terminus of the oligonucleotide. L² can be C₁₋₁₀ alkylene, —C(O)—C₁₋₁₀ alkylene-Y—, and —C(O)—C₁₋₁₀ alkylene-Y—C₁₋₁₀ alkylene-(OCH₂CH₂)ₘ—Y—; wherein each Y is independently selected from the group consisting of a bond, C(O), O, NH, C(O)NH, and NHC(O); and m is 0, 1, 2, 3, 4, or 5. For example, the DBCO functional group can be attached via a linker having a structure of

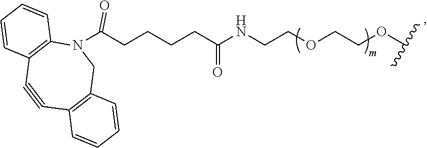

where the terminal “O” is from a terminal nucleotide on the oligonucleotide. Use of this DBCO-type moiety results in a structure between the oligonucleotide and the protein, in cases where a surface amine is modified, of:

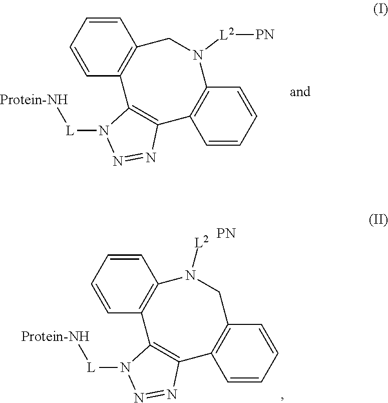

where L and L² are each independently selected from C₁₋₁₀ alkylene, —C(O)—C₁₋₁₀ alkylene-Y—, and —C(O)—C₁₋₁₀ alkylene-Y—C₁₋₁₀ alkylene-(OCH₂CH₂)ₘY—; each Y is independently selected from the group consisting of a bond, C(O), O, NH, C(O)NH, and NHC(O); m is 0, 1, 2, 3, 4, or 5; and PN is the oligonucleotide. Similar structures where a surface thiol or surface carboxylate of the protein are modified can be made in a similar fashion to result in comparable linkage structures.

The protein can be modified at a surface functional group (e.g., a surface amine, a surface carboxylate, a surface thiol) with a linker that terminates with an azide functional group: Protein-X-L-N₃, X is from a surface amino group (e.g., —NH—), carboxylic group (e.g., —C(O)— or —C(O)O—), or thiol group (e.g., —S—) on the protein; L is selected from C₁₋₁₀ alkylene, —Y—C(O)—C₁₋₁₀ alkylene-Y—, and —Y—C(O)—C₁₋₁₀ alkylene-Y—C₁₋₁₀ alkylene-(OCH₂CH₂)ₘ—Y—; each Y is independently selected from the group consisting of a bond, C(O), O, NH, C(O)NH, and NHC(O); and m is 0, 1, 2, 3, 4, or 5. Introduction of the “L-N₃” functional group to the surface group of the protein can be accomplished using well-known techniques. For example, a surface amine of the protein can be reacted with an activated ester of a linker having a terminal N₃ to form an amide bond between the amine of the protein and the carboxylate of the activated ester of the linker reagent.

The oligonucleotide can be modified to include an alkyne functional group at a terminus of the oligonucleotide: Oligonucleotide-L₂-X-≡—R; L² is selected from C₁₋₁₀ alkylene, —C(O)—C₁₋₁₀ alkylene-Y—, and —C(O)—C₁₋₁₀ alkylene-Y—C₁₋₁₀ alkylene-(OCH₂CH₂)ₘ—Y—; each Y is independently selected from the group consisting of a bond, C(O), O, NH, C(O)NH, and NHC(O); m is 0, 1, 2, 3, 4, or 5; and X is a bond and R is H or C₁₋₁₀alkyl; or X and R together with the carbons to which they are attached form a 8-10 membered carbocyclic or 8-10 membered heterocyclic group. In some cases, the oligonucleotide has a structure

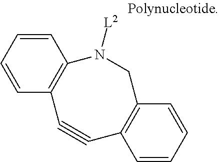

The protein, with the surface modified azide, and the oligonucleotide, with a terminus modified to include an alkyne, can be reacted together to form a triazole ring in the presence of a copper (II) salt and a reducing agent to generate a copper (I) salt in situ. In some cases, a copper (I) salt is directly added. Contemplated reducing agents include ascorbic acid, an ascorbate salt, sodium borohydride, 2-mercaptoethanol, dithiothreitol (DTT), hydrazine, lithium aluminum hydride, diisobutylaluminum hydride, oxalic acid, Lindlar catalyst, a sulfite compound, a stannous compound, a ferrous compound, sodium amalgam, tris(2-carboxyethyl)phosphine, hydroquinone, and mixtures thereof.

The surface functional group of the protein can be attached to the oligonucleotide using other attachment chemistries. For example, a surface amine can be directed conjugated to a carboxylate or activated ester at a terminus of the oligonucleotide, to form an amide bond. A surface carboxylate can be conjugated to an amine on a terminus of the oligonucleotide to form an amide bond. Alternatively, the surface carboxylate can be reacted with a diamine to form an amide bond at the surface carboxylate and an amine at the other terminus. This terminal amine can then be modified in a manner similar to that for a surface amine of the protein. A surface thiol can be conjugated with a thiol moiety on the oligonucleotide to form a disulfide bond. Alternatively, the thiol can be conjugated with an activated ester on a terminus of a oligonucleotide to form a thiocarboxylate.

In some aspects, the disclosure provides a ProSNA comprising or consisting of (a) a protein core; (b) a shell of oligonucleotides comprising one or more oligonucleotides attached to the protein core; and (c) a hybrid peptide. In some embodiments, one or more oligonucleotides in the shell of oligonucleotides is modified on its 5′ end and/or 3′ end with dibenzocyclooctyl (DBCO). In various embodiments, the shell of oligonucleotides comprises single-stranded DNA, double-stranded DNA, single-stranded RNA, double-stranded RNA, or a combination thereof. In some embodiments, each oligonucleotide in the shell of oligonucleotides is covalently attached to the protein core. In some embodiments, each oligonucleotide in the shell of oligonucleotides is attached to the protein core through a linker. In various embodiments, the linker is a cleavable linker or a non-cleavable linker. In some embodiments, the linker is SPDP succinimidyl 3-(2-pyridyldithio)propionate. In some embodiments, at least one oligonucleotide in the shell of oligonucleotides is attached to the protein core in the following configuration: protein core-—NH—C(O)—(CH₂)₅—NH—C(O)—C₂—S—S-oligonucleotide-NH-PEG₃-Folate. In some embodiments, each oligonucleotide in the shell of oligonucleotides is attached to the protein core in the following configuration: protein core--NH—C(O)—(CH₂)₅—NH—C(O)—C₂—S—S-oligonucleotide-NH-PEG₃-Folate. In some embodiments, the linker is NHS-PEG(x)-Azide, wherein X is 3 or 4. In some embodiments, at least one oligonucleotide in the shell of oligonucleotides is attached to the protein core in the following configuration: protein core--NH—(CO)-PEG_{X}-Triazole-oligonucleotide-NH—C(O)-PEG₃-Folate. In some embodiments, each oligonucleotide in the shell of oligonucleotides is attached to the protein core in the following configuration: protein core-—NH-(CO)-PEG_{X}-Triazole-oligonucleotide-NH—C(O)-PEG₃-Folate. In some embodiments, one or more or all oligonucleotides in the shell of oligonucleotides is non-covalently attached to the protein core.

### Inorganic Core Spherical Nucleic Acids

The disclosure contemplates nanoparticle cores that comprise a variety of inorganic materials including, but not limited to, metals, semi-conductor materials or ceramics as described in U.S. Patent Publication No 20030147966. For example, metal-based nanoparticles include those described herein. Ceramic nanoparticle materials include, but are not limited to, brushite, tricalcium phosphate, alumina, silica, and zirconia. An inorganic core spherical nucleic acid comprises an inorganic core, and a shell of oligonucleotides attached to the external side of the inorganic core.

In some embodiments, the nanoparticle is metallic, and in various aspects, the nanoparticle is a colloidal metal. Thus, in various embodiments, nanoparticles useful in the practice of the methods include metal (including for example and without limitation, gold, silver, platinum, aluminum, palladium, copper, cobalt, indium, nickel, or any other metal amenable to nanoparticle formation), semiconductor (including for example and without limitation, CdSe, CdS, and CdS or CdSe coated with ZnS) and magnetic (for example, ferromagnetite) colloidal materials. Other nanoparticles useful in the practice of the technology disclosed herein include, also without limitation, ZnS, ZnO, Ti, TiO₂, Sn, SnO₂, Si, SiO₂, Fe, Fe⁺⁴, Ag, Cu, Ni, Al, steel, cobalt-chrome alloys, Cd, titanium alloys, AgI, AgBr, HgI₂, PbS, PbSe, ZnTe, CdTe, In₂S₃, In₂Se₃, Cd₃P₂, Cd₃As₂, InAs, and GaAs. Methods of making ZnS, ZnO, TiO₂, AgI, AgBr, HgI₂, PbS, PbSe, ZnTe, CdTe, In₂S₃, In₂Se₃, Cd₃P₂, Cd₃As₂, InAs, and GaAs nanoparticles are also known in the art. See, e.g., Weller, Angew. Chem. Int. Ed. Engl., 32, 41 (1993); Henglein, Top. Curr. Chem., 143, 113 (1988); Henglein, Chem. Rev., 89, 1861 (1989); Brus, Appl. Phys. A., 53, 465 (1991); Bahncmann, in Photochemical Conversion and Storage of Solar Energy (eds. Pelizetti and Schiavello 1991), page 251; Wang and Herron, J. Phys. Chem., 95, 525 (1991); Olshavsky, et al., J. Am. Chem. Soc., 112, 9438 (1990); Ushida et al., J. Phys. Chem., 95, 5382 (1992). In some embodiments, the nanoparticle is an iron oxide nanoparticle. In further embodiments, the nanoparticle core is gold, silver, platinum, aluminum, palladium, copper, cobalt, indium, cadmium selenide, iron oxide, fullerene, metal-organic framework, zinc sulfide, or nickel.

Methods of making metal, semiconductor and magnetic nanoparticles are well-known in the art. See, for example, Schmid, G. (ed.) Clusters and Colloids (VCH, Weinheim, 1994); Hayat, M. A. (ed.) Colloidal Gold: Principles, Methods, and Applications (Academic Press, San Diego, 1991); Massart, R., IEEE Transactions On Magnetics, 17, 1247 (1981); Ahmadi, T. S. et al., Science, 272, 1924 (1996); Henglein, A. et al., J. Phys. Chem., 99, 14129 (1995); Curtis, A. C., et al., Angew. Chem. Int. Ed. Engl., 27, 1530 (1988). Preparation of polyalkylcyanoacrylate nanoparticles prepared is described in Fattal, et al., J. Controlled Release (1998) 53: 137-143 and U.S. Pat. No. 4,489,055. Methods for making nanoparticles comprising poly(D-glucaramidoamine)s are described in Liu, et al., J. Am. Chem. Soc. (2004) 126:7422-7423. Preparation of nanoparticles comprising polymerized methylmethacrylate (MMA) is described in Tondelli, et al., Nucl. Acids Res. (1998) 26:5425-5431, and preparation of dendrimer nanoparticles is described in, for example Kukowska-Latallo, et al., Proc. Natl. Acad. Sci. USA (1996) 93:4897-4902 (Starburst polyamidoamine dendrimers).

### Oligonucleotides

The disclosure provides spherical nucleic acids (e.g., LSNAs, LNP-SNAs, polymer SNAs, ProSNAs, and inorganic SNAs) comprising a nanoparticle core, and a shell of oligonucleotides comprising one or more oligonucleotides attached to the nanoparticle core, and a hybrid peptide attached to one or more oligonucleotides in the shell of oligonucleotides. Oligonucleotides contemplated for use according to the disclosure include those attached to a nanoparticle core through any means (e.g., covalent or non-covalent attachment). Oligonucleotides of the disclosure include, in various embodiments, DNA oligonucleotides, RNA oligonucleotides, modified forms thereof, or a combination thereof. In any aspects or embodiments described herein, an oligonucleotide is single-stranded, double-stranded, or partially double-stranded. In any aspects or embodiments of the disclosure, an oligonucleotide comprises a detectable marker.

As described herein, modified forms of oligonucleotides are also contemplated by the disclosure which include those having at least one modified internucleotide linkage. In some embodiments, the oligonucleotide is all or in part a peptide nucleic acid. Other modified internucleoside linkages include at least one phosphorothioate linkage. Still other modified oligonucleotides include those comprising one or more universal bases. “Universal base” refers to molecules capable of substituting for binding to any one of A, C, G, T and U in nucleic acids by forming hydrogen bonds without significant structure destabilization. The oligonucleotide incorporated with the universal base analogues is able to function, e.g., as a probe in hybridization. Examples of universal bases include but are not limited to 5′-nitroindole-2′-deoxyriboside, 3-nitropyrrole, inosine and hypoxanthine.

The term “nucleotide” or its plural as used herein is interchangeable with modified forms as discussed herein and otherwise known in the art. The term “nucleobase” or its plural as used herein is interchangeable with modified forms as discussed herein and otherwise known in the art. Nucleotides or nucleobases comprise the naturally occurring nucleobases A, G, C, T, and U. Non-naturally occurring nucleobases include, for example and without limitations, xanthine, diaminopurine, 8-oxo-N6-methyladenine, 7-deazaxanthine, 7-deazaguanine, N4,N4-ethanocytosin, N′,N′-ethano-2,6-diaminopurine, 5-methylcytosine (mC), 5-(C3-C6)-alkynyl-cytosine, 5-fluorouracil, 5-bromouracil, pseudoisocytosine, 2-hydroxy-5-methyl-4-tr-iazolopyridin, isocytosine, isoguanine, inosine and the “non-naturally occurring” nucleobases described in Benner et al., U.S. Pat. No. 5,432,272 and Susan M. Freier and Karl-Heinz Altmann, 1997, Nucleic Acids Research, vol. 25: pp 4429-4443. The term “nucleobase” also includes not only the known purine and pyrimidine heterocycles, but also heterocyclic analogues and tautomers thereof. Further naturally and non-naturally occurring nucleobases include those disclosed in U.S. Pat. No. 3,687,808 (Merigan, et al.), in Chapter 15 by Sanghvi, in Antisense Research and Application, Ed. S. T. Crooke and B. Lebleu, CRC Press, 1993, in Englisch et al., 1991, Angewandte Chemie, International Edition, 30: 613-722 (see especially pages 622 and 623, and in the Concise Encyclopedia of Polymer Science and Engineering, J. I. Kroschwitz Ed., John Wiley & Sons, 1990, pages 858-859, Cook, Anti-Cancer Drug Design 1991, 6, 585-607, each of which are hereby incorporated by reference in their entirety). In various aspects, oligonucleotides also include one or more “nucleosidic bases” or “base units” which are a category of non-naturally-occurring nucleotides that include compounds such as heterocyclic compounds that can serve like nucleobases, including certain “universal bases” that are not nucleosidic bases in the most classical sense but serve as nucleosidic bases. Universal bases include 3-nitropyrrole, optionally substituted indoles (e.g., 5-nitroindole), and optionally substituted hypoxanthine. Other desirable universal bases include, pyrrole, diazole or triazole derivatives, including those universal bases known in the art.

Examples of oligonucleotides include those containing modified backbones or non-natural internucleoside linkages. Oligonucleotides having modified backbones include those that retain a phosphorus atom in the backbone and those that do not have a phosphorus atom in the backbone. Modified oligonucleotides that do not have a phosphorus atom in their internucleoside backbone are considered to be within the meaning of “oligonucleotide”.

Modified oligonucleotide backbones containing a phosphorus atom include, for example, phosphorothioates, chiral phosphorothioates, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, selenophosphates and boranophosphates having normal 3′-5′ linkages, 2′-5′ linked analogs of these, and those having inverted polarity wherein one or more internucleotide linkages is a 3′ to 3′, 5′ to 5′ or 2′ to 2′ linkage. Also contemplated are oligonucleotides having inverted polarity comprising a single 3′ to 3′ linkage at the 3′-most internucleotide linkage, i.e. a single inverted nucleoside residue which may be abasic (the nucleotide is missing or has a hydroxyl group in place thereof). Salts, mixed salts and free acid forms are also contemplated. Representative United States patents that teach the preparation of the above phosphorus-containing linkages include, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; 5,194,599; 5,565,555; 5,527,899; 5,721,218; 5,672,697 and 5,625,050, the disclosures of which are incorporated by reference herein.

Modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages; siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts. See, for example, U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; 5,792,608; 5,646,269 and 5,677,439, the disclosures of which are incorporated herein by reference in their entireties.

In still further embodiments, oligonucleotide mimetics wherein both one or more sugar and/or one or more internucleotide linkage of the nucleotide units are replaced with “non-naturally occurring” groups. The bases of the oligonucleotide are maintained for hybridization. In some aspects, this embodiment contemplates a peptide nucleic acid (PNA).

In PNA compounds, the sugar-backbone of an oligonucleotide is replaced with an amide containing backbone. See, for example U.S. Pat. Nos. 5,539,082; 5,714,331; and 5,719,262; see also Nielsen et al., Science, 1991, 254, 1497-1500, the disclosures of which are herein incorporated by reference.

In still further embodiments, oligonucleotides are provided with phosphorothioate backbones and oligonucleosides with heteroatom backbones, and including —CH2-NH—O—CH2-, —CH2-N(CH3)-O—CH2-, —CH2-O—N(CH3)-CH2-, —CH2-N(CH3)-N(CH3)-CH2- and —O—N(CH3)-CH2-CH2- described in U.S. Pat. Nos. 5,489,677, and 5,602,240. Also contemplated are oligonucleotides with morpholino backbone structures described in U.S. Pat. No. 5,034,506.

In various forms, the linkage between two successive monomers in the oligonucleotide consists of 2 to 4, desirably 3, groups/atoms selected from —CH₂—, —O—, —S—, —NR^{H}—, >C═O, >C═NR^{H}, >C═S, —Si(R″)₂—, —SO—, —S(O)₂—, —P(O)₂—, —PO(BH₃)—, —P(O,S)—, —P(S)₂—, —PO(R″)—, —PO(OCH₃)—, and —PO(NHRH)_, where R^{H} is selected from hydrogen and C₁₋₄-alkyl, and R″ is selected from C₁₋₆-alkyl and phenyl. Illustrative examples of such linkages are —CH₂—CH₂—CH₂—, —CH₂—CO—CH₂—, —CH₂—CHOH—CH₂—, —O—CH₂—O—, —O—CH₂—CH₂—, —OCH₂—CH═ (including R⁵ when used as a linkage to a succeeding monomer), —CH₂—CH₂—O—, —NR^{H}—CH₂—CH₂—, —CH₂—CH₂—NR^{H}—, —CH₂—NR^{H}—CH₂—, —O—CH₂—CH₂—NR^{H}—, —NR^{H}—CO—O—, —NR^{H}—CO—NR^{H}—, —NR^{H}—CS—NR^{H}—, —NR^{H}—C(═NR^{H})—NR^{H}—, —NR^{H}—CO—CH₂—NR^{H}—O—CO—O—, —O—CO—CH₂—O—, —O—CH₂—CO—O—, —CH₂—CO—NR^{H}—, —O—CO—NR^{H}—, —NR^{H}—CO—CH₂—, —O—CH₂—CO—NR^{H}—, —O—CH₂—CH₂—NR^{H}—, —CH═N—O—, —CH₂—NR^{H}—O—, —CH₂—O—N═ (including R⁵ when used as a linkage to a succeeding monomer), —CH₂—O—NR^{H}—, —CO—NR^{H}—CH₂—, —CH₂—NR^{H}—O—, —CH₂—NR^{H}—CO—, —O—NR^{H}—CH₂—, —O—NR^{H}, —O—CH₂—S—, —S—CH₂—O—, —CH₂—CH₂—S—, —O—CH₂—CH₂—S—, —S—CH₂—CH═ (including R⁵ when used as a linkage to a succeeding monomer), —S—CH₂—CH₂—, —S—CH₂—CH₂—O—, —S—CH₂—CH₂—S—, —CH₂—S—CH₂—, —CH₂—SO—CH₂—, —CH₂—SO₂—CH₂—, —O—SO—O—, —O—S(O)₂—O—, —O—S(O)₂—CH₂—, —O—S(O)₂—NR^{H}—, —NR^{H}—S(O)₂—CH₂—; —O—S(O)₂—CH₂—, —O—P(O)₂—O—, —O—P(O,S)—O—, —O—P(S)₂—O—, —S—P(O)₂—O—, —S—P(O,S)—O—, —S—P(S)₂—O—, —O—P(O)₂—S—, —O—P(O,S)—S—, —O—P(S)₂—S—, —S—P(O)₂—S—, —S—P(O,S)—S—, —S—P(S)₂—S—, —O—PO(R″)—O—, —O—PO(OCH₃)—O—, —O—PO(O CH₂CH₃)—O—, —O—PO(O CH₂CH₂S—R)—O—, —O—PO(BH₃)—O—, —O—PO(NHR^{N})—O—, —O—P(O)₂—NR^{H} H—, —NR^{H}—P(O)₂—O—, —O—P(O,NR^{H})—, —CH₂—P(O)₂—O—, —O—P(O)₂—CH₂—, and —O—Si(R″)₂—O—; among which —CH₂—CO—NR^{H}—, —CH₂—NR^{H}—O—, —S—CH₂—O—, —O—P(O)₂—O—O—P(—O,S)—O—, —O—P(S)₂—O—, —NR^{H} P(O)₂—O—, —O—P(O,NR^{H})—O—O—PO(R″)—O—O—PO(CH₃)—O—, and —O—PO(NHR^{N})—O—, where R^{H} is selected form hydrogen and C₁₋₄-alkyl, and R″ is selected from C₁₋₆-alkyl and phenyl, are contemplated. Further illustrative examples are given in Mesmaeker Et. al., Current Opinion in Structural Biology 1995, 5, 343-355 and Susan M. Freier and Karl-Heinz Altmann, Nucleic Acids Research, 1997, vol 25, pp 4429-4443.

Still other modified forms of oligonucleotides are described in detail in U.S. patent application No. 20040219565, the disclosure of which is incorporated by reference herein in its entirety.

Modified oligonucleotides may also contain one or more substituted sugar moieties. In certain aspects, oligonucleotides comprise one of the following at the 2′ position: OH; F; O—, S—, or N-alkyl; O—, S—, or N-alkenyl; O—, S- or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. Other embodiments include O[(CH₂)ₙO]ₘCH₃, O(CH₂)ₙOCH₃, O(CH₂)ₙNH₂, O(CH₂)ₙCH₃, O(CH₂)ₙONH₂, and O(CH₂)ₙON[(CH₂)ₙCH₃]₂, where n and m are from 1 to about 10. Other oligonucleotides comprise one of the following at the 2′ position: C₁ to C₁₀ lower alkyl, substituted lower alkyl, alkenyl, alkynyl, alkaryl, aralkyl, O-alkaryl or O-aralkyl, SH, SCH₃, OCN, C, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, or an RNA cleaving group. In one aspect, a modification includes 2′-methoxyethoxy (2′-O—CH₂CH₂OCH₃, also known as 2′-O—(2-methoxyethyl) or 2′-MOE) (Martin et al., Helv. Chim. Acta, 1995, 78, 486-504) i.e., an alkoxyalkoxy group. Other modifications include 2′-dimethylaminooxyethoxy, i.e., a O(CH₂)20N(CH₃)₂ group, also known as 2′-DMAOE, and 2′-dimethylaminoethoxyethoxy (also known in the art as 2′-O-dimethyl-amino-ethoxy-ethyl or 2′-DMAEOE), i.e., 2′-O—CH₂—O—CH₂—N(CH₃)₂.

Still other modifications include 2′-methoxy (2′-O—CH₃), 2′-aminopropoxy (2′-OCH₂CH₂CH₂NH₂), 2′-allyl (2′-CH₂—CH═CH₂), 2′-O-allyl (2′-O—CH₂—CH═CH₂) and 2′-fluoro (2′-F). The 2′-modification may be in the arabino (up) position or ribo (down) position. In one aspect, a 2′-arabino modification is 2′-F. Similar modifications may also be made at other positions on the oligonucleotide, for example, at the 3′ position of the sugar on the 3′ terminal nucleotide or in 2′-5′ linked oligonucleotides and the 5′ position of 5′ terminal nucleotide. Oligonucleotides may also have sugar mimetics such as cyclobutyl moieties in place of the pentofuranosyl sugar. See, for example, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 5,792,747; and 5,700,920, the disclosures of which are incorporated by reference in their entireties herein.

In some aspects, a modification of the sugar includes Locked Nucleic Acids (LNAs) in which the 2′-hydroxyl group is linked to the 3′ or 4′ carbon atom of the sugar ring, thereby forming a bicyclic sugar moiety. The linkage is in certain aspects is a methylene (—CH₂—)n group bridging the 2′ oxygen atom and the 4′ carbon atom wherein n is 1 or 2. LNAs and preparation thereof are described in WO 98/39352 and WO 99/14226.

Modified nucleotides are described in EP 1 072 679 and WO 97/12896, the disclosures of which are incorporated herein by reference. Modified nucleobases include without limitation, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Further modified bases include tricyclic pyrimidines such as phenoxazine cytidine(1H-pyrimido[5,4-b][1,4]benzoxazin-2(3H)-one), phenothiazine cytidine (1H-pyrimido[5,4-b][1,4]benzothiazin-2(3H)-one), G-clamps such as a substituted phenoxazine cytidine (e.g. 9-(2-aminoethoxy)-H-pyrimido[5,4-b][1,4]benzox-azin-2(3H)-one), carbazole cytidine (2H-pyrimido[4,5-b]indol-2-one), pyridoindole cytidine (H-pyrido[3′,2′:4,5]pyrrolo[2,3-d]pyrimidin-2-one). Modified bases may also include those in which the purine or pyrimidine base is replaced with other heterocycles, for example 7-deaza-adenine, 7-deazaguanosine, 2-aminopyridine and 2-pyridone. Additional nucleobases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., 1991, Angewandte Chemie, International Edition, 30: 613, and those disclosed by Sanghvi, Y. S., Chapter 15, Antisense Research and Applications, pages 289-302, Crooke, S. T. and Lebleu, B., ed., CRC Press, 1993. Certain of these bases are useful for increasing binding affinity and include 5-substituted pyrimidines, 6-azapyrimidines and N-2, N-6 and 0-6 substituted purines, including 2-aminopropyladenine, 5-propynyluracil and 5-propynylcytosine. 5-methylcytosine substitutions have been shown to increase nucleic acid duplex stability by 0.6-1.2° C. and are, in certain aspects combined with 2′-O-methoxyethyl sugar modifications. See, U.S. Pat. Nos. 3,687,808, 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,645,985; 5,830,653; 5,763,588; 6,005,096; 5,750,692 and 5,681,941, the disclosures of which are incorporated herein by reference.

Methods of making polynucleotides of a predetermined sequence are well-known. See, e.g., Sambrook et al., Molecular Cloning: A Laboratory Manua^{l} (2nd ed. 1989) and F. Eckstein (ed.) Oligonucleotides and Analogue^{s}, 1st Ed. (Oxford University Press, New York, 1991). Solid-phase synthesis methods are preferred for both polyribonucleotides and polydeoxyribonucleotides (the well-known methods of synthesizing DNA are also useful for synthesizing RNA). Polyribonucleotides can also be prepared enzymatically. Non-naturally occurring nucleobases can be incorporated into the polynucleotide, as well. See, e.g., U.S. Pat. No. 7,223,833; Katz, J. Am. Chem. Soc., 74:2238 (1951); Yamane, et al., J. Am. Chem. Soc., 83:2599 (1961); Kosturko, et al., Biochemistry, 13:3949 (1974); Thomas, J. Am. Chem. Soc., 76:6032 (1954); Zhang, et al., J. Am. Chem. Soc., 127:74-75 (2005); and Zimmermann, et al., J. Am. Chem. Soc., 124:13684-13685 (2002).

In various aspects, an oligonucleotide of the disclosure, or a modified form thereof, is generally about 5 nucleotides to about 100 nucleotides in length. More specifically, an oligonucleotide of the disclosure is about 5 to about 100, about 5 to about 90 nucleotides in length, about 5 to about 80 nucleotides in length, about 5 to about 70 nucleotides in length, about 5 to about 60 nucleotides in length, about 5 to about 50 nucleotides in length about 5 to about 45 nucleotides in length, about 5 to about 40 nucleotides in length, about 5 to about 35 nucleotides in length, about 5 to about 30 nucleotides in length, about 5 to about 25 nucleotides in length, about 5 to about 20 nucleotides in length, about 5 to about 15 nucleotides in length, about 5 to about 10 nucleotides in length, about 10 to about 100 nucleotides in length, about 10 to about 90 nucleotides in length, about 10 to about 80 nucleotides in length, about 10 to about 70 nucleotides in length, about 10 to about 60 nucleotides in length, about 10 to about 50 nucleotides in length about 10 to about 45 nucleotides in length, about 10 to about 40 nucleotides in length, about 10 to about 35 nucleotides in length, about 10 to about 30 nucleotides in length, about 10 to about 25 nucleotides in length, about 10 to about 20 nucleotides in length, about 10 to about 15 nucleotides in length, about 18 to about 28 nucleotides in length, about 15 to about 26 nucleotides in length, and all oligonucleotides intermediate in length of the sizes specifically disclosed to the extent that the oligonucleotide is able to achieve the desired result. Accordingly, in various embodiments, an oligonucleotide of the disclosure is or is at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more nucleotides in length. In further embodiments, an oligonucleotide of the disclosure is less than 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more nucleotides in length. In various embodiments, the shell of oligonucleotides attached to the exterior of the nanoparticle core of the SNA comprises a plurality of oligonucleotides that all have the same length/sequence, while in some embodiments, the plurality of oligonucleotides comprises one or more oligonucleotide that have a different length and/or sequence relative to at least one other oligonucleotide in the plurality. For example, and without limitation, in some embodiments the shell of oligonucleotides comprises a plurality of immunostimulatory oligonucleotides, wherein one immunostimulatory oligonucleotide has a sequence that is different than at least one other immunostimulatory oligonucleotide in the plurality.

In some embodiments, one or more oligonucleotides in the shell of oligonucleotides comprises a CpG 1826 oligonucleotide (SEQ ID NO: 1). In various embodiments, one or more oligonucleotides in the shell of oligonucleotides comprises a CpG 1018 oligonucleotide (SEQ ID NO: 2), a CpG 2006 oligonucleotide (SEQ ID NO: 3), or a CpG 2395 oligonucleotide (SEQ ID NO: 11).

In some embodiments, an oligonucleotide in the shell of oligonucleotides is a targeting oligonucleotide, such as an aptamer. Accordingly, all features and aspects of oligonucleotides described herein (e.g., length, type (DNA, RNA, modified forms thereof), optional presence of spacer) also apply to aptamers. Aptamers are oligonucleotide sequences that can be evolved to bind to various target analytes of interest. Aptamers may be single stranded, double stranded, or partially double stranded.

### CpG Motifs and Toll-Like Receptors

Toll-like receptors (TLRs) are a class of proteins, expressed in sentinel cells, that play a key role in regulation of innate immune system. The mammalian immune system uses two general strategies to combat infectious diseases. Pathogen exposure rapidly triggers an innate immune response that is characterized by the production of immunostimulatory cytokines, chemokines and polyreactive IgM antibodies. The innate immune system is activated by exposure to Pathogen Associated Molecular Patterns (PAMPs) that are expressed by a diverse group of infectious microorganisms. The recognition of PAMPs is mediated by members of the Toll-like family of receptors. CpG motifs, cytosine-guanine dinucleotide sequences, are exemplary PAMPs recognized by TLR receptors. TLR receptors, such as TLR 8 and TLR 9 that respond to specific oligonucleotides are located inside special intracellular compartments, called endosomes. The mechanism of modulation of, for example and without limitation, TLR 8 and TLR 9 receptors, is based on DNA-protein interactions.

Accordingly, in some embodiments, methods of utilizing SNAs as described herein for modulating toll-like receptors are disclosed. The method up-regulates the Toll-like-receptor activity through the use of a TLR agonist. The method comprises contacting a cell having a toll-like receptor with a SNA of the disclosure, thereby modulating the activity and/or the expression of the toll-like receptor. The toll-like receptors modulated include one or more of toll-like receptor 1, toll-like receptor 2, toll-like receptor 3, toll-like receptor 4, toll-like receptor 5, toll-like receptor 6, toll-like receptor 7, toll-like receptor 8, toll-like receptor 9, toll-like receptor 10, toll-like receptor 11, toll-like receptor 12, and/or toll-like receptor 13.

### Compositions, Pharmaceutical Formulations, and Antigenic Compositions

The disclosure also provides compositions that comprise a hybrid peptide of the disclosure, an SNA of the disclosure, or a combination thereof. In some embodiments, the composition comprising a hybrid peptide comprises one or more antigens attached to a Cathepsin S-sensitive linker. In some embodiments, the composition comprising a hybrid peptide comprises an antigen attached to one or more Cathepsin S-sensitive linkers. In some embodiments, the composition comprising a hybrid peptide comprises one or more antigens attached to one or more Cathepsin S-sensitive linkers. In some embodiments, the composition comprises an SNA comprising any one of the hybrid peptides described above. In some embodiments, the composition comprises a plurality of the SNAs or hybrid peptides described above. In some embodiments, at least two of the plurality comprise a different antigen, relative to each other.

In some embodiments, the composition is an antigenic composition. In some embodiments, the composition further comprises a pharmaceutically acceptable carrier. The term “carrier” refers to a vehicle within which the hybrid peptide or the SNA as described herein is administered to a subject. Any conventional media or agent that is compatible with the SNAs according to the disclosure can be used. The term carrier encompasses diluents, excipients, adjuvants and a combination thereof. Pharmaceutically acceptable carriers are well known in the art (see, e.g., Remington's Pharmaceutical Sciences by Martin, 1975, the entire disclosure of which is herein incorporated by reference).

Exemplary “diluents” include sterile liquids such as sterile water, saline solutions, and buffers (e.g., phosphate, tris, borate, succinate, or histidine), and further include water for injection, saline solution, buffers such as Tris, acetates, citrates or phosphates, fixed oils, polyethylene glycols, glycerine, propylene glycol or other synthetic solvents. Exemplary “excipients” are inert substances that may enhance vaccine stability and include but are not limited to polymers (e.g., polyethylene glycol), carbohydrates (e.g., starch, glucose, lactose, sucrose, or cellulose), alcohols (e.g., glycerol, sorbitol, or xylitol), and pH-adjusting agents.

### Methods of Inducing an Immune Response

The disclosure also includes methods of treating, reducing the symptoms of, or ameliorating a disease in a subject comprising administering to the subject an effective amount of a hybrid peptide or SNA, or a combination thereof, of the disclosure (e.g., administered as a composition, pharmaceutical formulation, or antigenic composition), thereby treating the disease in the subject. Diseases or disorders that are contemplated by the disclosure in such methods include, but are not limited to, cancer.

Naïve CD8⁺ T cells are continuously circulating through the body, migrating through the blood and secondary lymphoid tissues, such as lymph nodes and the spleen (Weninger et al., J Exp Med, 194: 953-66, 2001). Once a naïve T cell encounters and recognizes its cognate antigen presented on an MHC class I complex on an APC, the CD8⁺ T cell undergoes a number of changes leading to its activation (Huster et al., PNAS, 101: 5610-15, 2004). Activated CD8⁺ T cells rapidly expand, and migrate from the secondary lymphoid tissue to a tissue displaying its cognate antigen, such as a tumor-specific antigen on the surface of a tumor cell (Joshi et al., Immunity, 27: 281-95, 2007). The activated CD8⁺ T cell recognizes a cell displaying the cognate antigen, and kills that cell using perforin and granzyme B. Upon elimination of cells expressing the cognate antigen, the effector population contracts through activation induced cell death (AICD), leaving only a small percentage alive that are termed memory T cells (Lau et al., Nature, 369: 648-52, 1994). Memory CD8⁺ T cells are a long-lived antigen-specific population. A subset of memory T cells, effector memory T cells, have a phenotype of CD44⁺ and CD62L⁻. Effector memory T cells circulate throughout the body, and rapidly expand if the same cognate antigen is encountered again (Mackay, J Exp Med, 171: 801-17, 1990; Sallusto et al., Nature, 401: 708-12, 1999).

The disclosure also includes methods for eliciting an immune response in a subject in need thereof, comprising administering to the subject an effective amount of a hybrid peptide, an SNA, or a combination thereof (e.g., formulated as an antigenic composition) of the disclosure. As exemplified herein, moieties and SNAs of the disclosure induce an antigen-specific immune response. In various embodiments, administering SNAs of the disclosure (e.g., formulated as a composition, pharmaceutical formulation, or antigenic composition) to a subject results in the induction of an antigen-specific CD8⁺ T cell response. In other embodiments, administering the SNAs of the disclosure to a subject results in the induction of an antigen-specific CD8⁺ T cell response, and subsequent generation of CD44⁺/CD62L⁻ effector memory T cells and/or KLRG1⁺/CD127⁻ short-lived effector T cells.

The immune response raised by the methods of the present disclosure generally includes a CD8⁺ T cell-mediated response. Administration of the SNA comprising a hybrid peptide alone, administration of the hybrid peptide alone, and administration of a composition comprising the SNA comprising a hybrid peptide and a hybrid peptide separate from the SNA, as disclosed herein, generates an immune response that recognizes and selectively targets cancer as described herein. Methods for assessing T cell responses after administration of an antigenic composition (immunization or vaccination) are known in the art and/or described herein. In some embodiments, the immune response comprises a T cell-mediated response (e.g., peptide-specific response such as a proliferative response or a cytokine response). Antigenic compositions can be administered in a number of suitable ways, such as intramuscular injection, subcutaneous injection, intradermal administration and mucosal administration such as oral or intranasal. Additional modes of administration include but are not limited to intravenous, intraperitoneal, intranasal administration, intra-vaginal, intra-rectal, and oral administration. A combination of different routes of administration in the immunized subject, for example intramuscular and intranasal administration at the same time, is also contemplated by the disclosure.

Antigenic compositions may be used to treat both children and adults, including pregnant women. Thus a subject may be less than 1 year old, 1-5 years old, 5-15 years old, 15-55 years old, or at least 55 years old. Preferred subjects for receiving the vaccines are the elderly (e.g., >55 years old, >60 years old, preferably >65 years old), and the young (e.g., <6 years old, 1-5 years old, preferably less than 1 year old). Additional subjects for receiving the vaccines, SNAs, or compositions of the disclosure include immunocompromised subjects.

Administration can involve a single dose or a multiple dose schedule. In any of the aspects or embodiments, of the disclosure, administration comprises or consists of two doses. Multiple doses may be used in a primary immunization schedule and/or in a booster immunization schedule. In a multiple dose schedule the various doses may be given by the same or different routes, e.g., a parenteral prime and mucosal boost, a mucosal prime and parenteral boost, or a subcutaneous prime and a subcutaneous boost. Administration of more than one dose (typically two doses) is particularly useful in immunologically naive subjects or subjects of a hyporesponsive population (e.g., diabetics, or subjects with chronic kidney disease (e.g., dialysis patients)). In various embodiments, the second dose is administered about or at least about 2 weeks after the first dose. Multiple doses will typically be administered at least 1 week apart (e.g., about 2 weeks, about 3 weeks, about 4 weeks, about 6 weeks, about 8 weeks, about 10 weeks, about 12 weeks, or about 16 weeks). In some embodiments, multiple doses are administered from one, two, three, four or five months apart. Antigenic compositions of the present disclosure may be administered to patients at substantially the same time as (e.g., during the same medical consultation or visit to a healthcare professional) other vaccines.

In some embodiments, the hybrid peptide or SNA of the disclosure, or a combination thereof, is used to treat a disease. Thus, in some aspects, the disclosure provides methods of treating a disorder comprising administering an effective amount of hybrid peptide or SNA or combination thereof of the disclosure to a subject (e.g., a human subject) in need thereof, wherein the administering treats the disease. In various embodiments, the disease is a cancer. An “effective amount” of the hybrid peptide or SNA or a combination thereof, is an amount sufficient to, for example, treat, ameliorate, and/or prevent the disease. In other embodiments, an “effective” amount of the hybrid peptide or SNA, or a combination thereof, is an amount effective to induce an immune response in the subject, without causing significant, adverse side effects in the subject. In various embodiments, the cancer is breast cancer, peritoneum cancer, cervical cancer, colon cancer, rectal cancer, esophageal cancer, eye cancer, liver cancer, pancreatic cancer, larynx cancer, lung cancer, skin cancer, ovarian cancer, prostate cancer, stomach cancer, testicular cancer, thyroid cancer, brain cancer, or a combination thereof.

### EXAMPLES

Development of de novo hybrid linker-antigen peptides. To design a peptide sequence that would release an antigen peptide in its native state, data from the Proteomic Identification of Protease Cleavage Sites (PICS) database was used to computationally screen linker designs for their selectivity and specificity of Cathepsin S.⁷ In brief, this database was generated utilizing a library of peptide substrates derived from the proteome of fully sequenced species, which were generated from digestion of cell lysates with proteases which exclusively cleave at the C-terminus of particular amino acids, such as GluC, trypsin, or chymotrypsin. Following both sulfhydryl and amine protections, the library was digested by the desired protease and products extracted though biotin-streptavidin purification methods. Finally, the cleavage products were purified and sequenced via liquid chromatography tandem mass spectrometry (LC-MS/MS), and the adjoining side was derived bioinformatically from the proteome used. Importantly, the positional frequencies of these databases were normalized to the natural amino acid abundance of the initial library, providing the protease's selectivity and specificity. This database also provided profiling of amino acids along both sides of a cleavage site.⁸ Initial linker development was conducted with the ubiquitous ovalbumin system as a model, with the MHC class I restricted epitope SIINFEKL (OVA₂₅₇₋₂₆₄) serving as the antigen.

Given that the substate-binding pocket of Cathepsin S is indicated as S4-S1/S1′-S4′, and to generate the desired linker sequence the antigen would attribute to the S1′-S4′ positions, there are 160,000 distinct linker combinations using the 20 naturally occurring amino acids. Therefore, all 160,000 different combinations were screened computationally using the PICS database as a screening tool (FIG. 1B). A “cleavage probability” value was defined herein, which is the summation of the PICS normalized positional occurrence for each amino acid throughout the peptide sequence normalized to the difference between the maximum and minimum achievable values within the database. In addition, the selectivity of the peptide to Cathepsin S over other Cathepsins, specifically B and L in the PICS database, was calculated by comparing the ratio of the Cathepsin S “cleavage probability” to that of the other Cathepsins using their individual databases. Cutoff values used for screening the “cleavage probability” and selectivity of peptides for Cathepsin S over Cathepsin B and Cathepsin L were established using a literature-derived peptide (GRWPPMG-LPWEKRD) which has shown to have a single cleavage site between G-L, and is highly specific for Cathepsin S.⁶ Finally, the Gravy index of each individual peptide was calculated as a method to screen for peptide hydrophobicity, using a gravy score of 0.3 as a cutoff to avoid peptides which are highly hydrophobic.

After the extensive computational screening, a family of peptides sharing the sequence QXME, where X can be any one of the 20 naturally occurring amino acids, was selected as the most optimum sequence. Within this family of peptides, three were synthesized using standard solid-state peptide synthesis practices to test their ability to be cleaved by Cathepsin S at the desired cleavage site between E-S, where the peptides contained the sequence GQIMESIINFEKL, GQSMESIINFEKL, and GQPMESIINFEKL (FIG. 1C). Notably, G was added to the N-terminus of the peptides to inhibit the Q residues located at the N-termini from undergoing spontaneous intramolecular cyclization, resulting in the formation of pyroglutamic acid residues, as G in the P5 position had little effect on peptide screening. It was hypothesized that the difference in hydrophobicity of the amino acid in the P3 position, I>S>P, would affect the ability of the linkers to successfully bind to the pocket of Cathepsin S, therefore altering cleavage rate kinetics. This is supported though computationally modeling the ligand binding of the P4-P4′ positions of the hybrid linker-antigen peptides to the binding site of Cathepsin S (1 NPZ) utilizing the Attracting Cavities docking software (FIG. 1D). For clusters which allow the cleavage site between E-S to be within 5 Å distance from the catalytic cysteine for nucleophilic attack according to literature,¹⁰ QPME-SIIN peptide exhibited the lowest AC score (32.64) followed by QSME-SIIN (201.38) and QIME-SIIN (785.26). Thus, this suggested that the identity of the amino acid in the P3 position has a direct impact on substrate-ligand binding, with QPME-SIIN exhibiting the most favorable docking configuration to the active site followed by QSME-SIIN and QIME-SIIN.

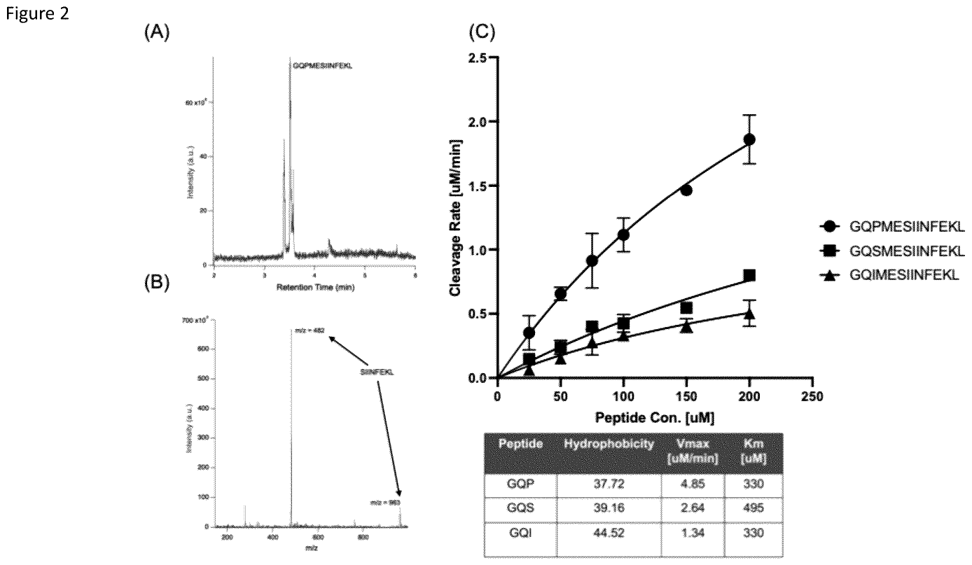

Characterization of Peptide Cleavage and Kinetics. After identifying peptide linker sequences though a high-throughput analysis, the ability of the hybrid linker-antigen peptides to release SIINFEKL after proteolysis by human Cathepsin S was investigated in a stopped-enzyme assay. Peptides [200 μM] were incubated with active Cathepsin S [10 nM] for 30 min, after which the Cathepsin S was inactivated through the addition of trifluoroacetic acid. The samples were then injected into a UPLC-MS where cleavage products were separated with a C18 column, and products were identified based on the resulting mass spectra. FIG. 2A displays a representative total ion chromatogram (TIC) of cleavage conducted with the GQPMESIINFEKL hybrid peptide, although GQSMESIINFEKL and GQIMESIINFEKL peptides exhibited similar TIC profiles. A peak corresponding to uncleaved peptide was observed along with a single peak which eluted at shorter retention times, which suggested a single product. Mass spectra traces (FIG. 2B) show m/z=482 and m/z=963 which corresponded to a doublet and singlet of SIINFEKL (MW=963.1 g/mol), which suggested that the peptide linkers are able to be cleaved at the desired site with high specificity. Importantly, the absence of additional peaks within the TIC and mass spectra plot suggested that no off-site cleavage occurs during proteolysis of the hybrid linker-antigen peptides, and that only a single cleavage site between E-S is achieved. It should be noted that a peak corresponding to GQPME was not observed in the TIC, which is likely due to the hydrophilicity of the peptide sequence and its inability to bind strongly to the C18 column.

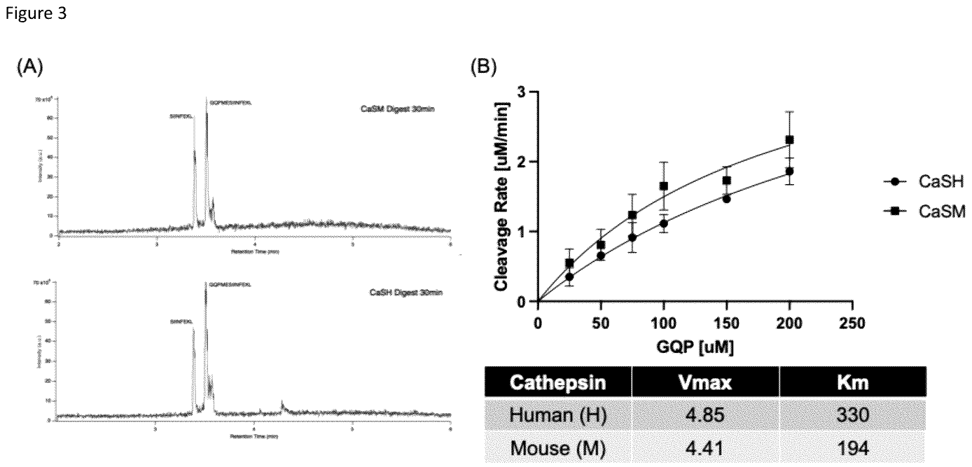

In addition to characterizing the resulting cleavage products, proteolysis kinetics of the hybrid linker-antigen peptides were fit using Michaelis-Menten kinetics (FIG. 2C). Resulting fits suggested that Cathepsin S most effectively cleaved the GQPME linker (Vₘₐₓ=4.85 μM/min), followed by the GQSME linker (Vₘₐₓ=2.64 μM/min), and is least effective at cleaving the GQIME linker (Vₘₐₓ=1.34 μM/min). Such results supported the hypothesis that tuning the hydrophobicity at the P3 position by altering the amino acid identity can provide temporal control over antigen release from the hybrid linker-antigen peptides in the presence of Cathepsin S, and the results fell in-line with the in silico docking predictions. Additionally, due the high homology (>75% according to Expasy SIM Alignment Tool for Protein Sequences) between human (1 NPZ) and murine (4BQV) Cathepsin S, the two proteases exhibited a singular cleavage site between E-S as demonstrated with digestion of GQPMESIINFEKL (FIG. 3A). Michaelis-Menten kinetics of GQPMESIINFEKL cleavage share similar fits, Vₘₐₓ=4.85 μM/min vs Vₘₐₓ=4.41 μM/min and Kₘ=300 μM vs. Kₘ=194 μM for human and murine Cathepsin S respectively (FIG. 3B). Overall, the similarities in degradation between human and mouse Cathepsin S suggested the peptides are applicable to both human and murine-based systems.

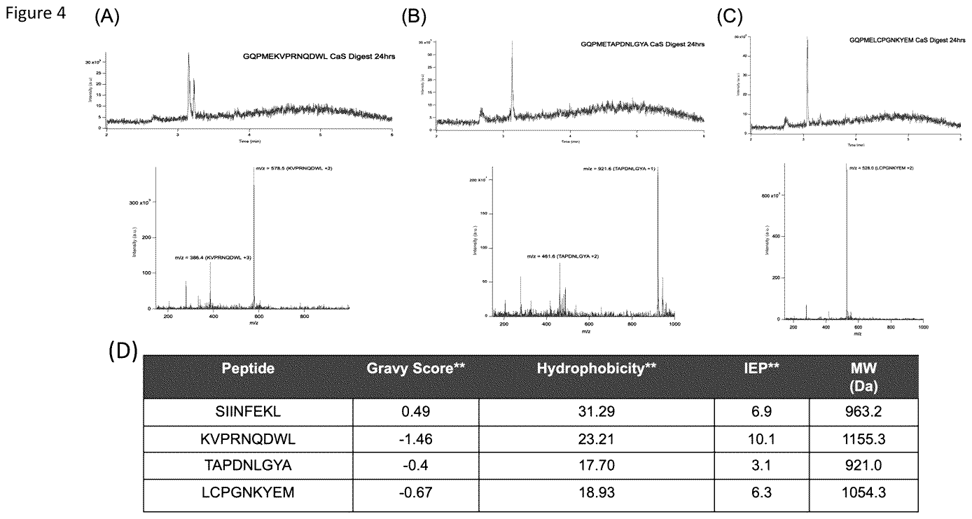

In addition to the investigation of the cleavage products of the hybrid linker-antigen peptides using the model SIINFEKL antigen, the applicability of the linker sequences to be applied to antigens with different sequences, charges, and hydrophobicity was explored. Here, literature established MCH-I epitopes KVPRNQDWL (gp100₂₅₋₃₃), TAPDNLGYA (TRP-1₄₅₅₋₄₆₃), and LCPGNKYEM (MART-1-M27) containing the GQPME linker were incubated [200 μM] with Cathepsin S [10 nM] for 24 hrs, and the resulting cleavage products were verified via UPLC-MS (FIG. 4A-4C). From the TIC and mass spectrum corresponding to the product peak, it can be suggested that the linkers are applicable to antigen sequences outside of the model SIINFEKL sequence, with different degrees of hydrophobicity, charge, and primary sequence. Much like what was observed with the SIINFEKL antigen, the KVPRNQDWL, TAPDNLGYA, and LCPGNKYEM antigens all exhibit a singular product peak within the TIC which corresponds to the antigen in its unmodified state. It should be noted that a small second peak can be observed at retention times below 3 min, which corresponds to the peptide linker GQPME which is likely observable due to the high degree of cleavage from the long incubation time. It should also be noted that there still remains some hybrid linker-antigen peptide even after 24 hr digest for KVPRNQDWL, suggesting that although the hybrid linker-antigen peptides are susceptible to cleavage, the antigen sequence likely has an effect on the rates of kinetics which is expected. However, overall this data suggested that the de novo linkers are applicable to different antigen sequences, while allowing the antigens to be released after proteolysis by Cathepsin S.

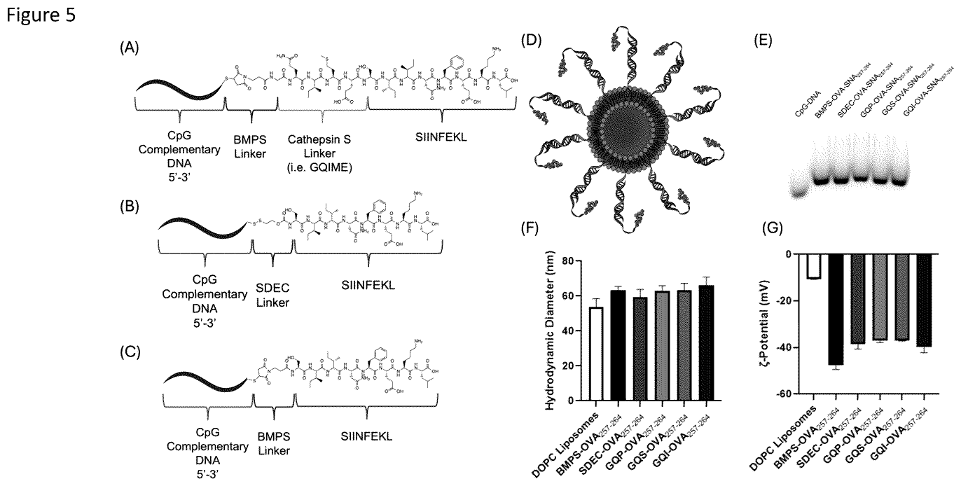

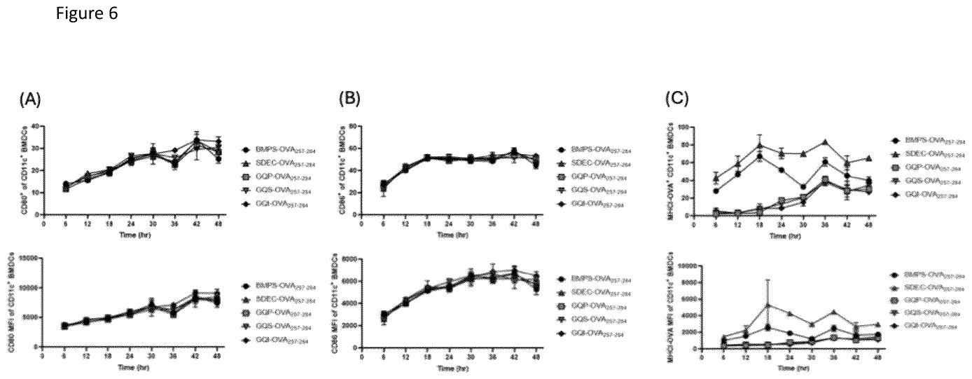

Hybrid Linker-Antigen Peptide SNA Synthesis and Characterization. To investigate the ability of the hybrid linker-antigen peptides to illicit an immune response, they were formulated into liposomal spherical nucleic acid (SNA) vaccines synthesized using the hybridized SNA model as previously established.² Hybrid linker-antigen peptides were chemically conjugated to the 3′ end of the complementary class B CpG murine TLR9 ligand (comp CpG 1826). The synthesis was accomplished by modifying the 3′ end of the DNA with a thiol, and attaching the hybrid peptide to the DNA via a non-cleavable N-β-maleimidopropyl-oxysuccinimide ester (BMPS) small molecule linker attached to the N-terminus of the hybrid peptide (FIG. 5A). In addition, a positive control containing OVA₂₅₇₋₂₆₄ conjugated to the complementary strand via a redox-sensitive “traceless” (SDEC) linker (FIG. 5B), and a non-cleavable BMPS linker (FIG. 5C), as a negative control were also synthesized. The peptide-DNA conjugates were purified via preparative polyacrylamide gel electrophoresis and products verified by MALDI-ToF, and further hybridized to class B CpG murine TLR9 ligand (CpG 1826) modified with a cholesterol moiety on the 3′ end by slow cooling. SNAs were synthesized by incubating 1,2-dioleoyl-snglycero-3-phosphocholine (DOPC) liposomes (~50 nm diameter) with the duplexed SNA strands to allow for 3′ cholesterol to intercalate into the bilayer of the liposome to form the oligonucleotide shell, (FIG. 5D).

SNA formation was characterized by gel electrophoresis, FIG. 5E, DLS, FIG. 5F, and zeta (ζ)-potential measurements, FIG. 5G. The slower electrophoretic mobility observed by gel electrophoresis, indicating the formation of a larger structure due to intercalation of DNA into the liposomal bilayer, is indicative of SNA formation. Importantly, at a loading density of 75 oligonucleotides per liposome there is no observable free DNA in the SNA sample bands suggesting the hybrid linker-antigen peptides do not inhibit the ability of the DNA to intercalate into the liposomal bilayer. The size increase observed via DLS from 53.8±4.7 nm (bare liposomes) to 63.0±2.4 nm (averaged across all SNA types) in 1×PBS is representative of SNA formation, and importantly, the similarities in hydrodynamic diameter of SNAs containing hybrid linker-OVA₂₅₇₋₂₆₄ peptides to that of SNAs with the OVA₂₅₇₋₂₆₄ antigen suggested the addition of amino acids in the peptide linker does not alter particle stability or size. Finally, SNA samples exhibited a significant decrease in ζ-potential compared to bare liposomes, suggestive of localization of negatively charged oligonucleotides onto the liposomal surface. It should be noted that the ζ-potential measurements for SNAs formulated with the GQPME, GQSME, and GQIME hybrid linker-OVA₂₅₇₋₂₆₄ peptides did exhibit similar ζ-potential measurements to that of the BMPS-OVA₂₅₇₋₂₆₄ and SDEC-OVA₂₅₇₋₂₆₄ SNAs even though the peptides exhibited slightly different isoelectric points (SIINFEKL IEP=6.9; GQPMESIINFEKL, GQSMESIINFEKL, and GQIMESIINFEKL IEP=4.3). However, it is likely that the highly negative charge of the oligonucleotide shell in the SNA architecture conceals any slight changes in the overall charge of the conjugated peptide. Overall, the incorporation of hybrid linker-antigen peptides into the formulation of SNA nanoconstructs do not cause significant changes in the ability to formulate SNAs.

In vitro DC and T Cell Activation by SNAs. The ability to invoke a robust immune response is highly dependent on interactions between activated dendritic cells (DCs) and T cells, where DCs present antigens onto MHC class I complexes along with the expression of costimulatory markers.¹¹ Therefore, the ability of the SNA constructs to activate DCs through the surface expression of costimulatory markers CD80 and CD86 was investigated, along with the presentation of SIINFEKL on MHC class I complexes. Bone-marrow derived dendritic cells (BMDCs) were pulsed with SNAs for 1 hr (5 μM by CpG and OVA₂₅₇₋₂₆₄) and subsequently washed to remove SNAs from the cell medium. DCs were then incubated in fresh medium for up to 48 hrs, with timepoints taken every 6 hrs. Following incubation, cells were stained and analyzed by flow cytometry. Overall, CD80 and CD86 expression profiles across the 48 hr time course were similar for each SNA construct, independent on linker-identity, with the maximum expression of both CD80 and CD86 taking place ~24-30 hrs after treatment (FIG. 6A-6B). The similar levels of DC activation across SNAs suggests that linker identity, i.e disulfide, non-cleavable, or peptide-based, does not affect the ability of the SNAs to stimulate TLR9. The most significant difference between the different SNAs was observed with the presentation of OVA₂₅₇₋₂₆₄ on MHC class I over the 48 hr time course (FIG. 6C). Similar to previous observations using the SDEC reduction labile linker¹, presentation begins in as little as ~6 hrs after treatment and reaches a maximum within ~18 hrs with ~80% of measured cells expressing OVA₂₅₇₋₂₆₄ on MHC class I complexes. Furthermore, DCs continue to present OVA₂₅₇₋₂₆₄ over the 48 hr time course with >60% of cells expressing OVA₂₅₇₋₂₆₄ at 48 hrs. SNAs conjugated with BMPS-OVA do result in OVA₂₅₇₋₂₆₄ presentation following similar kinetics to that of the reducible SDEC linker, but with a lower overall expression profile at each timepoint. While the BMPS linker is not readily reducible, it is not expected to be an indefinitely stable conjugate as similar linkers are known to degrade over time but at much slower kinetic rates compared to redox-responsive chemistries.¹²

Surprisingly, the presentation kinetics of OVA₂₅₇₋₂₆₄ on MHC class I complexes differ drastically between the Cathepsin S hybrid linker-OVA₂₅₇₋₂₆₄ SNAs and both the SDEC-OVA₂₅₇₋₂₆₄ and BMPS− OVA₂₅₇₋₂₆₄ SNA. While SDEC-OVA₂₅₇₋₂₆₄ SNA results in OVA₂₅₇₋₂₆₄ expression in as little as 6 hrs, the OVA₂₅₇₋₂₆₄ expression with hybrid linker-peptide SNAs is near negligible until around ~24 hrs. Even after 24 hrs, the maximum % cells which contain OVA₂₅₇₋₂₆₄ on MHC class I reaches a maximum of ~40%, which is only approximately half the maximum % cells which express OVA₂₅₇₋₂₆₄ on MHC class I with the SDEC linker. The increase in OVA₂₅₇₋₂₆₄ presentation of the hybrid linker-OVA₂₅₇₋₂₆₄ SNAs at later timepoints could be caused by an upregulation of Cathepsin S expression, as previous work has demonstrated that Cathepsin S mRNA expression is significantly enhanced within DC subsets upon activation of toll-like receptors, including TLR9.¹³ The significant differences between the OVA₂₅₇₋₂₆₄ presentation kinetics suggests that SNAs containing the hybrid linker-OVA₂₅₇₋₂₆₄ conjugates undergo different routes of intracellular processing, potentially suggesting different intracellular antigen processing pathways.

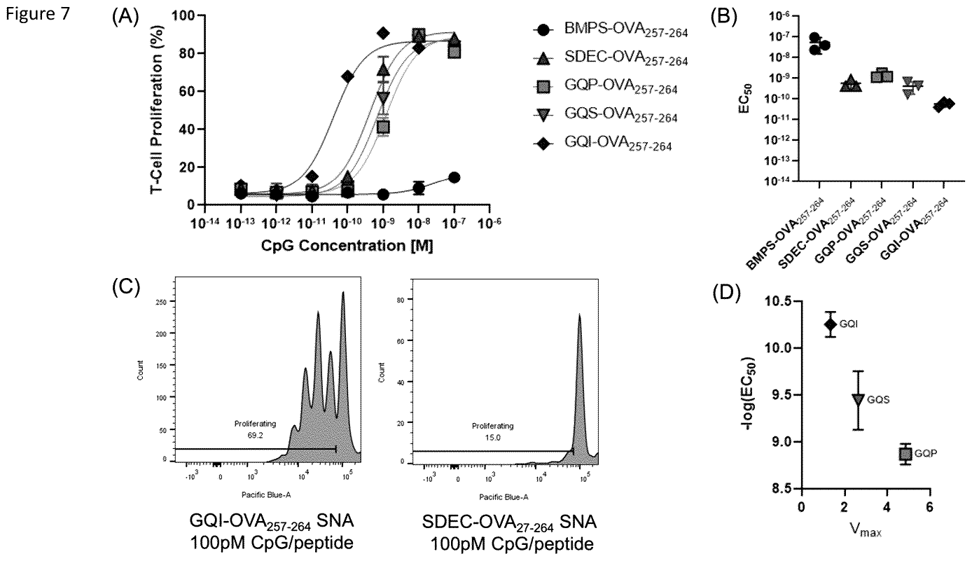

The efficacy of the SNA compositions to elicit an immune response was investigated by measuring the proliferation of CD8⁺ T cells after SNA treatment in vitro, as T cell proliferation resulting from stimulation of antigen presenting cells is indicative of a robust immune response.¹⁴ Splenocytes containing T cells which specifically recognize the SIINFEKL epitope were derived from OT-1 mice, and treated with all 5 SNA treatments. Here, the BMPS-OVA₂₅₇₋₂₆₄ and SDEC-OVA₂₅₇₋₂₆₄ SNA served as control groups which have been shown to previously result in low and robust T cell activation respectively.¹ The SNA treatment groups were incubated with surface-stained (amine-reactive eFluor 450) whole splenocytes from OT-1 mice for 72 hrs, and following stimulation the percent of proliferating CD8⁺ T cells was quantified by flow cytometry. Using a range of treatment concentrations, each performed in duplicate, dose-responsive curves were fit to T-cell proliferation for each SNA group (FIG. 7A). Interestingly, the SNA containing the GQI-OVA₂₅₇₋₂₆₄ hybrid linker-antigen peptide exhibits the highest potency (EC₅₀=57.6 μM) of the compositionally identical but chemically unique SNA designs (FIG. 7B). This potency is ~10× higher than the potency of the SDEC-OVA₂₅₇₋₂₆₄ SNA (EC₅₀=577 μM), which is evident when comparing the proliferation profiles between the GQI-OVA₂₅₇₋₂₆₄ and SDEC-OVA₂₅₇₋₂₆₄ treatments at 100 μM by CpG DNA/peptide (FIG. 7C). Importantly, the hybrid linker-OVA₂₅₇₋₂₆₄ SNAs exhibit significantly lower EC₅₀ than the BMPS-OVA₂₅₇₋₂₆₄ constructs, demonstrating that the enhanced potency stems from the Cathepsin S cleavable linker sequence (GQI-OVA₂₅₇₋₂₆₄ SNA ~1000× higher potency than the BMPS-OVA₂₅₇₋₂₆₄SNA (EC₅₀=53.7 nM)). The differences between the EC₅₀ of the hybrid linker-antigen peptide SNAs, GQI-OVA₂₅₇₋₂₆₄ (EC₅₀=57.6 μM) GQS-OVA₂₅₇₋₂₆₄ (EC₅₀=422 μM) and GQP-OVA₂₅₇₋₂₆₄ (EC₅₀=1.39 nM) respectively, suggest that kinetics of peptide cleavage has a direct impact on the resulting immune activation. This is surprising considering the similarities between the kinetic profiles of OVA₂₅₇₋₂₆₄ on MHC class I between the constructs, which does not show significant differences. The correlation between cleavage kinetics and immune activation (FIG. 7D), suggest that potency is negatively correlated with maximum rate of proteolysis, suggesting that timing of antigen release is more important than the amount of antigen that is released with the peptide-based linkers. This is opposite of the findings previously established with redox-responsive chemical linkers utilizing the SNA format.¹ The overall potency of the hybrid linker-OVA₂₅₇₋₂₆₄ SNAs is surprising considering the significant delay in OVA₂₅₇₋₂₆₄ presentation of the hybrid linker-peptide antigens in FIG. 6C compared to the SDEC-OVA₂₅₇₋₂₆₃ construct. As it currently stands, it is believed that this higher potency could stem from a more synergistic delivery of both antigen and adjuvant in a manner which results in robust immune activation, but this work is currently ongoing.

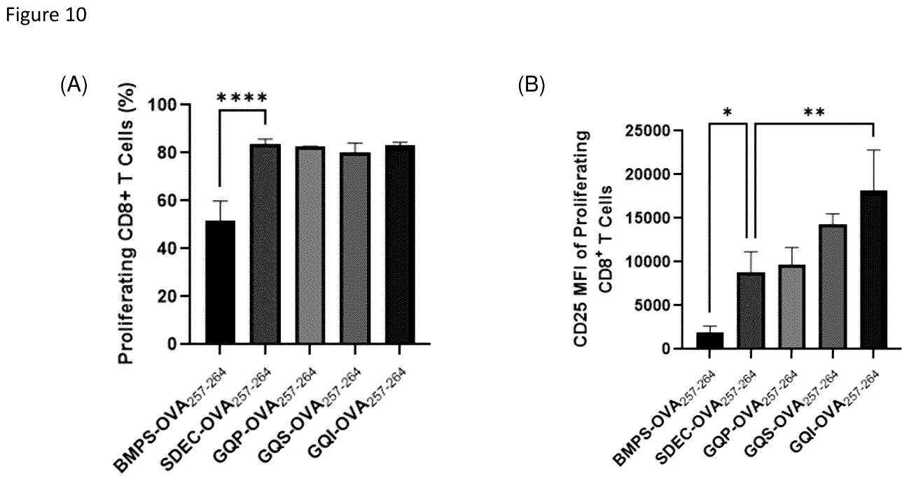

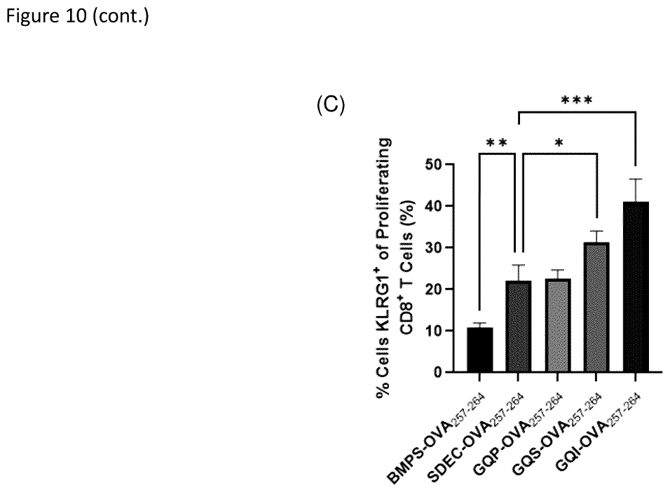

To further characterize functional T cell responses, bone marrow-derived dendritic cells were pulsed with SNA formulations (1 hour, 1 μM CpG/peptide) and co-cultured with OT-I splenocytes. SNAs comprising cleavable linkers (SDEC-OVA₂₅₇₋₂₆₄, GQP-OVA₂₅₇₋₂₆₄, GQS-OVA₂₅₇₋₂₆₄ and GQI-OVA₂₅₇₋₂₆₄) induced proliferation in approximately 80% of CD8⁺ T cells, (FIG. 10A), with cells undergoing multiple rounds of division. In contrast, the non-cleavable BMPS-OVA₂₅₇₋₂₆₄ SNA induced proliferation in approximately 50% of CD8⁺ T cells with reduced division depth. Given the comparable extent of proliferation among SNAs comprising cleavable linkers, the activation state of the responding CD8⁺ T cells was further evaluated by assessing surface expression of the IL-2 receptor a chain (CD25) (FIG. 10B). Hybrid linker-antigen SNAs induced elevated CD25 expression relative to the redox-responsive SDEC-OVA₂₅₇₋₂₆₄ comparator, with statistically significant enhancement observed for GQI− OVA₂₅₇₋₂₆₄, indicating a heightened activation state associated with linker-dependent antigen presentation.

Differentiation toward a cytotoxic effector phenotype was assessed by quantifying expression of KLRG1 (FIG. 10C). SDEC-OVA₂₅₇₋₂₆₄ treatment resulted in approximately 20% KLRG1⁺ CD8⁺ T cells, representing a two-fold increase relative to the non-cleavable BMPS-OVA₂₅₇₋₂₆₄ construct. The GQI-OVA₂₅₇₋₂₆₄ SNA induced the higher proportion of KLRG1⁺ CD8⁺ T cells, approximately two-fold greater than SDEC-OVA₂₅₇₋₂₆₄ followed by GQS-OVA₂₅₇₋₂₆₄ and GQP-OVA₂₅₇₋₂₆₄. These data demonstrated that hybrid linker-antigen peptide SNAs produced antigen-specific CD8⁺ T cells exhibiting enhanced proliferation and effector differentiation relative to redox-responsive and non-cleavable comparator constructs.

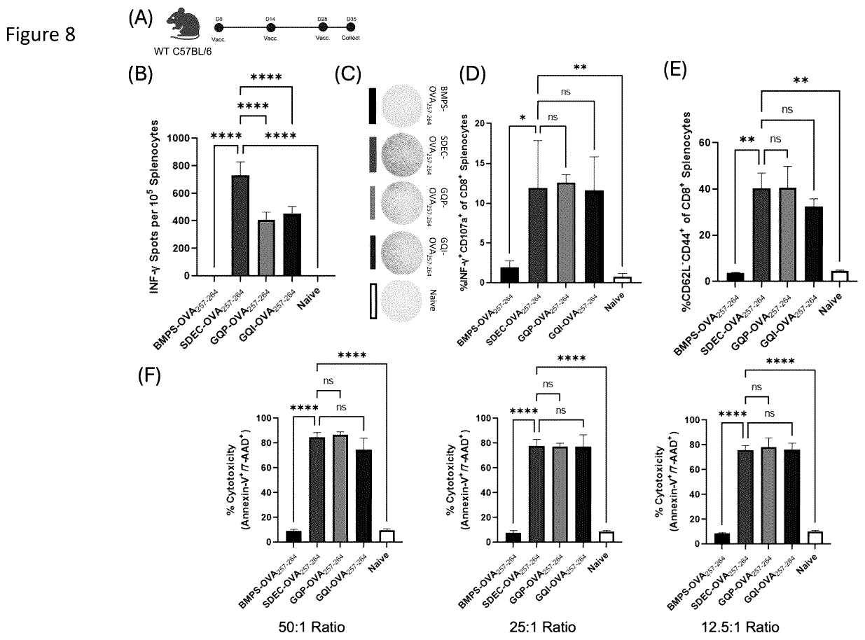

In vivo Immune Activation and Tumor Studies. Given the stark differences in antigen presentation kinetics and immune activation in vitro, the ability of the hybrid linker-antigen peptides to result in a more immunogenic construct in vivo was investigated with a focus on the ability of the SNAs to prime antigen specific T-cells over time. For initial in vivo studies the best and worst performing hybrid linker-antigens determined from in vitro assays, GQP-OVA₂₅₇₋₂₆₄ and GQI OVA₂₅₇₋₂₆₄ respectively, were compared to the reducible SDEC-OVA₂₅₇₋₂₆₄ conjugate and non-cleavable BMPS-OVA₂₅₇₋₂₆₄ control. C57BL/6 mice (n=3 per group), were immunized with a total of 3 injections biweekly (6 nmol of OVA₂₅₇₋₂₆₄ and 6 nmol of CpG DNA) (FIG. 8A) though subcutaneous injections in the abdomen to promote drainage to the inguinal lymph nodes.^{2, 15} A week following the third injection splenocytes were collected to assess induction of specific immune responses towards the OVA₂₅₇₋₂₆₄ antigen. Antigen specific secretion of the proinflammatory cytokine interferon gamma (INF-γ) though an enzyme-linked immunosorbent spot (ELISpot) assay (FIG. 8B-8C) showed that animals immunized with the SNAs containing the reducible SDEC linker resulted in the highest count of spot forming cells (SFCs). SDEC-OVA₂₅₇₋₂₆₄ SNA treatment resulted in ~244-fold enhancement in SFCs compared to the non-cleavable BMPS-OVA₂₅₇₋₂₆₄ SNA control, which did not show significant improvement of T cell priming compared to naïve mice. The SDEC-OVA₂₅₇₋₂₆₄ SNA also resulted in ~1.76-fold enhancement in formation of SFCs compared to the hybrid linker-antigen peptide containing SNAs, which both resulted in similar levels of SFC development. This suggests that the differences observed in the potency of the hybrid linker-antigen constructs in their ability to activate cytotoxic CD8+T in vitro are lost over the five-week immune buildup. However, both the GQP-OVA₂₅₇₋₂₆₄ and GQI OVA₂₅₇₋₂₆₄ SNA constructs led to significantly higher formation of SFCs compared to the non-cleavable BMPS control showing the importance in the cleavability of the peptide linker sequence. Although results from ELISpot suggest heightened immune activation from immunization with SDEC-OVA₂₅₇₋₂₆₄ SNAs, this trend did not materialize for splenocyte single cell analysis of CD8⁺ T cells expressing intracellular INF-γ and the degranulation marker CD107a (FIG. 8D). Although SDEC-OVA₂₅₇₋₂₆₄ SNA led to significantly higher intracellular INF-γ compared to the naïve and non-cleavable BMPS-OVA₂₅₇₋₂₆₄ controls, there was no statistically significant difference compared to either GQP-OVA₂₅₇₋₂₆₄ or GQI-OVA₂₅₇₋₂₆₄ SNAs. SDEC-OVA₂₅₇₋₂₆₄, GQP-OVA₂₅₇₋₂₆₄, and GQI-OVA₂₅₇₋₂₆₄ SNAs all resulted in similar elevated levels of polyfunctional T cells in the splenocytes, which are considered potent against chronic infections and tumors.¹⁵ Additionally, these treatments resulted in similar levels of CD8⁺ T cells with effector memory phenotype (CD44⁺/CD62L⁻) and significantly higher than both naïve mice and mice immunized with BMPS− OVA₂₅₇₋₂₆₄ SNAs (FIG. 8E). Finally, the ability of activated and isolated CD8⁺ T cells to kill target cancer cells which express ovalbumin (E.G7-OVA) was evaluated (FIG. 8F) by measuring E.G7-OVA cells which were double-positive for the apoptotic and necrotic markers Annexin V and 7-AAD respectively. Across all T-cell to E.G7-OVA cell ratios tested, T-cells from mice immunized with the SDEC-OVA₂₅₇₋₂₆₄, GQP-OVA₂₅₇₋₂₆₄, and GQI-OVA₂₅₇₋₂₆₄ SNAs exhibited high levels of target cancer killing efficacy ex vivo, with no statistically significant differences between the three groups. This killing efficacy was significantly higher than the BMPS-OVA₂₅₇₋₂₆₄ and naïve control, which both exhibited ineffective cancer cell killing. Overall, this in vivo assessment suggests that SNAs containing hybrid linker-antigen peptides result in robust immune activation of antigen specific T-cells, on par to that of previously established SNA designs containing redox-responsive linkers over long-term immune stimulation.

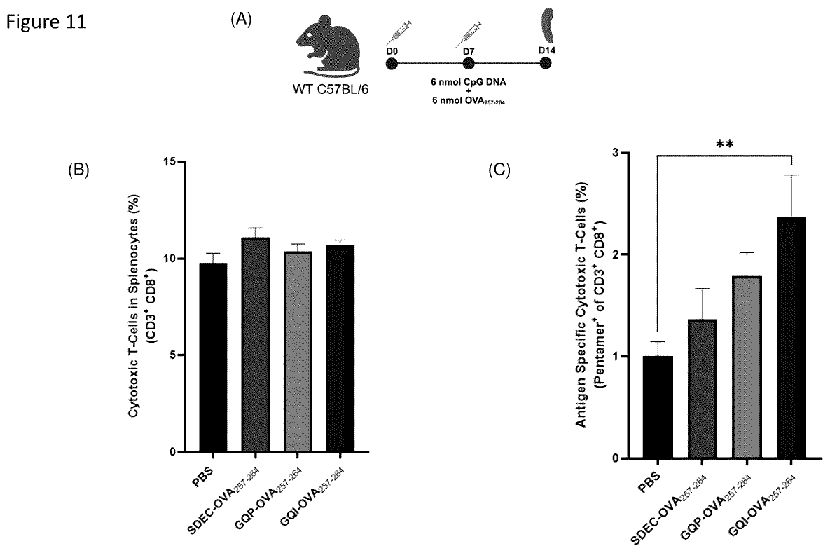

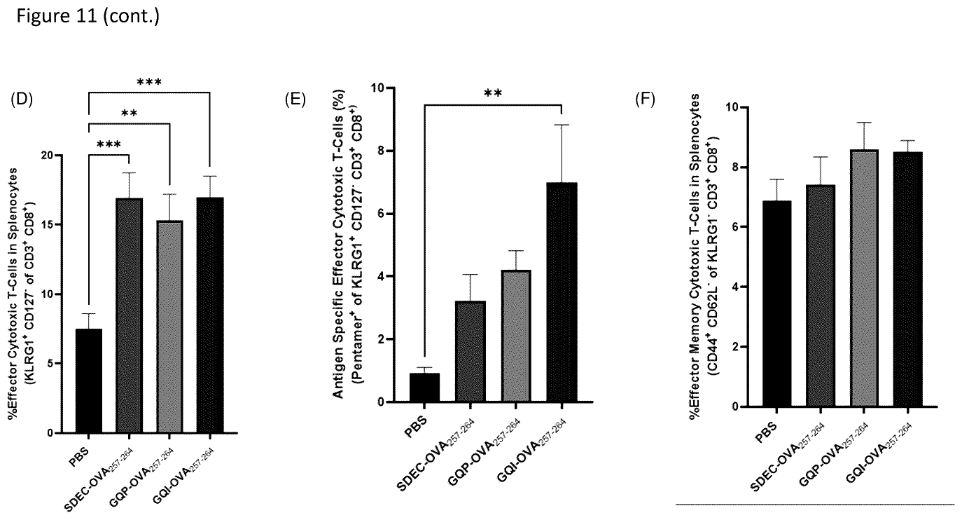

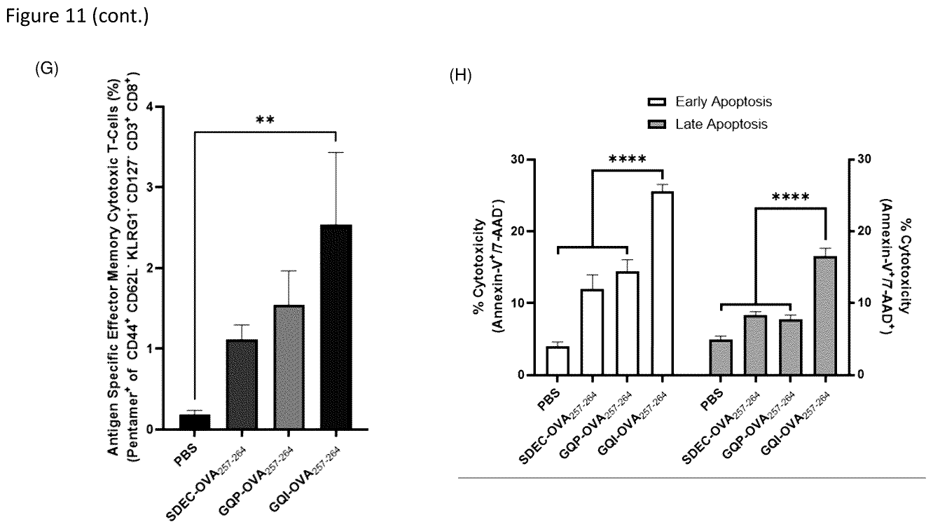

The foregoing experiments were performed again using a therapeutic schedule that is more representative of treatment schedules employed in murine antitumor models. The results showed that SNAs containing hybrid linker-antigen peptides showed an even more robust immune response and earlier antigen specific response relative to the experiments described above and depicted in FIG. 8. As above, based on the observed differences in antigen presentation kinetics and immune activation in vitro, the ability of hybrid linker-antigen peptide SNAs to induce antigen-specific immune responses in vivo was evaluated. The most potent and least potent hybrid linker-antigen SNA constructs were compared to a redox-responsive SNA comparator. C57BL/6 mice were immunized subcutaneously in the abdominal region with two weekly doses of SNAs comprising 6 nmol CpG DNA and 6 nmol OVA₂₅₇₋₂₆₄ peptide per injection (FIG. 11A). One week following the second immunization, splenocytes were harvested to assess antigen-specific T cell responses. Total splenic CD8+ T cell frequencies were comparable across all treatment groups (FIG. 11B). However, mice treated with the GQI-OVA₂₅₇₋₂₆₄ SNA exhibited a statistically significant increase in the frequency of OVA₂₅₇₋₂₆₄-specific CD8⁺ T cells relative to control groups (FIG. 11C). Treatment with GQP-OVA₂₅₇₋₂₆₄ and SDEC-OVA₂₅₇₋₂₆₄ SNAs resulted in moderate, non-significant increases in antigen-specific CD8⁺ T cell frequency.

Phenotypic analysis of CD8⁺ T cell subsets demonstrated increased frequencies of short-lived effector T cells (KLRG1⁺/CD127⁻) within the total CD8⁺ splenocyte population for all SNA-treated groups (FIG. 11D). Notably, only the GQI-OVA₂₅₇₋₂₆₄ SNA induced a statistically significant expansion of OVA₂₅₇₋₂₆₄-specific short-lived effector CD8⁺ T cells (FIG. 11E). SNA vaccination also increased the frequency of effector memory CD8⁺ T cells (CD44⁺/CD62L⁻/KLRG1⁻) within splenocyte populations (FIG. 11F). While increases in total effector memory cells were not statistically significant, GQI-OVA₂₅₇₋₂₆₄ treatment resulted in elevated frequencies of OVA₂₅₇₋₂₆₄-specific effector memory CD8⁺ T cells (FIG. 11G).

The cytotoxic function of vaccine-induced CD8⁺ T cells was assessed ex vivo using E.G7-OVA target cells. Target cell death was quantified by Annexin V and 7-AAD staining to verify early apoptotic (Annexin V⁺/7-AAD⁻) and late apoptotic or necrotic (Annexin V⁺/7-AAD⁺) populations (FIG. 11H). Splenocytes from mice vaccinated with GQI-OVA₂₅₇₋₂₆₄ SNAs mediated the highest level of antigen-specific target cell killing, with significantly increased proportions of both early and apoptotic E.G7-OVA cells relative to all comparator groups.

Collectively, these results demonstrated that SNAs comprising hybrid linker-antigen peptides, particularly Cathepsin S-cleavable GQI linkers, induced elevated frequencies of antigen-specific CD8+ T cells in vivo and promoted their differentiation into cytotoxic short-lived effector and effector memory subsets, resulting in enhanced antigen-specific tumor cell killing.

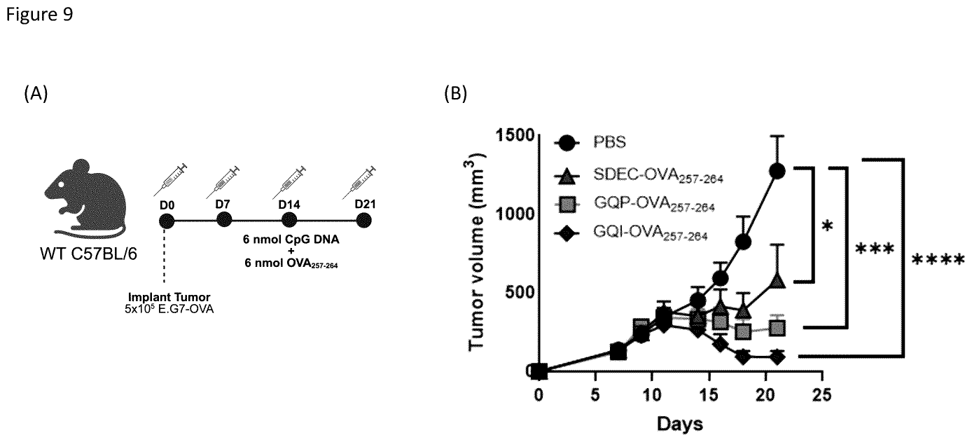

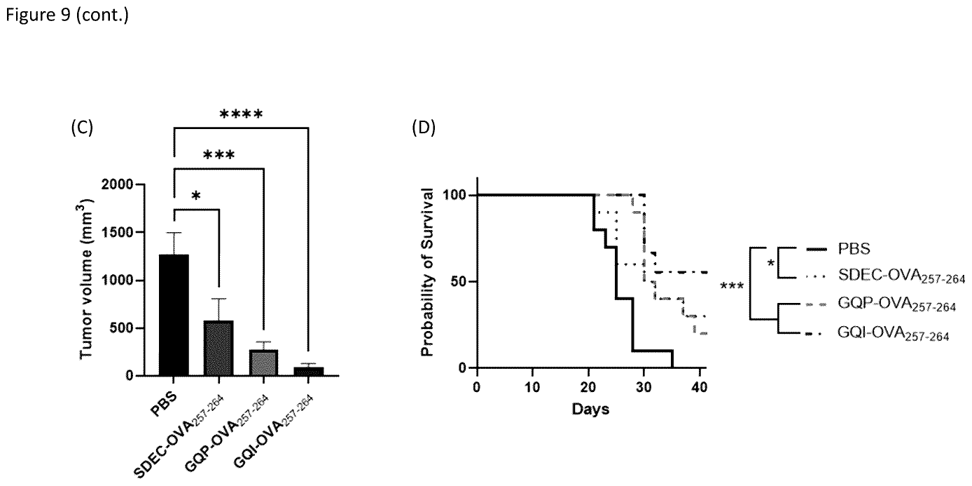

Motivated by the observation that SNAs containing the hybrid linker-antigen peptides result in a robust immune activation on par to that of the traditional reducible SNA formulation, the potency of the SNA constructs was evaluated against E.G7-OVA lymphoma tumors, which express the entire ovalbumin protein.¹⁶ C57BL/6 mice (n=9-10 mice per group) were inoculated subcutaneously with 5×10⁵ E.G7-OVA lymphoma cells in the right hind flank and, once tumors reached an average volume of approximately 130 mm³, treated with either SDEC-OVA₂₅₇₋₂₆₄, GQP-OVA₂₅₇₋₂₆₄, or GQI− OVA₂₅₇₋₂₆₄ SNA (6 nmol CpG DNA and 6 nmol OVA₂₅₇ peptide per dose) via weekly subcutaneous injection beginning seven days post-inoculation (FIG. 9A). Due to the previously established inability of BMPS-OVA₂₅₇₋₂₆₄ SNA to invoke a robust immune response in vivo and the conclusion that the potency of GQP-OVA₂₅₇₋₂₆₄ and GQI-OVA₂₅₇₋₂₆₄ SNAs stems from the peptide linker sequence, the construct was excluded from tumor studies. Additionally, due to similarities in immune activation observed in FIG. 8 between the SDEC-OVA₂₅₇₋₂₆₄, GQP-OVA₂₅₇₋₂₆₄, and GQI-OVA₂₅₇₋₂₆₄ SNAs, treatment regiments were not started until 7 days post tumor inoculation, when tumors were both palpable and could be visually observed under the skin, to amplify any differences in antitumor activity. All SNA treatments reduced tumor burden compared to saline-treated mice by day 21 (FIG. 9B). Surprisingly, the effectiveness in generating an antitumor response was significantly different for the compositionally identical but chemically distinct SNA constructs, resulting in 3.4-fold, 6.5-fold, and 15.3-fold reductions in tumor volume for SDEC-OVA₂₅₇₋₂₆₄, GQP-OVA₂₅₇₋₂₆₄, and GQI-OVA₂₅₇₋₂₆₄ SNAs respectively (FIG. 9C). The enhancement in antitumor response for the GQP-OVA₂₅₇₋₂₆₄ and GQI-OVA₂₅₇₋₂₆₄ constructs also led to a more significant enhancement in animal survival over the SDEC-OVA₂₅₇₋₂₆₄ SNAs, (FIG. 9D). Treatment with GQI-OVA₂₅₇₋₂₆₄ led to a 68% increase in median survival (42 days) over the PBS control group (25 days), whereas treatment with both GQP-OVA₂₅₇₋₂₆₄ and SDEC-OVA₂₅₇₋₂₆₄ only led to a 24% increase (31 days). Together, this suggests that SNA formulations which contain the hybrid linker-antigen peptides result in a more robust and higher antitumor activity compared to the redox-responsive SDEC-based SNA nanoconstructs and highlights the need to precisely control antigen processing in generating a beneficial therapeutic effect. Additionally, these studies suggest that the vacuolar cross-presentation pathway can play just as important of a role in regulating anticancer immunity in vivo, which to date has not been well characterized and understood.¹⁷

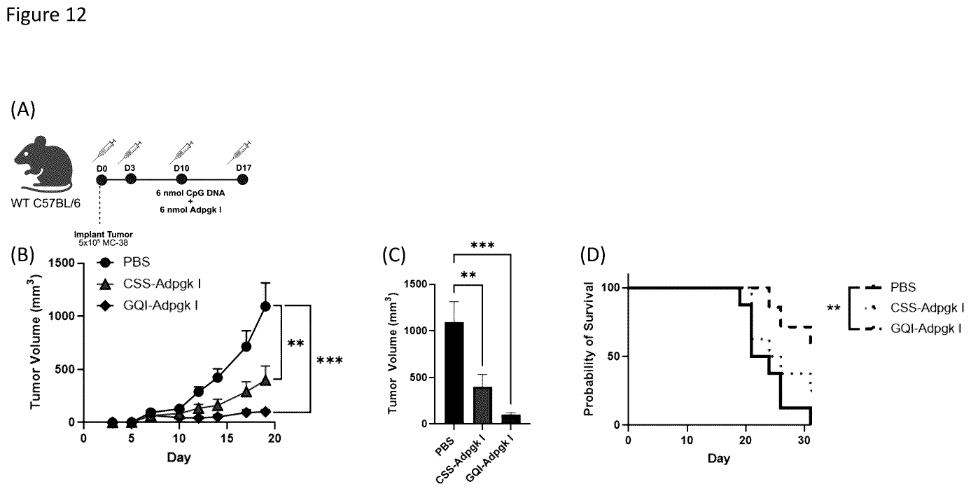

To assess applicability across distinct tumor types and antigenic contexts, hybrid linker-antigen SNAs were evaluated in the mutationally diverse MC-38 colon carcinoma model using the neoantigen Adpgk I (ASMTNMELM; SEQ ID NO: 15). C57BL/6 mice (n=7-8 mice per group) were inoculated subcutaneously with 5×10⁵ MC-38 cells and treated weekly with SNAs comprising either a redox-sensitive linker (CSS-Adpgk I) or a Cathepsin S-cleavable linker (GQI-Adpgk I) identified as highly potent in the E.G7-OVA model (6 nmol CpG DNA and peptide per dose) (FIG. 12A). Both SNA formulations significantly suppressed tumor growth relative to PBS controls (FIG. 12B). At day 17, CSS-Adpgk I treatment resulted in an approximate 2.8-fold reduction in tumor volume (FIG. 12C), but did not confer a statistically significant survival benefit, with only approximately 25% of treated animals reaching the 33-day study endpoint (FIG. 12D). In contrast, treatment with GQI-Adpgk I resulted in an approximate 10.7-fold reduction in tumor volume relative to PBS controls at day 17 and produced a marked survival benefit, with greater than 50% of animals reaching the study endpoint (FIG. 12D).

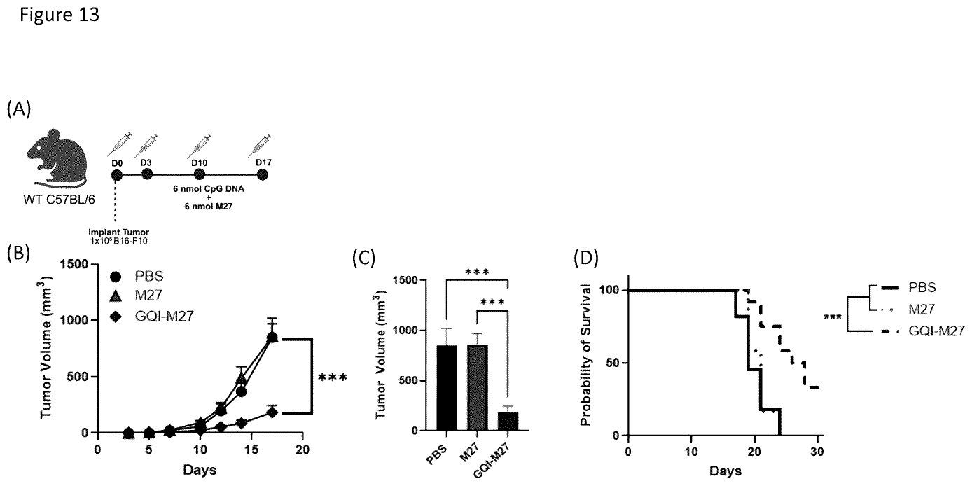

The hybrid linker-antigen SNA design was further evaluated in the aggressive and poorly immunogenic B16-F10 melanoma model using the reported M27 neoantigen (LCPGNKYEM; SEQ ID NO: 16). C57BL/6 mice (n=11-12 mice per group) were inoculated subcutaneously with 1×10⁵ B16-F10 cells and treated weekly with SNAs comprising either a redox-sensitive linker (M27-SNA) or a Cathepsin S-cleavable hybrid linker (GQI-M27 SNA), administered at 6 nmol CpG DNA and 6 nmol peptide per dose, without co-administration of immune checkpoint inhibitors (FIG. 13A). By day 17 post-inoculation, mice treated with GQI-M27 SNA exhibited an approximate 4.6-fold reduction in tumor volume relative to PBS-treated controls and redox-responsive M27 SNA-treated mice, the latter of which showed no significant therapeutic effect (FIGS. 13B-13C). This enhanced antitumor activity translated into improved survival outcomes (FIG. 13D), with the GQI-M27 SNA treatment increasing median survival by approximately 42% (27 days) relative to PBS controls (19 days). In contrast, M27 SNA treatment resulted in a non-significant survival increase of approximately 10% (21 days).

### Materials and Methods

All reagents were purchased commercially and used as received unless otherwise noted. Oligonucleotides were synthesized as described below with phosphoramidites and synthesis reagents purchased from Glen Research. Peptides were purchased from Northwestern's Peptide synthesis core, either purified (>95%) or on resin, and synthesized using standard peptide coupling chemistry. Chemicals were purchased from suppliers listed in parenthesis. Deionized water (diH₂O) was used from a Milli-Q system equipped with a 0.22 μm filter. C57BL/6 mice and C57BL/6-Tg(TcraTcrb)1100Mjb/J (OT1) female mice aged 6-8 weeks old were purchased from Jackson Laboratories. Mice were used in accordance with all national and local guidelines and regulations, and protocols were approved by the Institutional Animal Care and Use Committee at Northwestern University. E.G7-OVA and B16-F10 cells were purchased from ATCC and MC-38 cells were purchased from MilliporeSigma.

Docking Simulations. Docking simulations of the P4-P4′ segments of hybrid linker-OVA₂₅₇₋₂₆₄ peptides were conducted using the docking software Attracting Cavities. Cathepsin S cartesian coordinates from the 3D structure of human Cathepsin S (1NPZ) were used in docking simulations with a search space of 20.0 Å×20.0 Å×20.0 Å, centered around the C1P inhibitor present in the x-ray structure. Prior to docking simulations, the C1P inhibitor and all water molecules were removed. Models of docking clusters with Cathepsin S were generated and analyzed with UCSF Chimera Extensible Molecular Modeling System.

Peptide Cleavage Characterization. Recombinant human Cathepsin S (R&D Systems) or recombinant mouse Cathepsin S (Sino Biological) was incubated at 10 μg/ml in 20 mM Sodium Acetate, 100 mM NaCl, 1 mM EDTA, 5 mM DTT, pH 5.5 buffer at 37° C. with constant shaking (300 RPM) for 90 min to activate. 28 ng of active Cathepsin S was diluted to 10 nM with assay buffer (50 mM Citrate Phosphate, 100 mM NaCl, 1 mM EDTA, 5 mM DTT, pH 6.0, final volume 100 μl) containing purified peptide at concentrations of 25 μM, 50 μM, 75 μM, 100 μM, 150 μM or 200 μM and allowed to incubate at 37° C. with constant shaking (300 RPM). Every 10 min for 30 min, 30 μl of sample was removed and Cathepsin S was deactivated by addition of 10 μl 2% trifluoroacetic acid. Cleavage products were separated after injecting 1 μl of sample into an Acquity UPLC (Waters, IL, USA) equipped with a C18 column, and running a linear gradient from 5%-95% Acetonitrile with 0.1% formic acid. Mass spectra were collected using an Acquity QDA mass detector, and representable spectra for analysis were generated from summing spectrum across a 3.6 s window of peaks identified on the total ion chromatogram (TIC). For characterizing cleavage kinetics to generate Michaelis Menten plots, the concentration of SIINFEKL in each sample was determined from a standard curve of UPLC-MS [M⁺H]⁺ for known solution concentrations. Cleavage rates were determined from the slopes of linear fits of the change in concentration of SIINFEKL over the 30 min cleavage time. Each cleavage reaction was set-up three times for independent replicates (n=3) and Michaelis Menten kinetics of the resulting data were fit using GraphPad Prism (GraphPad Software, CA, USA).

Oligonucleotide Synthesis and Purification. Oligonucleotides were synthesized utilizing standard phosphonamidite chemistry on a MerMade12 synthesizer with phosphate (PO) or phosphorothioate (PS) backbones, as indicated, at 5 μmol scales. Strands were cleaved from solid supports and deprotected using 3 ml of a 1:1 solution of ~40% ammonium hydroxide/~40% methylamine at 55° C. for 30 min. Strands were then purified using a C18 or C4 (for strands containing cholesterol modifications) column with reverse-phase HPLC, and peaks were collected as fractions. Fractions containing product were verified by matrix-assisted laser desorption ionization-time of flight (MALDI-ToF) using a Bruker rapileX Tissuetyper (Bruker, MA, USA). The dimethoxytrityl group of the purified product was removed by incubating product strands in 20% aqueous acetic acid for 1 hr at room temperature, followed by three washes with ethyl acetate. The final product was lyophilized and dissolved in diH₂O, and concentration was measured using UV-vis absorption at 260 nm, with extinction coefficients outlined in Table 1. Extinction coefficients to calculate DNA and DNA/peptide concentrations were generated by the OligoAnalyzer tool in IDT's Web site.

TABLE 1
DNA Sequences.

| | | | Molecular | Extinction | SEQ |
|---|---|---|---|---|---|
| | Back- | Sequence | Weight | Coefficient | ID |
| Strand | bone | (5′-3′) | (g/mol) | (M⁻¹cm⁻¹) | NO |
| CpG | PS | TCC ATG | 7824.8 | 181100 | 1 |
| 1826 | | ACG TTC | | | |
| | | CTG ACG | | | |
| | | TT | | | |
| | | (Sp18)₂ | | | |
| | | Chol | | | |
| CpG | PO | AAC GTC | 6763.6 | 206700 | 4 |
| 1826 | | AGG AAC | | | |
| Comp | | GTC ATG | | | |
| | | GA Sp(18) | | | |
| | | Thiol | | | |

Sp18 = spacer 18 phosphoramidite (Glen Research CPG; 10-1918).
Chol = cholesteryl-TEG anchor (Glen Research CPG; 20-2975).
Thiol = thiol-modifier 3 S-S CPG (Glen Research; 20-2933).

Oligonucleotide-Peptide Conjugate Synthesis and Purification. Thiol-functionalized oligonucleotides were reduced to generate a free thiol for future reactions by incubating with 100 mM dithiothreitol (DTT, Sigma) dissolved in phosphate buffered saline (PBS) pH 7.2 at room temperature for 1 hr. The solution was then concentrated and washed at least 3 times with diH₂O using a 3 kDa molecular weight cut-off (MWCO) Amicon spin filter to remove the DTT. To prepare peptide-linker conjugates, peptide with a free N-terminus on resin was washed 3 times each with dimethylformamide (DMF) and methanol (MeOH) before reacting with small molecule linkers, N-β-maleimidopropyl-oxysuccinimide ester (BMPS) (TCI America), or succinimidyl 2-(2-pyridyldithio)ethyl carbonate (SDEC, made using previous protocols¹) dissolved in DMF (Sigma) (5 equivalents with respect to the initial peptide loading on the solid support) with 2.5 equivalents of triethylamine (Sigma). GQI-M27 peptides were reacted with (2,5-Dioxopyrrolidin-1-yl) 2-azidoacetate (Broad Pharm). The solution was mixed at room temperature overnight followed by removal of the reaction solution and subsequent washing of resin with excess DMF and MeOH, three times each. Peptides were cleaved off the solid-support and deprotected for 1 hr at room temperature with either 94% trifluoroacetic acid (Sigma), 2.5% diH₂O, 2.5% ethanedithiol (Sigma), and 1% triisopropyl silane (Sigma) or 95% trifluoroacetic acid (Sigma), 2.5% diH₂O, and 2.5% triisopropyl silane for peptides with and without methionine respectively. The cleavage cocktail was removed using nitrogen, and the resin was suspended in DMF and filtered through 0.2 μm PTFE filter. The peptide product was precipitated by adding ~5-6 times diethyl ether and left at −20° C. for ~1 hr to precipitate. The solution was centrifuged and washed with fresh diethyl ether 3 times, dried, and dissolved in DMF. For the CSS-Adpgk I and M27 peptides which contained a free cysteine, the purified peptides were dissolved in DMF and activated using 2,2′-dithiodipyridine under gentle agitation for 30 min at room temperature, followed by precipitation by diethyl ether and drying. Peptide concentration was quantified using a commercially available colorimetric peptide quantification assay (Pierce, ThermoFisher) using standards generated from purified peptide stocks. Reduced DNA (750 nmol) was reacted at room temperature with the peptide-linker conjugates (7.5 μmol) in 70% DMF/30% diH₂O overnight. For peptide-DNA conjugates containing the hybrid linker-M27 peptides (GQI-M27), reduced DNA was first reacted with 10 equivalents of dibenzocyclooctyne-maleimide in 70% DMF/30% diH₂O overnight followed by extensive washing with diH₂O using 3 kDa MWCO Amicon spin filters to remove unreacted linker. Then the dibenzocyclooctyne-containing DNA was reacted with the azido-modified peptide overnight in 70% DMF/30% diH₂O. Following conjugation, solutions were diluted 5× with diH₂O and concentrated with 3 kDa MWCO spin filters followed by at least 4 washes with diH₂O. The final volume of product solution was brought up to ~500 μl, and mixed with 500 μl 8M urea (Sigma) in 2×TBE buffer containing loading dye (bromophenol blue) before being purified using preparatory scale denaturing (8 M urea) 15% PAGE gels. Gels were run for 30 min at 175 V, followed by ~3 hrs at 350 V. Gels were imaged under UV light atop a TLC plate, and bands corresponding to desired product cut out, crushed, and incubated with ~40 ml diH₂O overnight with constant inversion. The diH₂O was replaced every 8-18 hr two more times. Respective supernatants were combined and filtered though a 0.2 μm filter, concentrated with a 3 kDa MWCO spin filter and washed 4 times with diH₂O. Successful oligonucleotide-peptide conjugates were verified via MALDI-ToF.

Spherical Nucleic Acid (SNA) Synthesis. SNAs were synthesized following previously established protocols with slight modifications.¹⁻³ Briefly, 50 mg of 1,2-dioleoyl-sn-glycero-3-phosphocholine (DOPC, Avanti Polar Lipids) dried lipid films were hydrated with 3 ml 1×PBS. The solutions were subjected to 15 freeze-thaw cycles in liquid nitrogen and then sonication at room temperature. The liposomes were extruded using sequential high-pressure extrusion (Northern Lipids, CA) using polycarbonate filters with pore sizes of 200, 100, 80, and 50 nm; passing solutions through each pore size 3 times. The final liposome solution was concentrated using 100 kDa MWCO spin filters. Liposome concentration was determined using a phosphatidylcholine assay kit (Sigma), assuming that 50 nm liposomes contain 18,140 lipids.⁴ Liposomes were characterized by dynamic light scattering (DLS) and ζ-potential measurements using a Zetasizer Ultra (Malvern Panalytical, UK). Purified oligonucleotide-peptide conjugates and complementary 3′-cholesterol-terminated CpG DNA were mixed in a 1:1 molar ratio and lyophilized overnight. Samples were then dissolved in 1× duplex buffer (IDT) at 100 μM and slow-cooled to duplex the strands by heating the strands at 70° C. for 10 min (duplex Tₘ=56° C.), 23° C. for 1.5 hr, 4° C. for >1 hr. Duplex solutions were added to liposomes at a 75:1 ratio to maximize surface loading of oligonucleotides.² SNA synthesis was characterized by agarose gel electrophoresis, DLS, and ζ-potential measurements. Gel electrophoresis was performed using a 0.5% agarose gel stained with SYBR™ Gold Nucleic Acid Gel Stain (Invitrogen™, ThermoFisher) in 1×TBE buffer at 100V for 30 min at 0° C. Gels were imaged under UV excitation with a BioRad ChemiDoc MP imager (BioRad, IL, USA).

Cell Culture. All cells were maintained at 37° C. in a 5% CO₂ incubator. E.G7-OVA and bone marrow derived dendritic cells (BMDCs) were cultured with RPMI 1640 media (Gibco) supplemented with 10% heat-inactivated (HI)—FBS and 1% penicillin-streptomycin, referred herein as RPMI+/+. E.G7-OVA cells were cultured in RPMI+/+media additionally supplemented with 10 mM HEPES, 0.05 mM 2-mercaptoethanol, and 0.4 mg/mL G418. MC-38 cells were cultured in RPMI+/+media. B16-F10 cells were cultured in DMEM media (Gibco) supplemented with 10% heat-inactivated (HI)—FBS and 1% penicillin-streptomycin, referred herein as DMEM+/+.

BMDC Collection. Bone marrow cells were collected following a previously established protocol.⁵ Briefly, bone marrow cells were isolated from both femur and tibia of C57BL/6 mice by rinsing the bone marrow with 4-5 ml of RPMI+/+media. Following collection, red blood cells were lysed with 3 ml ACK lysis buffer (Gibco) for ~4 min, and cells were washed with DPBS and resuspended in RPMI+/+. Cells were plated on 10 cm² cell culture dishes in RPMI+/+supplemented with 40 ng/ml granulocyte-macrophage colony-stimulating factor (GM-CSF Biolegend) to differentiate DCs from the population.

BMDC Activation in vitro. BMDCs were collected from 10 cm² cell culture dishes, pelleted (1200 RPM, 5 min), and resuspended in RPMI+/+after which 2×10⁴ cells were transferred to individual flow tubes. Cells were allowed to recover for ~1 hr, following which they were treated with SNAs for 1 hr at 5 μM concentration by peptide and CpG DNA. After the 1 hr pulse, the cells were washed with media, pelleted (1200 RPM, 5 min), and supernatant was removed and replaced with fresh media. Cells were stored in an incubator for the specified amount of time, including the 1 hr treatment time. At each timepoint, cells were washed with DPBS, pelleted (1200 RPM, 5 min), and stained with fluorophore-conjugated antibodies for 15 min at 4° C. Staining included fixable blue live/dead (Thermofisher) and 0.5 μl each of BV711-CD11c (BD Biosciences, clone N418), PE-OVA-1H-2Kb (ThermoFisher, clone 25-D1.16), FITC-CD86 (BD Biosciences, clone GL1), and FITC-CD80 (BD Biosciences, clone 16-10a1) per sample. Cells were then washed with DPBS, pelleted (1200 RPM, 5 min), and fixed with 4% paraformaldehyde fixation buffer (BD Biosciences) for at least 15 min at 4° C. Flow analysis was performed after last timepoint utilizing a BD FACSymphony™ Cell Analyzer (BD Biosciences, CA, USA). Timepoints included 6 hr, 12 hr, 18 hr, 24 hr, 30 hr, 36 hr, 42 hr, and 48 hr. Flow data was gated on single and live cells, followed by CD11c+ cells. Gating for OVA-1H—2Kb+, CD80+, and CD86+ cells were based on untreated control groups. Single stain fluorophore controls were used to compensate for potential spectral overlap.

In vitro T Cell Stimulation. Spleens were harvested from OT-1 mice and strained though a 70 μm cell strainer with constant washing with DPBS. The single cell suspension was pelleted, and supernatant discarded. The pellet was resuspended in 3 mL ACK lysis buffer (Gibco) for approximately 4 min to lyse red blood cells after which, the cells were pelleted and supernatant discarded. Cells were resuspended at a concentration of 4×10⁷ cells/mL in DPBS and stained with eFluor 450 cell proliferation dye (ThermoFisher) for 20 min at 37° C. The volume was then doubled by addition of chilled RPMI+/+media, and the reaction was quenched by incubating the solution at 4° C. for 10 min. The cell suspension was pelleted, supernatant discarded, and cell resuspended at 1.6×10⁵ cells/mL in warm RPMI+/+media. 3×10⁵ cells were plated in round-bottom 96-well plates, and samples were immediately treated with SNA at concentrations ranging from 10 μM to 1 μM for 72 hrs. Following treatment, cells were washed with DPBS and stained with fixable blue live/dead stain and 0.5 μl of PE-CD8a (BD Biosciences, Clone 53-6.7) antibody for 15 min at 4° C. Cells were washed again with DPBS and flow analysis was performed utilizing a BD FACSymphony™ Cell Analyzer (BD Biosciences, CA, USA). Flow data was gated on single and live cells, followed by CD8a⁺ cells and proliferating cells based on negative controls. T cell proliferation dose-responsive curves were fit using a four-parameter Hill equation using GraphPad Prism (GraphPad Software, CA, USA). Dose responsive curves were repeated at least three times, with separate experiments representing responses from separate mice.

For cross-priming of T cells by SNA-treated BMDCs, BMDCs were collected from culture dishes 5-7 days after differentiation, and DCs were isolated using a magnetic biotin positive selection kit (Stemcell Technologies). A CD11c⁺ biotin-labelled antibody was used to select DCs and, after separation, purified DCs were counted using a Vi-Cell Blu cell viability analyzer. 4×10⁵ DCs were pulsed with 1 μM SNA by peptide and CpG DNA for 1 hr, following which cells were washed with DPBS and resuspended in RPMI+/+. Concurrently, splenocytes were isolated from an OT1 mouse. After dissociation of the spleen and lysis of the red blood cells with ACK lysis buffer (Gibco), the cells were counted and resuspended DPBS at 4×10⁷ cells/mL in DPBS and stained with eFluor 450 cell proliferation dye (ThermoFisher) for 20 min at 37° C. The volume was then doubled by addition of chilled RPMI+/+media, and the reaction was quenched by incubating the solution at 4° C. for 10 min. The cell suspension was pelleted, supernatant discarded, and cell resuspended at 1.6×10⁵ cell/mL in warm RPMI+/+media. 3×10⁵ cells were plated in round bottom 96-well plates, to which treated BMDCs were added at a ratio of DC:splenocytes of 1:50 and were allowed to culture for approximately 72 hrs, after which the cells were pelleted, washed with DPBS, and stained with fixable blue live/dead stain (ThermoFisher) and 0.5 μL of APC-Cy7-CD3 (BD Biosciences, Clone 17A2), FITC-CD8a (BD Biosciences, Clone 53-6.7), BV650-CD25 (BD Biosciences, Clone PC61), and BV711-KLRG1 (BD Biosciences, Clone 1F1) antibodies in approximately 100 μL for 15 min at 4° C. Flow analysis was performed utilizing a BD FACSymphony™ Cell Analyzer (BD Biosciences, CA, USA). Flow data was gated on single and live cells, followed by proliferating CD8a⁺ T cells. No-treat controls were used to gate for proliferating and KLRG1⁺ cells. Single stain fluorophore controls were used to compensate for potential spectral overlap using UltraComp eBeads™ Plus Compensation Beads (ThermoFisher).

Measurement of in vitro T Cell Stimulation. Spleens were harvested from OT-1 mice and strained though a 70 μm cell strainer with constant washing with DPBS. The single cell suspension was pelleted (1200 RPM, 5 min) and supernatant discarded. The pellet was resuspended in 3 ml ACK lysis buffer (Gibco) for 4 min to selectively lyse red blood cells. The cell suspension was pelleted (1200 RPM, 5 min) and supernatant discarded. The pellet was resuspended at a concentration of 4×10⁷ cells/ml in DPBS and stained with eFluor 450 cell proliferation dye (ThermoFisher) for 20 min at 37° C. The volume was then doubled by addition of chilled RPMI+/+media, and the reaction was quenched by incubating the solution at 4° C. for 10 min. The cell suspension was pelleted (1200 RPM, 5 min), supernatant discarded, and cells resuspended at 1.6×10⁵ cells in warm RPMI+/+media. 3×10\hat{ }5 cells/well were plated in round-bottom 96-well plates, and samples were immediately treated with SNA in PBS at concentrations ranging from 10 μM to 1 μM for 72 hrs. Following treatment, cells were washed with DPBS and stained with fixable blue live/dead stain (ThermoFisher) and 0.5 μl of PE-CD8a (ThermoFisher, Clone 53-6.7) antibody for 15 min at 4° C. The cells were washed again with DPBS and flow analysis was performed utilizing a BD FACSymphony™ Cell Analyzer (BD Biosciences, CA, USA). Flow data was gated on single and live cells, followed by CD8⁺ T cells. Histograms of eFluor 450 emission were used to calculate the percent of proliferating CD8⁺ T cells based on negative controls which encompassed <5% of signal. T cell proliferation dose-responsive data was fit using a four-parameter Hill equation using GraphPad Prism (GraphPad Software, CA, USA). Dose responsive curves were repeated at least three times, with separate experiments representing responses from separate mice.

In vivo Immunization to Measure 5-Week Build-Up in Immune Response. Female C57BL-6 mice were subcutaneously immunized with SNAs at 6 nmol treatments by CpG DNA and peptide according to the treatment schedule shown in each respective figure. Treatments included: BMPS-OVA₂₅₇₋₂₆₄-SNA, SDEC-OVA₂₅₇₋₂₆₄-SNA, GQP-OVA₂₅₇₋₂₆₄-SNA, and GQI-OVA₂₅₇₋₂₆₄-SNA. Volume of treatment injected was kept at 150 μl. Naïve mice were used as negative controls for gating of flow data. One week after the final immunization mice were killed, and spleens were collected for subsequent immune assessment.

Collection Procedure. Spleens were collected and temporarily held in RPMI+/+media until all spleens were collected, after which, they were passed through a 70 μm cell strainer which was constantly washed with DPBS. Cells were pelleted (1200 RPM, 5 min), after which the supernatant was removed, and the pellet was resuspended in 3 ml ACK lysing buffer for 4 min. The cell suspension was diluted with DPBS, and cells were pelleted (1200 RPM, 5 min). Cells were resuspended in 1 ml RPMI+/+, counted, and diluted to a concentration of 1×10⁸ cells/ml in RPMI+/+media.

INF-γ ELISpot Assay. Enzyme-linked immunospot assay for murine INF-γ detection was performed according to manufacturer's protocol (BD catalog no. 551083). Briefly, wells in the 96-well plate were coated with INF-γ capture antibody in PBS for ~16 hrs at 4° C. The solution was removed, and the plate was washed with 200 μl RPMI (+/+) and blocked with an additional 200 μl of RPMI for at least 2 hrs at RT. After, the media was removed and replaced with 2×10⁵ pooled splenocytes in 100 μl RPMI (+/+). An additional 100 μl of RPMI (+/+) containing either OVA₂₅₇₋₂₆₄ antigen (10 μg/ml), CD28 (BD Biosciences, clone 374.51) and CD3 (BD Biosciences, clone 145-2C11) antibodies (2 μg/ml each for a positive control), or media only (negative control) was immediately added, and samples were incubated for 48 hr at 37° C. in a 5% CO₂ incubator. Following incubation, the plate was washed, and the detection antibodies and substrate were added according to the manufacturer's protocol. The plate was dried overnight, and spots were counted on a CTL ImmunoSpot analyzer (ImmunoSpot, OH, USA).

Intracellular INF-γ Staining. Splenocytes (4×10⁶) from individually vaccinated mice were transferred to individual flow tubes and restimulated with 500 μl of RPMI (+/+) solution containing monensin (2 μM, Invitrogen, 00-4505-51), brefeldin A (5 μg/ml, Invitrogen, 00-4506-51), BV711-CD107a surface antibody (0.5 μl, BD Biosciences, clone 1D4B), and OVA₂₅₇₋₂₆₄ peptide antigen (10 μg/ml). Following incubation, cells were washed with 500 μl DPBS, pelleted (1200 RPM, 5 min) and stained for 15 min with fixable blue live/dead stain (ThermoFisher), and 0.5 μl each of APC-CD4 (BD Biosciences, clone GK1.5) and PE-CD8a (ThermoFisher, clone 53-6.7) at 4° C. Then, splenocytes were washed with 600 μl of DPBS, pelleted (1200 RPM, 5 min), and resuspended in 100 μl of fixation and permeabilization solution (BD Biosciences), and incubated for 20 min at 4° C. 300 μl of 1× permeabilization wash buffer (BD Biosciences) was then added to the cells, and the cells were pelleted (1200 RPM, 5 min) and resuspended in 100 μl of permeabilization wash buffer containing 0.5 μl of FITC-INF-γ (BD Biosciences, clone XMG1.2) and stained for 1 hr at 4° C. Cells were washed with 600 μl DPBS, pelleted (1200 RPM, 5 min) and resuspended in 100 μl DPBS and stored at 4° C. until analysis using flow cytometry. Flow data was gated on single cells, live cells, CD8+/CD4− cells, and INF-γ+/CD107a+ cells. Single stain fluorophore controls were used to compensate for potential spectral overlap using UltraComp eBeads™ Plus Compensation Beads (ThermoFisher).

T Cell Antigen Specificity and Memory Phenotyping. Splenocytes (3×10⁶) were washed with DPBS and stained for 15 min with fixable blue live/dead stain (ThermoFisher), and 0.5 μl each of APC-CD4 (BD Biosciences, clone GK1.5), PE-CD8a (ThermoFisher, clone 53-6.7), FITC-CD44 (BD Biosciences, clone IM7), and BV605-CD62L (BD Biosciences, clone MEL-14) at 4° C. Cells were washed with DPBS, pelleted (1200 RPM, 5 min), and fixed with Fixation Buffer (Biolegend) and stored at 4° C. before flow cytometry analysis utilizing a BD FACSymphony™ Cell Analyzer (BD Biosciences, CA, USA). Flow data was gated on single cells, live cells, CD8+/CD4− cells, and CD44+/CD62L+ cells. In further experiments, splenocytes (1×10⁶) cells were stained with fixable blue live/dead stain (ThermoFisher), and 0.5 μL each of APC-Cy7-CD3 (BD Biosciences, Clone 17A2), FITC-CD8a (BD Biosciences, Clone 53-6.7), BV711-KLRG1 (BD Biosciences, Clone 1F1), BV650-CD127 (BD Biosciences, Clone A7R34), R718-CD44 (BD Biosciences, Clone IM7), BV605-CD62L (BD Biosciences, Clone MEL-14), and OVA₂₅₇₋₂₆₄-specific pentamer (ProImmune) for 15 min at 4° C. Cells were fixed with fixation buffer (BD Biosciences) and stored at 4° C. before flow cytometry analysis utilizing a BD FACSymphony™ Cell Analyzer (BD Biosciences, CA, USA). Flow data was gated on single and live cells, followed by CD8a⁺ short-lived effector (KLRG1⁺/CD127⁻) and memory effector (KLRG1⁻/CD44⁺/CD62L−) antigen specific (Pentamer⁺) T cell phenotypes. Single stain fluorophore controls were used to compensate for potential spectral overlap using UltraComp eBeads™ Plus Compensation Beads (ThermoFisher).

Antigen Specific Cell Killing. Cytotoxic CD8+ T cells were isolated from harvested mouse splenocytes following the protocol for CD8a Positive Selection Kit (Stem Cell Technologies). Briefly, isolated cells were resuspended in media, counted, and added to a 96-well round bottom plate in effector (T cells) to target ratios (E/T) of 50:1, 25:1, and 12.5:1 with 5×10³ EG.7-OVA target cells that were pre-stained with efluor450 (ThermoFisher). Cells were co-incubated for approximately 15 hrs or 21 hrs at 37° C., and then collected and stained with 7-AAD (ThermoFisher) and FITC-Annexin V (ThermoFisher) for 15 min in the dark. Cells were immediately analyzed by flow cytometry utilizing a BD FACSymphony™ Cell Analyzer (BD Biosciences, CA, USA) to assess cells positive for eFluor 450, 7-AAD, and Annexin V⁺/7-AAD⁻ or Annexin V⁺/7-AAD⁺. Wells containing solely target cells were used as negative controls and for gating.

Tumor Bearing in vivo Efficacy Studies. Female C57BL/6 mice aged 6-8 weeks were inoculated with tumor cells by subcutaneous injection in their right flank with either 5×10⁵ E.G7-OVA, 5×10⁵ MC-38, or 1×10⁵ B16-F10 cells. Immunizations were administered at a dose of 6 nmol of each peptide antigen and CpG DNA by subcutaneous injection in the abdomen. Immunizations were administered as listed in the treatment schedule provided in respective figures. Blinded tumor measurements were made every 2-3 days starting on the first day of injection by measuring the length and width of tumors. Tumor volume was approximated with these measurements using the following equation: tumor volume=length×width²×0.5. Animals were euthanized when tumor volumes reached a width of 2,000 mm³ (E.G7-OVA) or 1,500 mm³ (MC-38 and B16-F10), or when the animal became moribund.

Software and Statistical Analysis. Matlab was used to screen peptides based off PICS analysis. TIC plots and summed mass spectrum were plotted using Igor Pro 8 (Wavemetrics, OR, USA). Curve fitting of Michaelis Menten kinetics, curve fitting of dose-responsive curves, and statistical analysis was performed using GraphPad Prism (GraphPad Software, CA, USA) and specific statistical analyses used are highlighted in the respective figure captions. For all cases, P values are depicted as follows: *P<0.05, **P<0.01, ***P<0.001, and ****P<0.0001. Allocation of animals to each group, administered immunization and measurements for studies were performed blind. Values in graphs are depicted as the mean±STDEV or SEM and this, as well as sample sizes, are indicated in the respective figure captions.

TABLE 2
Sequence Listing Table

| SEQ ID | | |
|---|---|---|
| NO | Description | Sequence |
| 1 | CpG 1826 | 5′-TCCATG ACGTTCCTGACGTT-3′ |
| 2 | CpG 1018 | 5′-TGACTGTGAACGTTCGAGATGA-3′ |
| 3 | CpG 2006 | 5′-TCGTCGTTTTGTCGTTTTGTCGTT-3′ |
| 4 | Complementary CpG 1826 | 5′-AACGTCAGGAACGTCATGGA-3′ |
| 5 | Complementary CpG 1018 | 5′-TCATCTCGAACGTTCACAGTCA-3′ |
| 6 | Complementary CpG 2006 | 5′-AACGACAAAACGACAAAACGACGA-3′ |
| 7 | OVA associated antigen | SIINFEKL |
| 8 | Human melanoma antigen gp100 | KVPRNQDWL |
| 9 | Murine H2Db | TAPDNLGYA |
| 10 | M27 melanoma neoantigen | LCPGNKYEM |
| 11 | CpG 2395 | 5′-tcgtcgttttcggcgcgcgccg-3′ |
| 12 | | GQPMESIINFEKL |
| 13 | | GQSMESIINFEKL |
| 14 | | GQIMESIINFEKL |
| 15 | Adpgk I neoantigen | ASMTNMELM |
| 16 | M27 neoantigen | LCPGNKYEM |
| 17 | GQI-Adpgk I | GQIMEASMTNMELM |
| 18 | GQI-M27 | GQIMELCPGNKYEM |

### REFERENCES

(1) Skakuj, K.; Teplensky, M. H.; Wang, S.; Dittmar, J. W.; Mirkin, C. A. Chemically Tuning the Antigen Release Kinetics from Spherical Nucleic Acids Maximizes Immune Stimulation. ACS Cent Sci 2021, 7 (11), 1838-1846. DOI: 10.1021/acscentsci.1c00779 From NLM PubMed-not-MEDLINE. Skakuj et al., J. Am. Chem. Soc. 2018, 140, 4, 1227-1230.
(2) Wang, S.; Qin, L.; Yamankurt, G.; Skakuj, K.; Huang, Z.; Chen, P. C.; Dominguez, D.; Lee, A.; Zhang, B.; Mirkin, C. A. Rational vaccinology with spherical nucleic acids. Proc Natl Acad Sci USA2019, 116 (21), 10473-10481. DOI: 10.1073/pnas.1902805116 From NLM Medline.
(3) Teplensky, M. H.; Evangelopoulos, M.; Dittmar, J. W.; Forsyth, C. M.; Sinegra, A. J.; Wang, S.; Mirkin, C. A. Multi-antigen spherical nucleic acid cancer vaccines. Nat Biomed Eng 2023, 7 (7), 911-927. DOI: 10.1038/s41551-022-01000-2 From NLM Medline.
(4) Banga, R. J.; Chernyak, N.; Narayan, S. P.; Nguyen, S. T.; Mirkin, C. A. Liposomal spherical nucleic acids. J Am Chem Soc 2014, 136 (28), 9866-9869. DOI: 10.1021/ja504845f From NLM Medline.
(5) Skakuj, K.; Wang, S.; Qin, L.; Lee, A.; Zhang, B.; Mirkin, C. A. Conjugation Chemistry-Dependent T-Cell Activation with Spherical Nucleic Acids. J Am Chem Soc 2018, 140 (4), 1227-1230. DOI: 10.1021/jacs.7b12579 From NLM Medline.
(6) Lutzner, N.; Kalbacher, H. Quantifying cathepsin S activity in antigen presenting cells using a novel specific substrate. J Biol Chem 2008, 283 (52), 36185-36194. DOI: 10.1074/jbc.M806500200 From NLM Medline.
(7) Biniossek, M. L.; Nagler, D. K.; Becker-Pauly, C.; Schilling, O. Proteomic identification of protease cleavage sites characterizes prime and non-prime specificity of cysteine cathepsins B, L, and S. J Proteome Res 2011, 10 (12), 5363-5373. DOI: 10.1021/pr200621z From NLM Medline.
(8) Schilling, O.; Huesgen, P. F.; Barre, O.; Auf dem Keller, U.; Overall, C. M. Characterization of the prime and non-prime active site specificities of proteases by proteome-derived peptide libraries and tandem mass spectrometry. Nat Protoc 2011, 6 (1), 111-120. DOI: 10.1038/nprot.2010.178 From NLM Medline.
(9) Dheilly, E.; Battistello, E.; Katanayeva, N.; Sungalee, S.; Michaux, J.; Duns, G.; Wehrle, S.; Sordet-Dessimoz, J.; Mina, M.; Racle, J.; et al. Cathepsin S Regulates Antigen Processing and T Cell Activity in Non-Hodgkin Lymphoma. Cancer Cell 2020, 37 (5), 674-689 e612. DOI: 10.1016/j.ccell.2020.03.016 From NLM Medline.
(10) Cuesta, S. A.; Mora, J. R.; Zambrano, C. H.; Torres, F. J.; Rincón, L. Comparative study of the nucleophilic attack step in the proteases catalytic activity: A theoretical study. Molecular Physics 2019, 118 (14). DOI: 10.1080/00268976.2019.1705412.
(11) Tzeng, A.; Kauke, M. J.; Zhu, E. F.; Moynihan, K. D.; Opel, C. F.; Yang, N. J.; Mehta, N.; Kelly, R. L.; Szeto, G. L.; Overwijk, W. W.; et al. Temporally Programmed CD8α+DC Activation Enhances Combination Cancer Immunotherapy. Cell Reports 2016, 17 (10), 2503-2511. DOI: 10.1016/j.celrep.2016.11.020.
(12) Ponte, J. F.; Sun, X.; Yoder, N. C.; Fishkin, N.; Laleau, R.; Coccia, J.; Lanieri, L.; Bogalhas, M.; Wang, L.; Wilhelm, S.; et al. Understanding How the Stability of the Thiol-Maleimide Linkage Impacts the Pharmacokinetics of Lysine-Linked Antibody-Maytansinoid Conjugates. Bioconjugate Chemistry 2016, 27 (7), 1588-1598. DOI: 10.1021/acs.bioconjchem.6b00117.
(13) Mahiddine, K.; Hassel, C.; Murat, C.; Girard, M.; Guerder, S. Tissue-Specific Factors Differentially Regulate the Expression of Antigen-Processing Enzymes During Dendritic Cell Ontogeny. Frontiers in Immunology 2020, 11. DOI: 10.3389/fimmu.2020.00453.
(14) Ten Brinke, A.; Marek-Trzonkowska, N.; Mansilla, M. J.; Turksma, A. W.; Piekarska, K.; Iwaszkiewicz-Grzes, D.; Passerini, L.; Locafaro, G.; Punet-Ortiz, J.; van Ham, S. M.; et al. Monitoring T-Cell Responses in Translational Studies: Optimization of Dye-Based Proliferation Assay for Evaluation of Antigen-Specific Responses. Frontiers in Immunology 2017, 8. DOI: 10.3389/fimmu.2017.01870.
(15) Teplensky, M. H.; Dittmar, J. W.; Qin, L.; Wang, S.; Evangelopoulos, M.; Zhang, B.; Mirkin, C. A. Spherical Nucleic Acid Vaccine Structure Markedly Influences Adaptive Immune Responses of Clinically Utilized Prostate Cancer Targets. Adv Healthc Mater 2021, 10 (22), e2101262. DOI: 10.1002/adhm.202101262 From NLM Medline.
(16) Moore, M. W.; Carbone, F. R.; Bevan, M. J. Introduction of soluble protein into the class I pathway of antigen processing and presentation. Cell 1988, 54 (6), 777-785. DOI: 10.1016/s0092-8674(88)91043-4 From NLM Medline.
(17) Embgenbroich, M.; Burgdorf, S. Current Concepts of Antigen Cross-Presentation. Front Immunol 2018, 9, 1643. DOI: 10.3389/fimmu.2018.01643 From NLM PubMed-not-MEDLINE.

1. A spherical nucleic acid (SNA) comprising:
a) a nanoparticle core;
b) a shell of oligonucleotides comprising one or more oligonucleotides attached to the nanoparticle core; and
c) a hybrid peptide attached to one or more oligonucleotides in the shell of oligonucleotides, wherein the hybrid peptide comprises a Cathepsin S-sensitive linker and an antigen.

2. The SNA of claim 1, wherein the Cathepsin S-sensitive linker is cleavable and traceless.

3. The SNA of claim 1, wherein the hybrid peptide is cleaved between a P1 and a P1′ position.

4. (canceled)

5. The SNA of claim 1, wherein the Cathepsin S-sensitive linker comprises a four amino acid sequence.

6. The SNA of claim 5, wherein the Cathepsin S-sensitive linker comprises Gln-X-Met-Glu, wherein X is an amino acid.

7. (canceled)

8. The SNA of claim 1, wherein the Cathepsin S-sensitive linker further comprises a Gly amino acid residue at the N-terminus of the Cathepsin S-sensitive linker.

9. (canceled)

10. (canceled)

11. (canceled)

12. The SNA of claim 1, wherein one or more oligonucleotides in the shell of oligonucleotides comprises a double-stranded oligonucleotide.

13. The SNA of claim 1, wherein the shell of oligonucleotides comprises DNA, RNA, or a combination thereof.

14. (canceled)

15. (canceled)

16. (canceled)

17. (canceled)

18. (canceled)

19. (canceled)

20. The SNA of claim 12, wherein one strand of the double-stranded oligonucleotide in the shell of oligonucleotides comprises the nucleotide sequence of SEQ ID NO: 1, SEQ ID NO: 2, and/or SEQ ID NO: 3.

21. The SNA of claim 12, wherein one strand of the double-stranded oligonucleotide in the shell of oligonucleotides comprises the nucleotide sequence of SEQ ID NO: 4, SEQ ID NO: 5, and/or SEQ ID NO: 6.

22. (canceled)

23. (canceled)

24. (canceled)

25. (canceled)

26. The SNA of claim 1, wherein the nanoparticle core is a liposomal core, a lipid nanoparticle core, a polymer core, a protein core, or an inorganic core.

27. (canceled)

28. (canceled)

29. (canceled)

30. The SNA of claim 1, wherein the antigen is a tumor-associated antigen, a tumor specific antigen, a neoantigen, or a combination thereof.

31. (canceled)

32. The SNA of claim 1, wherein the antigen is SIINFEKL, KVPRNQDWL, TAPDNLGYA, or LCPGNKYEM.

33. A hybrid peptide comprising a Cathepsin S-sensitive linker and an antigen, wherein the Cathepsin S-sensitive linker comprises Gln-X-Met-Glu, and wherein X is an amino acid.

34. The hybrid peptide of claim 33, wherein the Cathepsin S-sensitive linker is cleavable and traceless.

35. The hybrid peptide of claim 33, wherein the hybrid peptide is cleaved between a P1 and a P1′ position.

36. (canceled)

37. (canceled)

38. The hybrid peptide of claim 33, wherein the Cathepsin S-sensitive linker X is a hydrophobic amino acid.

39. (canceled)

40. (canceled)

41. (canceled)

42. (canceled)

43. (canceled)

44. A composition comprising a plurality of the SNAs of claim 1.

45. A composition comprising a plurality of the hybrid peptides of claim 33.

46. (canceled)

47. A pharmaceutical formulation comprising the SNA of claim 1, and a pharmaceutically acceptable carrier, diluent, stabilizer, preservative, or adjuvant.

48. An antigenic composition comprising the SNA of claim 1, wherein the antigenic composition is capable of generating an immune response in a mammalian subject.

49. (canceled)

50. A method of producing an immune response in a subject, comprising administering to the subject an effective amount of the antigenic composition of claim 48, thereby producing an immune response in the subject.

51. (canceled)

52. (canceled)

53. (canceled)


SEQUENCE LISTING (18 sequences)

SEQ ID NO: 1 - DNA, 20 bp, synthetic construct
  source 1..20: mol_type = other DNA
tccatgacgttcctgacgtt

SEQ ID NO: 2 - DNA, 22 bp, synthetic construct
  source 1..22: mol_type = other DNA
tgactgtgaacgttcgagatga

SEQ ID NO: 3 - DNA, 24 bp, synthetic construct
  source 1..24: mol_type = other DNA
tcgtcgttttgtcgttttgtcgtt

SEQ ID NO: 4 - DNA, 20 bp, synthetic construct
  source 1..20: mol_type = other DNA
aacgtcaggaacgtcatgga

SEQ ID NO: 5 - DNA, 22 bp, synthetic construct
  source 1..22: mol_type = other DNA
tcatctcgaacgttcacagtca

SEQ ID NO: 6 - DNA, 24 bp, synthetic construct
  source 1..24: mol_type = other DNA
aacgacaaaacgacaaaacgacga

SEQ ID NO: 7 - protein, 8 aa, synthetic construct
SIINFEKL

SEQ ID NO: 8 - protein, 9 aa, synthetic construct
KVPRNQDWL

SEQ ID NO: 9 - protein, 9 aa, synthetic construct
TAPDNLGYA

SEQ ID NO: 10 - protein, 9 aa, synthetic construct
LCPGNKYEM

SEQ ID NO: 11 - DNA, 22 bp, synthetic construct
  source 1..22: mol_type = other DNA
tcgtcgttttcggcgcgcgccg

SEQ ID NO: 12 - protein, 13 aa, synthetic construct
GQPMESIINFEKL

SEQ ID NO: 13 - protein, 13 aa, synthetic construct
GQSMESIINFEKL

SEQ ID NO: 14 - protein, 13 aa, synthetic construct
GQIMESIINFEKL

SEQ ID NO: 15 - protein, 9 aa, synthetic construct
ASMTNMELM

SEQ ID NO: 16 - protein, 9 aa, synthetic construct
LCPGNKYEM

SEQ ID NO: 17 - protein, 14 aa, synthetic construct
GQIMEASMTNMELM

SEQ ID NO: 18 - protein, 14 aa, synthetic construct
GQIMELCPGNKYEM